SEQUENCE LISTING (233 sequences)

SEQ ID NO: 1 - DNA, 2193 bp, Cannabis sativa
ttaaggtttttaagctgtttgagctttgttcttgtaatggcgttttgtgcattatcatttcctgctcatattagccgggcaactacaccagcaccttcagatcttcaaaaatctagttctttctcttctcggttttattggggagcagatctgctgaggccatctcaatacaaggtcaggaaaatacaaagtggggtttatgcatcactgtcagaaagtggagaatatcactcaaggagaccaccaactcctctcttggacaccataaattatccaattcatatgaaaaatctctctgttaaggagcttaaacaactatcagatgaactaaggtctgatgtcatcttcaacgtttctaacaccgggggtcacctgggctcaagccttggtgttgttgagcttactgtggctcttcattttgtcttcaatactcctcaggataggatactatgggatgttggtcatcagtcttaccctcataaaattctgactggaagaagagataagatgcacaccatgaggcagaccaacgggttagccggattcactaagcggtctgagagtgaatatgattgttttgggactggtcatagttctaccaccatctcagctggcttgggaatggctgttggaagggatcttaaaggaagaaagaataatgttgtggctgtcataggtgatggtgccatgacagcaggtcaagcttatgaagccatgaataatgccgggtaccttgattccgacatgattattattcttaacgacaataaacaggtttctttacctactgcctctcttgatgggcccataccacctgttggagctttgagtagtgctctcagtaggctgcaatcaaacaggcctcttagagaactaagagaagtagccaagggagttactaaacaaataggtggatcagttcatgaattggctgcaaaagttgatgaatatgctcgtggaatgataagtggttctggctcaacattgtttgaggagcttggactctattatattggtccagttgatggtcacaatatagatgatcttgtttccatactagaggaggttaagagcactaaaacaacaggtccagtcttgatccattgcatcactgagaaaggaagaggatatccatatgcagagaaagctgctgataagtatcatggggtggccaagtttgatccagcaactggaaagcaattcaaaggcacttctaacacacagtcatacactacatactttgctgaggctttggttgcagaagcagaggcagacaaagatgttgtggccatccatgctgcaatgggtggtggaacaggcttgaatctcttccttcgccgtttcccaacaagatgttttgatgttgggatagcagaacagcatgctgttactttcgctgctggtttggcttgcgagggccttaagccgttttgtgcaatttactcatctttcatgcagcgagcctatgatcaggtagtacatgatgttgatttgcagaagttgccagtgagatttgcaatggacagagctggacttgttggggccgacggccctacacattgtggtgcttttgatgtaactttcatggcatgcctcccaaacatggttgtgatggctccttccgatgaggcagagctcttccacatggttgccaccgctgctgccatagatgacagaccaagttgtttccgttaccccagaggaaatggaattggtgttccattacctcaagggaataaaggaactcctcttgagatcggaaaaggcagggtattggttgaaggggagagagtagcacttctaggctatggaacagcagttcagagttgtttggctgctgcagccttagtagaacctcacggtctacggctaacagttgctgatgcacgattttgcaagcctttggatcatgccctcattcgcgaactagcgaaaaatcacgaggttttgattacagtggaagaaggatctataggaggttttggatctcatgttgctcagtttatggcccttgatggccttcttgatggaaaaacaaagtggagaccaattgttcttcctgaccgatacatcgaccacggttcgcctgctgatcaatatgtcgacgcgggtctcacgccacctcacattgcagccacagttttcaatgtactaggacaaacaagagaggccttgaaggttatgacaacatga

SEQ ID NO: 2 - DNA, 1818 bp, Cannabis sativa
aattacccagttcacatgaagaatttatccacacaggatcttgaacagctagcagcagagcttagagcagatgtagtccatacagtatcaaaaacaggtggtcatctgagtgcaagcttgggagttgtggagctcactgtagcactgcatcatgttttcaatacccctgatgataaaatcatatgggatgttggacatcagacatacccgcataagattcttacaggaaggaggtctcaaatgcataccattagaaagacttctggtctagcagggtttcccaaaagagatgagagtgtttacgatgctttcggtgcaggtcacagttctacaagcatatcagcaggccttggcatggcagttgccagggatcttctgggaaagaagaacagtgttgtttctgtgattggagatggggccatgactgcaggaatggcatatgaagccatgaataatgccggctacttggacgccaacttgattgttgtattaaacgacaataaacaagtttctttaccaactgctactcttgatggtcctgcaaccccagtgggagctctaagtggtgctttgactaagcttcaagcaagcaccaagttcagaaaacttcgcgaagctgcgaaaaccatcacaaaacaaattggagggccagcacatgaagttgcagctaaagtagatgagtatgctagaggaatgataagtgcttctgggtcaacactctttgaggagcttgggttgtattatattggtccggtggatggacataatgttggagatttagtcaccatttttgagaaagtgaaatcaatgccagcgccaggaccagtcttgatccacatcgtcacagagaaagggaaaggctatcccccagctgaagtagcacctgataaaatgcatggagttgtaaagtttgacccaacaacaggaaagcaatttaagtccaaatcgtcgacactttcatatactcaatactttgctgaatctctaataaaagaagctgaagaagatgacaagattgttgccatacacgcagcaatgggtggtggcactggtctcaattatttccagaagaaatttcctgatcgttgctttgatgtggggattgctgagcaacatgctgtcacgtttgcagctggattagctgcagagggtctcaaaccattctgtgccatatactcatcattcctgcaacgaggatatgatcaggttgcacatgatgtagaccttcaaaaattacctgtccgttttgcattggatagagctggcatggttggcgcagatgggcctacccactgcggtgcatttgatatcacctacatggcctgcttgcccaacatggttgtcatggctccatcagatgaggctgaacttatgcacatggtggccacagcaacagctatagatgacagacccagttgcttcaggtttccaaggggcaatggaattggagcaaagcttccagctaataataaaggaactatacttgagattggaaaaggcagaatattaatggaaggcagcagagtagctattttgggttatggttctattgttcagcaatgtgtggaagctgcaagcatattaaagaaacaagacatttcagtgacagtagctgatgcaagattttgcaaaccattggatacaaatctcataagacggttagccaacgagcatgaaatcctaatcactgccgaagaaggttctattggaggctttgggtctcatgtgtcacactttctaagcttaagtggacttcttgatgggtctttaaagttgagagcaatggttcttcctgatagatacattgaccatggatca

SEQ ID NO: 3 - DNA, 1419 bp, Cannabis sativa
gctctgaacttgttatccccagctgaagtgaaggctctatcctttttggactccaccaagtccacccgcttccctaagctgtgtccaggtggaattactttgcatagaaaggattgcagagtaccacttagaagaagagttcattgttcgttgcagcaacctcctccagcttggccaggaagagctattccagagcaagatctttgtaattggaatgtcccaaagcctatatctattattggctctactggctctataggaactcagacactggacattgtggcagagaatccagataaattcagaatagtgggacttgcagctggttcgaatgtgacacttcttgcagaccaggtgaagagattcaagcctcaaatagttgctattagaaatgaatcattaattggtgaactgaaagaggccttagctgatgtggaagaaatgcccgaaattattcctggggaacaaggagtaattgaggttgcccggcacccagatgcagtcacagtggttacaggaatagtaggttgtgctggattacagcctacagttgctgcaattgaggcaggtaaacacatagctttagccaataaagagaccctgattgctggaggtccattcatccttcctctagctcacaagcataacataaaaattcttcctgccgattcagaacattcggcaatattccagtgtatccagggcttgcctgatggtgcactacggcgtatcattttgacagcatctgggggagctttcagagattggccggttgaaaagctaaaagatgttaaggttgctgatgctctgaaacatccaaactggagtatgggaaagaaaatcactgttgattctgctacccttttcaataagggtttagaagtcattgaagcccattatctatttggagcagactatgacaatattgacattgtaattcatccacaatctataatacattctatgattgaaacacaggattcttctgttctggctcagttggggtggcctgacatgcgaattccgattctctataccatgtcatggccagacagaatatactgttctgaagtaacttggcctcgacttgatctttgcaagcttggttcgctgacttttaagagtcctgacaacaagaagtacccatccatgaatcttgcctattctgccggacgtgctgggggcaccatgaccggagttcttagtgcagccaatgagaaagctgtagagatgtttattgatgagaagataagttatcttgaaatcttcaaagttgttgagctaacatgcgacaagcatcgatcagagatggtgacttcaccttctcttgatgaaattatccactatgactcgtgggcacgagagtatgcaactactagtttgaagagttcttccagtccaagacctgttacagcatga

SEQ ID NO: 4 - DNA, 978 bp, Cannabis sativa
tttattataacacttttctccatttccgaaaagaaaaaagtcagtagagaaaaacgcaacatggcgttacttgcaatggaccttactttctcttctgcttctctttcttcttcttcctacaatgctgctcctctactatttccttctattcgcccatcctctcaatccattgttcgatgcccagtccatgaggtgggattcagggggaaatgcagaatttccaagataaggttcgctcgctgctctgcaaatgttggccaaaagcctggtgttgtggaaaagaaaagcgtttcggtggttcttctggcaggtgggaagggtaaacggatgggggccaacatgccaaagcagtatcttccacttttagggcaaccaattgcactgtatagcttctacactttttctaaaatgattgaagtgactgaaattgttgtagtttgtgatccctcttacgaggatatctttgaagattccaaagacaagatccatgttggacttaaattcgctctgcctggaaaggaaagacaggattcagtttatagtggacttcaggcaattgatccaaactctaagcttgtgtgcattcacgattctgctagacctttggtaacaacagaagaagttaaaaaggtcattgaagatggttggttgcatggagcagctgtacttggtgttcctgtcaaagctacaatcaaagaggcaaacaatgcatcttttgtaactaaaacgttggacaggaaaaaactttgggaaatgcagacaccccaggtgatcaaacccgagttgctcaaggaaggatttgagcttgtaaatagggaaaatctggaagtgactgatgatgtgtctatagtggaacaccttggacatcctgtatatataacggaaggtgcttacaccaacatcaaggttactactccagatgatttattgcttgcggagagaatattgagtatgaactctgtaaaggctgttgcataa

SEQ ID NO: 5 - DNA, 1227 bp, Cannabis sativa
ttagttcatacttacctctccacaactaaaatggcttcctctcatattctctgccacaacaacgtttttaatctttctcccaatccttttaggaacaggggtctctcttccttaaactcaaatgggttttgtttttttggttcgaaatctagaatttcgaggccttcatctctcaaaattgtggtttctgaaagaagacaagttgagatagtttatgatgctgatgaaaggataaacaaattggctgatgtagtggacaaggaagcgcctctttctaggctcactcttttctcaccttgcaagattaatgttttcttgagaataactagcaaaagggaagatgggtatcatgatttggcatccctctttcacgtgataagtcttggagatgtgcttaagttctctttgtctccttcaacaaagaaagattctttgtcaacgaatgcctctggggtaccacttgatgatagaaatttgattatcaaggcccttaatctttaccgaaagaaaactggtacaaacaaatacttttggattcatcttgacaagaaagtgcccactggagcagggctaggtggtgggagcagcaatgctgcaacagcactatgggcagcaaatcagttcaatgattgtcttgttactgaaaaggaattgcaagaatggtcaagtgagattggttcagatgttcctttctttttctcccaaggggcagcctattgtacaggtcgaggtgaggttgttcaggatattctaccacctgtaccattaaacattcccatggttctcataaagcccccagaagcatgttcaacagccgaagtttataagcgttttcggttggataaaaccagtaatagcgatcctttacaattgctccacaagatctcaagtgatggaataagtcaagatgtctgcatcaatgacttagaacttcctgcctttgaagttcttccatctcttaagagattgaaacagcgtataattgcagctagtcgtggacaatatgatgctgtttttatgtctgggagtggaagcaccattgtcggggtcggttccccagatccacctcagtttatatatgatgatgaggactacaaggatgtgtttttgtcagaggccaactttctgactcgagaagcaaatgaatggtacaaagaacctgcttcagctagcgcttgtagcccttcagatgatttctctcgtaatttttcctcctctgtcgagtaa

SEQ ID NO: 6 - DNA, 1686 bp, Cannabis sativa
gagtcagcactgaaccgagaccacatacccgcgacctcgagcttcgacggccatggcggcggcgacacccgtgtgcgcttctactattccagcacagtactccagtaactccgtcaaatcattccatcactcccatttcacggttgcagttcccagaaatctcttctcatcgtcctcatcatctcatgtgtctttgagacaatccaaaacgacgccgctttcggctctgccttctgtgtcagccgccggaaccgccgccttggaagcggagcaaactccgtctgaggtatccacgactccctcaaagattcttccttttcgggtcggtcacgggttcgaccttcatcgattggagcccgggtatcctttgataattggaggcattaatatacctcacgagaaaggatgcgaggctcattctgatggtaatctctcgcccctgaaatgggtttatttcagtttctggattaagttgctaaaaactttcaatttttgttgtttggttgctgagaaaaaaagcatttgtgtactttttcaatgcagtttcttggggtttctttctaaagagaaaattatagatatattctttgatttgtgcttgcaggggacgttttgcttcattgtgtagttgatgctattttgggtgctttggggcttccggatattggacaaattttcccggattctgatcccaaatggaaaggggctgcatcatcagttttcatcaaagaagctgtaagttgttattgtttggttaagttatgcaactttgagccaattttgtttgaattttctatgcattttactctttattgaattgtcttgttctttcaaaccttcattcatatacagatttaacttgaataagttcttggtatatttctttttctctgttcaaacataaattctaggcaagagatgatttttgtttttttacataatgttcactaagaatttgagactaagttttgccattttcaacacaaacccaaatgaaagagactattggttaatagcctttgataaagtcttatcgatctctctcgaggtctgaccttttatttgaagacaataatgaataaacgtagaatttatacactactcattatgctgaaattatcctttacgatatacttgaagttttttgtttgaaatattgttgcgtgtccaaaagggaacaatgtctctttgtgacaaacaatggaacacaattatagaaaatcaaagtcactctgaactggttagtagttgatacattataagcatattaatatcttatccgttttaggtgagactgatggatgaggcaggttacgagcttggaaatttagatgcaacattaattcttcaaagaccgaagctaagtccacacaaggaggctatcagagccaacttatctgaacttctaggagctgaccctgcagttgttaatctgaaagcgaaaactcatgaaaaggttgacagcctcggagaaaatcgaagcattgctgctcacactgtggttcttcttatgaaaaagtagactgatgaagaagaagaagaagaagaagaagaagaagaaggcttggaaatcctggtcaaaaacatgtggttagtagttgcaactttcatactaatacaattcttacagaaactattacattttctatgaatcaggttatatgtaaaggtcaccttacatgtgtc

SEQ ID NO: 7 - DNA, 2247 bp, Cannabis sativa
gcttttgagtttgtggaaatggcttctggagctgtaccagcatcaatttcatgtctgaaaagcagagactctggcttgagctttgctaaaagttctgattttgtgaggctttctgatttaaagagggttggttcatctagaacaagagtttcagttatccgaaattcgaatcctggttcagatattgctgaacttcagcctgcatcaaaaggaagccctctattagttcctagacagaaatactgtgagtccttacacaaaactgttaggaggaaaacgcgaactgtgatggtgggaaatgtggctcttggcagtgagcatcccataagaattcaaacgatgacgacaaatgataccaaggatgttgctggaacagttgaagaggtgatgagaatagctgataagggagctgatattgttcggataacagttcagggaagaaaagaagcagatgcttgtttcgaaataaaaaattcacttgtgcagaagaattataatatccctcttgtggcagatattcattttgctcccccagttgcattaagagttgctgaatgcttcgataaaattcgtgtcaatcctggaaatttcgctgacagacgggctcagtttgagacgcttgagtacacagacgacgactatcagaaagaacttgagcatattgagcaggttttttctccattggttgagaaatgtaagaaatatggtagagcaatgcgtatcgggacaaaccatgggagtctttcagatcgtatcatgagctactatggagattctcctaggggaatggttgaatctgcatttgagtttgcaaggatttgccggaagttggatttccataattttgtgttttcgatgaaagcaagcaacccagttgtcatggttcaggcgtatcgtctacttgttgctgaaatgtatgtccagggctgggactatccacttcacttgggagttactgaagcgggagaaggtgaggatggacgaatgaaatctgcaattggcatcgggacccttcttcaggatggtttgggtgatactatcagggtttcactcactgaaccgcccgaggaggagattgatccctgcagaaggttggccaatttgggtacaaaagcagctgatcttcagcaaggagtggctccatttgaagagaagcacaggcattattttgattttcaacgacgaactggtcaactgcctctacagaaggagggcgatgaggttgactatagaggtgctctgcaccgtgatggttcggttctcatgtcagtgtctctcaataacttaaagatgcccgagctactatacaggtctctagcagcaaagcttgtcgtcgggatgccatttaaggatctggcaacggtagactccatcttattgagacaacttccacctattgacgatgacaacgctcgattagctctcaaaagattgatagacataagtatgggggtcataactcctttgtcggagcagctaacaaagccattgccaaatgctatggttttggtaaatcttaaggagttatcatctggtgcacacaagcttttgccagaaggcacgcgtttggttgtatccttgcgcggtgatgaaccttacgaagaactcgagattctcaaaggcgttgatgatgttgttatgattcttcatgatcttccgttcgatgaacataaaattagcagagtccactcagcaagaaggttatttgagtatctatcagataattctcttaactttcctgtaatacaccacattcaatttccaaatggaatccacagggatgacttagtcatcggtgcaggtagcaacgctggtgcccttttagtagatggactcggggacggtatcctcttagaagccccagatcaggatttcgattttcttagaaatacttctttcaacctacttcaaggttgtagaatgcgaaatacaaagacggagtatgtctcgtgcccatcctgcggtagaactttgtttgaccttcaagaaattagtgcagagattcgagagaagacatcacacctgcccggtgtctcaattgcaatcatgggttgcattgttaatggacccggagagatggctgatgcagacttcggttatgtcggtggtgctcccggaaagattgacctttacgttggaaagacggtagtgcagcgtggaatcgcaatggaacaagcaaccgatgcattgattcagctaataaaagatcatggccgatgggttgaaccaccctcagacgaagaatga

SEQ ID NO: 8 - DNA, 1224 bp, Cannabis sativa
ttcgatgcgaaagtgttccgtaagaacttggtccgaagcaagaactacaatcggaaaggttttggccataaggaagagacccttcaactcatggacagcgagtacaccagtgatattataaagactttgaaggataatggaaatgagtacaggtgggggaacgtgacggtaaaattggccgaagcatatgggttttgctggggtgtggagcgagctgtccaaattgcttacgaagcaaggaaacagttccccgaagaaaagatttggattacaaacgaaattattcataatccgacagtcaacaagagactagaggaaatgaaagtggaaaatattccaattgatgaagggaggaaacaatttgagattgtaaacaagggtgatgttgtgatattgcctgcttttggtgctggagtggatgagatgttggctttgagtgataggaatgttcaaattgttgataccacatgcccatgggtttccaaggtttggaatacagtcgagaaacataagaaaggtgaatacacttccattattcatggtaaatatgctcatgaggagactatagctactgcatcttttgctggaacttacattattgtaaagaacatgaaagaggcaatgtatgtctgtgattatattcttggcggtcaacttgatggatccagctcaacaagagaggagtttatggagaaatttaagaatgcagtttctaagggatttgatcctgacaaacatcttgtgaaggctggtattgcaaatcagactacaatgctcaagggggaaaccgaagagattgggaaactggttgagaggactatgatgcaaaagtacggagttgaaaacattaatgaacacttccaaagctttaacacaatttgcgatgcaacccaagagcgtcaagatgcaatgtacaagatggtggaggaacgtattgaccttatgttagttgttggaggatggaactctagtaacacttctcatctacaagagattgcagaggaacgaggtattccctcgtattggattgacagtgaacagagaataggtcctggaaacaagatagcctacaagctaaatcatggagagttggttgagaaagagaactggttaccagagggtcccatcacggtcggtgtaacatcaggtgcttctactccagataaggttgtggaagatgttctcatcaaggtgtttgaccttaagagcgaagaagctttgcaa

SEQ ID NO: 9 - DNA, 915 bp, Cannabis sativa
tatataattataatatactcgattcaacaatggcggagatcacatgcgacaatgaccctccttctaaataccactgccaagctctacattgcccccagaactcttccctttacctcttcctctacctttgctcgtagcccttttctccgaatcccctcccttcttaagcccttgtctcctctcaccgctagggtctctctttcttccaccatgggagactctgccgacgctggaatggacgctgtccagagacgccttatgtttgatgatgaatgcattctagtggatgagaatgaccgagttgttggtcatgatacaaaatataactgtcacttgatggaaaagattgaaaaggataatttgctacacagggctttcagtgtgttcttgttcaactcaaaatatgagttgcttcttcagcaacgttctgcaacaaaggtaacattccctcttgtgtggacaaacacctgttgtagccacccgctctaccgtgaatctgagcttatcgatgaggagtcccttggagcaaggaatgcagcacagagaaagcttttagatgagctgggtattcctgctgaagatgtgccagttgatcaatttaccccactaggcaggatgctgtacaaagctccttctgatggcaaatggggcgagcatgaacttgattacctgctcttcatcgtccgggatgttagtgtcaatccaaatccagatgaagtagctgatatcaagtatgtaaaccgggacgagttgaaagagttgttgaggaaagcagatgctggggaaggaggcttgaagctatccccttggttcagactggttgtggataatttcttgttcaagtggtgggaccatgttgagaaaggcacacttaaggaagttgctgatatgaaaaccattcacaagttgacttaa

SEQ ID NO: 10 - DNA, 1182 bp, Cannabis sativa
cacagagtgtctttgctttgttcattttcacaaaatcaaaaagcttcaatctttgtcaagactaagaagatgagcactgtaaatctcacatgggttcaaacctgttccatgttcaaccaaggaggtagatccagatccttatcaactttcaatctcaatctctaccaccctttgaaaaaaacacccttttcaatccaaaccccaaaacaaaaacgacccacttcaccattttcatcaatctcagctgttctaaccgagcaagaagccgttaaagaaggcgatgaagaaaaatccatcttcaatttcaagtcttacatggtccaaaaagccaactcagtcaaccaagctttagactcagccgttttgctcagagatcccattatgatacacgagtccatgcgttactcactcctcgccggaggaaaacgagtcagacccatgctctgtctctcagcctgtgaactcgtcggcggaaaagaatccgtagccatgccggctgcctgcgccgtcgaaatgatccacaccatgtctctaatccacgacgacctcccttgtatggacaacgatgacctccgccgtggaaagcccacaaaccacaaagtcttcggagaagacgtggccgttttagccggcgatgcacttttagcctttgcttttgagcacatggcggtctctaccgttggtgttccggcagccaagattgtcagggcgattggtgagcttgctaagtcaattgggtcagaaggattagtggctggtcaagtggttgatattgattcagagggtttggctaatgttgggcttgaacaacttgagttcattcatctccataagactggggctcttctagaagcttctgttgttttgggggctattcttggtggtggtacagatgaagaagttgaaaaacttaggagctttgctaggtgtattggcttgctttttcaggttgttgatgacattcttgatgtgactaaatcttctcaagaattgggtaaaactgctgggaaagatttggtggctgataaggttacttatccaaggctaatgggtattgacaaatcaagagaatttgctgagcaattgaacacagaagccaaacagcatctttctggttttgatcccataaaggctgctcctttaattgctttggctaattatattgcttataggcaaaattga

SEQ ID NO: 11 - DNA, 981 bp, Cannabis sativa
acgcagaggcctagttacgggggagcttcatcaatggcggtttataatctatcaattaattgcagtccaagatttgttcatcatgtttacgttccacatttcacatgtaaatccaataagtcgttaagtcacgtacccatgagaataaccatgtccaaacagcatcatcattcttattttgcctccacaacagccgatgtagatgcccatctcaagcaatccatcactatcaagccaccactctcagttcacgaggccatgtacaatttcatcttttccacacctccgaatttagcaccgtcattgtgcgtggcggcgtgtgagcttgtcgggggccaccaggaccaggccatggcagcagcctccgccttgcgcgtcatccacgcagccatcttcactcatgaccacctccctttaacgggcaggcccaatccaacaagtcctgaggcagcgacccacaattcttacaacccaaatattcagctccttctcccggacgcaattgtaccttttgggttcgaattgttggccaattctgatgaccttacccataataaatcagatcggattttgcgggtcattgtagagttcacacgcacctttggatcacgaggaactattgatgctcaataccatgagaagctagccagtagatttgacgttgatagtcatgaagccaaaactgtcgggtggggccattatccctctttgaagaaggaaggtgcgatgcatgcatgcgctgctgcatgtggggccattcttggagaggcacatgaagaagaggttgagaagttgagaacttttggtctttatgtgggcatgattcaaggatatgccaatagatttataatgagcagcacagaagaaaagaaagaagcagatagaatcatcgaggagttaaccaatttggctcgccaggaactaaaatatttcgatgggagaaacttagagccattttcaacctttctttttcgtctatag

SEQ ID NO: 12 - DNA, 1026 bp, Cannabis sativa
tattggagtatcatcaaaagagtagtactctcccaatatgggtttgatagctctggtggcttcatttgcatggtaatgaggtattgttgcgaataaatggtgtgccatatgagcatctgtaaggtgatggaaaaccctattgagaattgatccatagtttcgatcaattgttgacaatgctcctttcaaccaatcccactctgacgagtcataatgaggcaatgaagagtgagtatgattcaagtatgcgattgtcacaaggatgctattccctgtaaacaatggcaccccatacatgaacacaagccaactcaaccctttagtcgagccaagctggtataacaccaatgtggtgatgaaaacaccgatatctgaaatcaatatatgaagcctttcactttttgagtagagtggagagtaaggatcataatgacatgcagaacgatcatattgtttgcctgtaagattgaaacctaagtacaaaggagaaccaaggaacactataatagaaagctttagggctcttcctagtggattgtccaagtaatgtttgtagagccatgcgatttgagattcggggactggaacaaacaattgatcatgttcaagggagccgatgtttgaatgatggcgacgatgactatacttaaaagagaagtatgggaagagaagagcggagtggaggacaaagccaacagtgtcatccacccattggtggtcactgaaagcttggtgaccacaatcatgaccaaggacccaaataccagcaaaaatgctgccttggaagatccaataaagtggccaagctaagtattggagcgggtgtgggagaagatggaagtaagaggttgctatgtagtagaacaaaaagacgaaaaaaaggtcttgaaggacatgagagaaggaacgaagaagagaacggttgaagcaatggggtggaatggctttcttgagttggcttagtgtgaatggtggtgtggtgtgtggtgctcgctcaagtctactatttttgccaccggctcccat

SEQ ID NO: 13 - DNA, 3999 bp, Cannabis sativa
  misc_feature (778)..(794): n is a, c, g, or t
  misc_feature (1092)..(1802): n is a, c, g, or t
  misc_feature (3135)..(3702): n is a, c, g, or t
atgttgaagcctcctcatcaagtagttcaaaatttgaaatatgagaaaaccctagttcttttgaacaagccattcatccatggctacaacggggctattatcggtatcaactctcggctatttccagtaaaacctaaaaccaaaagacgagtcgcttcatcatcatcatcatcatctcccggaaccaaaaatattattaaagcttctttattttctccaatggagaagaagaatacagctagggtttcggttagtgtggcggtacaacgtgtgactccaaagttttggagatttgaattgtctgagaaaatccaagatggacttgataggcttgatgatcttctagggctaaactctttaagtattgagctagttagtgctcaaaaagatccaggtaagtttgttgagctagaattacttttattcgtatatatatcgatcttattagggatgttttatatttttggtacctagcttagttacgttgaatattgtagttataccagacatttgttaatttcgaaactttaaaatgtttatattgtcaaatttaaaattcaaaatagttaatcaaaacacgtataaaaatttactaaattacgcattatttaaatattattttcagcactgtaaattatttaaaattttcatatcaactataataattatatacgttgttacatataattaaaattaagttgtaaggtaggaaacacccctacaataaaagtgaaagtgatgctgcaatgtttctatgtatataataatcttttttttnnnnnnnnnnnnnnnnntaattttagcatattaaatgatgtgttaattatgattgatgatgatgatatacatcttaattatgaactttaattagaaatatgaagataatatatacatgaaattgacttgtgaatttataactgacacgcattttaatttttttaaaaaataaatagggacagaaaaaaaaaatgaaattgacaggacctgaaactagtgaatttgattcggataaggaagcaaatagtacttaatttaaagattgtggttggaattaatataggaaatttaaggaaatcaaaataaaattaaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgcgacggggaaagaacgaacggttaaaggttttccaaaaagacccgactttaacatattttcatcagatgatgtaaaatacgaagcaaaatttgacataccgaaagattttggagaagtgggtgctataatcgtcgaaaatgatttcgaaagagaaatatctttaaagaatattttactcgaagacttgccctccgaaccaagcacccttgaattctcttgcaactcgtgggttcagtccaaacatgatgttcctactgatcaacacaagagagtcttcttctctaataaggtatgtagtgattcatttcaatctctacaatgactatgaataatgtcatcagtatagtccaaaaaattgtcaataaaagttgtactgacaattaatcatattaaatttatataataattcacactacaacaaaaactgtcatcaggagcaatttatgtagtaattcatattccagaagattatttggcgtgactaatcattcttgatcgtgcctgaaaatcgatatcaataacaaaatataacaacgtgccaaataatatcctcagtacaatctagaaaattatcaataaaaactgtactgacaattaatcataggaatgagttttggtatgactttgtccatagaagttaacctctacggacaaagcgatattctcattcctacaaggagagtatgtcatccttgtagatttagatgtaacgtccccgcttcaagcctccattgggtccttacacccacggaatgaatggctcttatacacgagtacgtcactctggctgcttcatggactgatgactaaccctaccgaccaacacgagtgttttcagcgtgctttgtcctcacgcatcctaggaaaacttcccaggaggtcacccatccttgaaattgctccaggccaagcacgcttaactatggagttctttcgagatgggctaccgaaaaataagatgcaccttgttgacatagatagtaccaatcaatccatttaagccctcttcaactgtgtagtcccatacctacacagtctcataatcatcccacttgaccaattccccaggcggtgtggaattgcacagcttacccggtgtttccccttacggatcacgggactactgactgtcacaatcacccccccttacagggtccgacgtcctcgtcgaccacacttccggctgggtcaaggctctgataccatttgtaacgtccccgcttcaagcctccattgggcccttatacccacggaatgaatggctcttttcaactgtgtagtcccatacctacacagtctcagaatcatcccacttgaccttccccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactctgtatatatgaaataaattaattaatatttaaataatatgtattacttacagtgttacctaccatcacaaacaccaagtgggataaaagaattgagaaaaattgcattggaaaatttgagaggagatggaaaaggagagaggaagaagaatgaaagagtttatgattatgatgtgtataatgatctcggacaaccggacaacaatgatgacctaaaaagacctgttcttggcggatcaaaagaattcccttatcctaggcgttgtagaactggacggcctccaactgaaactg

SEQ ID NO: 14 - DNA, 440 bp, Cannabis sativa
atgtcttttatgatgagcatgaatccttctccctcctcgccaccgccaccgttatcgtcgccgtcggaatcttcctcaacgccgtcaacactgccagtccgtacgatcccgggaagctacggatggccgttactggggcccatctcggaccggttagactacttctggttccaaggcccagatacgtttttcagaaaaagagtagagaaatacaagagcacagtgttccgtaccaacatacccccgacctttcctttcttcagcgttaatccgaacattgtggccgtgctggactgtaaatcattttctcatcttttcgacatggaaattgtcgagaaaaagaatgttcttgttggagatttcatgcccagtgtcaattacactggtgatattagggttggagcttatctcgacacttctgaaccacaacacgctaaggt

SEQ ID NO: 15 - DNA, 2533 bp, Cannabis sativa
ttagatcaaataacccgtcccgaaatccaatatatatatatatataatattcaaactctctctttctatcttcgtacagtttaatagaagtaataatgggtaagaattacaagtccctggactctgttgtggcctctgacttcatagccctaggtatcacctctgaagttgctgagacactccatggtagactggccgagatcgtgtgtaattatggcgctgccactccccaaacatggatcaatattgccaaccatattctgtcgcctgacctccccttctccctgcaccagatgctcttctatggttgctataaagactttggacctgcccctcctgcttggatacccgacccggagaaagtaaagtccaccaatctgggcgcacttttggagaagcgaggaaaagagtttttgggagtcaagtataaggatcccatttcaagcttttctcatttccaagaattttctgtaagaaaccctgaggtgtattggagaacagtactaatggatgagatgaagataagtttttcaaaggatccagaatgtatattgcgtagagatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgactatcaaaagcaaaagccatttttacacaggatcatattattcgtgggaagaagcgtattcccttatacagtagagttgtggaagccaagtctcccatggccattgttattccttgtagtggctctaatattggtgcagaattgcgtgatggcgatatttcttgggattactttctagaaagagcaaaagagtttaaaaattgtgaatttactgctagagaacaaccagttgatgcctatacaaacatcctcttctcatctggaacaacaggggagccaaaggcaattccatggactcaagcaactcctttaaaagcagctgcagatgggtggagccatttggacattaggaaaggtgatgtcattgtttggcccactaatcttggttggatgatgggtccttggctggtctatgcttcactccttaatggggcttctattgccttgtataatggatcaccacttgtttctggctttgccaaatttgtgcaggatgctaaagtaacaatgctaggtgtggtccctagtattgttcgatcatggaaaagtaccaattgtgttagtggctatgattggtccaccatccgttgcttttcctcttctggtgaagcatctaatgtagatgaatacctatggttgatggggagagcaaactacaagcctgttatcgaaatgtgtggtggcacagaaattggtggtgcattttctgctggctctttcttacaagctcaatcattatcttcatttagttcacaatgtatgggttgcactttatacatacttgacaagaatggttatccaatgcctaaaaacaaaccaggaattggtgaattagcgcttggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaagattagttccatagagattgaacgagtttgtaatgaagttgatgacagagttttcgagacaactgctattggagtgccacctttgggcggtggacctgagcaattagtaattttctttgtattaaaagattcaaatgatacaactattgacttaaatcaattgaggttatctttcaacttgggtttacagaagaaactaaatcctctgttcaaggtcactcgtgttgtgcctctttcatcacttccgagaacagcaaccaacaagatcatgagaagggttttgcgccagcaattttctcactttgaatgaagtgttataatataatacatacatatatgctttgaataaagtgtagttgattacatagagcagtttgcattatcaaatgcactaataataagttaaatgctctaaaatatgtgttaaatcttatggcaggattatagatttgattaagatagtcatattgaaccataaattttcattcaacacatttttagtgcatttgacattatttttattgaatttaataaatactgactacattgtatagtccactacatcataaaaaaaaaaaaaaaaaa

SEQ ID NO: 16 - DNA, 1158 bp, Cannabis sativa
atgaatcatcttcgtgctgagggtccggcctccgttctcgccatcggcaccgccaatccggagaacattttaatacaagatgagtttcctgactactactttcgggtcaccaaaagtgaacacatgactcaactcaaagaaaagtttcgaaaaatatgtgacaaaagtatgataaggaaacgtaactgtttcttaaatgaagaacacctaaagcaaaacccaagattggtggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactcagtccctcagtgaagcgtgtgatgatgtatcaactaggctgttatggtggtggaaccgttctacgcattgccaaggacatagcagagaataacaaaggcgcacgagttctcgccgtgtgttgtgacataatggcttgcttgtttcgtgggccttcagattctgacctcgaattactagtgggacaagctatctttggtgatggggctgctgcggtgattgttggagccgaacccgatgagtcagttggcgaaaggccgatatttgagttagtgtcaactgggcaaacaatcttaccaaactcggaaggaactattgggggacatataagggaagcaggactgatatttgatttacataaggatgtgcctatgttgatctctaataatattgagaaatgtttgattgaggcatttactcctattgggattagtgattggaactctatattttggattacacacccaggtgggaaagctattttggacaaagtagaggagaagttgcatctaaagagtgataagtttgtggattcacgtcatgtgctgagtgagcatgggaatatgtctagctcaactgtcttgtttgttatggatgagttgaggaagaggtcgttggaggaagggaagtctaccactggagatggatttgagtggggtgttctttttgggtttggaccaggtttgactgtcgaaagagtggtcgtgcgtagtgttcccatcaaatattaa

SEQ ID NO: 17 - DNA, 485 bp, Cannabis sativa
aaaaaagaagaagaagaagaaagttgagaaagagaatggcagtgaagcatttgattgtattgaagttcaaagatgaaatcacagaagcccaaaaggaagaatttttcaagacgtatgtgaatcttgtgaatatcatcccagccatgaaagatgtatactggggtaaagatgtgactcaaaagaataaggaagaagggtacactcacatagttgaggtaacatttgagagtgtggagactattcaggactacattattcatcctgcccatgttggatttggagatgtctatcgttctttctgggaaaaacttctcatttttgactacacaccacgaaagtagactatatatagtagccgaccaagctgccttcatcttcatcttctcaaataatatatctaatatctaattatataataataactacttaataaaagactgtgtttataacattaaataataataataataaagtcttttgtagct

SEQ ID NO: 18 - DNA, 1635 bp, Cannabis sativa
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 19 - DNA, 1885 bp, Cannabis sativa
aaaaaaatcattaggactgaagaaaaatgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaattatctttaaatagatatatttcccttatcaattagttaatcattataccatacatacatttattgtatatagtttatctactcatattatgtatgctcccaagtatgaaaatctacattagaactgtgtagacaatcataagatatatttaataaaataaattgtctttcttatttcaatagcaaataaaataatattattttaaaaaaaaaaaaaaaaa

SEQ ID NO: 20 - DNA, 1635 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatgtaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 21 - DNA, 1188 bp, Cannabis sativa
atgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtctctgatgttcaaggcattcttttttttggtggctatattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctatatatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcatataa

SEQ ID NO: 22 - DNA, 20 bp, Artificial Sequence
  Synthetic sgRNA
aaggtacccggcattattca

SEQ ID NO: 23 - DNA, 20 bp, Artificial Sequence
  Synthetic sgRNA
gctgttggaagggatcttaa

SEQ ID NO: 24 - DNA, 20 bp, Artificial Sequence
  Synthetic sgRNA
catgtcggaatcaaggtacc

SEQ ID NO: 25 - DNA, 20 bp, Artificial Sequence
  Synthetic sgRNA
ataagcttgacctgctgtca

SEQ ID NO: 26 - DNA, 20 bp, Artificial Sequence
  Synthetic sgRNA
catacagtatcaaagacagg

SEQ ID NO: 27 - DNA, 20 bp, Artificial Sequence
  Synthetic sgRNA
tggggccatgactgcaggaa

SEQ ID NO: 28 - DNA, 20 bp, Artificial Sequence
  Synthetic sgRNA
agaaagacttctggtctagc

SEQ ID NO: 29 - DNA, 20 bp, Artificial Sequence
  Synthetic sgRNA
tacccgcataagattcttac

SEQ ID NO: 30 - DNA, 20 bp, Artificial Sequence
  Synthetic sgRNA
ccggtggatggacataatgt

SEQ ID NO: 31 - DNA, 20 bp, Artificial Sequence
  Synthetic sgRNA
gaggaatgataagtgcttct

SEQ ID NO: 32 - DNA, 20 bp, Artificial Sequence
  Synthetic sgRNA
tcaggtgctacttcagctgg

SEQ ID NO: 33 - DNA, 20 bp, Artificial Sequence
  Synthetic sgRNA
atacacgcagcaatgggtgg

SEQ ID NO: 34 - DNA, 20 bp, Artificial Sequence
  Synthetic sgRNA
acacagcttagggaagcggg

SEQ ID NO: 35 - DNA, 20 bp, Artificial Sequence
  Synthetic sgRNA
ctggacacagcttagggaag

SEQ ID NO: 36 - DNA, 20 bp, Artificial Sequence
  Synthetic sgRNA
gtggagcctagaattgaaac

SEQ ID NO: 37 - DNA, 20 bp, Artificial Sequence
  Synthetic sgRNA
agagtagtgggacttgcagc

SEQ ID NO: 38 - DNA, 20 bp, Artificial Sequence
  Synthetic sgRNA
ttcggcaagaagggttacat

SEQ ID NO: 39 - DNA, 20 bp, Artificial Sequence
  Synthetic sgRNA
gtgaactgattttgcggggt

SEQ ID NO: 40 - DNA, 20 bp, Artificial Sequence
  Synthetic sgRNA
tgggtgtttagtccatgagg

SEQ ID NO: 41 - DNA, 20 bp, Artificial Sequence
  Synthetic sgRNA
tcatgtgaactgattttgcg

SEQ ID NO: 42 - DNA, 20 bp, Artificial Sequence
  Synthetic sgRNA
actggagcagggctaggtgg

SEQ ID NO: 43 - DNA, 20 bp, Artificial Sequence
  Synthetic sgRNA
agaaagtgcccactggagca

SEQ ID NO: 44 - DNA, 20 bp, Artificial Sequence
  Synthetic sgRNA
acctctgttcctaaaaggat

SEQ ID NO: 45 - DNA, 20 bp, Artificial Sequence
  Synthetic sgRNA
gccttcatctctcaaaattg

SEQ ID NO: 46 - DNA, 20 bp, Artificial Sequence
  Synthetic sgRNA
cctctgttcctaaaaggatt

SEQ ID NO: 47 - DNA, 20 bp, Artificial Sequence
  Synthetic sgRNA
cgctgagcaagctccgtctg

SEQ ID NO: 48 - DNA, 20 bp, Artificial Sequence
  Synthetic sgRNA
gacggccatggcggcggcga

SEQ ID NO: 49 - DNA, 20 bp, Artificial Sequence
  Synthetic sgRNA
gtcgcggcggccgatacaga

SEQ ID NO: 50 - DNA, 20 bp, Artificial Sequence
  Synthetic sgRNA
ggagtagcagatacctcaga

SEQ ID NO: 51 - DNA, 20 bp, Artificial Sequence
  Synthetic sgRNA
ccttacacaaaactgttagg

SEQ ID NO: 52 - DNA, 20 bp, Artificial Sequence
  Synthetic sgRNA
gaggctttctgatttaaaga

SEQ ID NO: 53 - DNA, 20 bp, Artificial Sequence
  Synthetic sgRNA
ctaaaagttctgattttgtg

SEQ ID NO: 54 - DNA, 20 bp, Artificial Sequence
  Synthetic sgRNA
gatgacgacaaatgatacca

SEQ ID NO: 55 - DNA, 20 bp, Artificial Sequence
  Synthetic sgRNA
gaagtggatagtcccagccc

SEQ ID NO: 56 - DNA, 20 bp, Artificial Sequence
  Synthetic sgRNA
actcactgaaccgcccgagg

SEQ ID NO: 57 - DNA, 20 bp, Artificial Sequence
  Synthetic sgRNA
ctgggactatccacttcact

SEQ ID NO: 58 - DNA, 20 bp, Artificial Sequence
  Synthetic sgRNA
cacttgggagttactgaagc

SEQ ID NO: 59 - DNA, 20 bp, Artificial Sequence
  Synthetic sgRNA
ggcaatgtagagcttggcag

SEQ ID NO: 60 - DNA, 20 bp, Artificial Sequence
  Synthetic sgRNA
atgggagactctgccgacgc

SEQ ID NO: 61 - DNA, 20 bp, Artificial Sequence
  Synthetic sgRNA
cttgtctcctctcaccgcta

SEQ ID NO: 62 - DNA, 20 bp, Artificial Sequence
  Synthetic sgRNA
ccttgtctcctctcaccgct

SEQ ID NO: 63 - DNA, 20 bp, Artificial Sequence
  Synthetic sgRNA
tttctgcagatgcattctag

SEQ ID NO: 64 - DNA, 20 bp, Artificial Sequence
  Synthetic sgRNA
tgttccatgttcaaccaagg

SEQ ID NO: 65 - DNA, 20 bp, Artificial Sequence
  Synthetic sgRNA
ggcgaggagtgagtaacgca

SEQ ID NO: 66 - DNA, 20 bp, Artificial Sequence
  Synthetic sgRNA
caggctgagagacagagcat

SEQ ID NO: 67 - DNA, 20 bp, Artificial Sequence
  Synthetic sgRNA
tctacctccttggttgaaca

SEQ ID NO: 68 - DNA, 20 bp, Artificial Sequence
  Synthetic sgRNA
ggcatctacatcggctgttg

SEQ ID NO: 69 - DNA, 20 bp, Artificial Sequence
  Synthetic sgRNA
tttagcaccgtcattgtgcg

SEQ ID NO: 70 - DNA, 20 bp, Artificial Sequence
  Synthetic sgRNA
agcaccgtcattgtgcgtgg

SEQ ID NO: 71 - DNA, 20 bp, Artificial Sequence
  Synthetic sgRNA
ctaatccttgactcagataa

SEQ ID NO: 72 - DNA, 20 bp, Artificial Sequence
  Synthetic sgRNA
attaagacttgtctgagtat

SEQ ID NO: 73 - DNA, 20 bp, Artificial Sequence
  Synthetic sgRNA
taaccaattcaatgcagcaa

SEQ ID NO: 74 - DNA, 20 bp, Artificial Sequence
  Synthetic sgRNA
acaagaaagacgcacctggg

SEQ ID NO: 75 - DNA, 20 bp, Artificial Sequence
  Synthetic sgRNA
tatgagaagaccgtcgttgg

SEQ ID NO: 76 - DNA, 20 bp, Artificial Sequence
  Synthetic sgRNA
tgttatgccgtcagaagaga

SEQ ID NO: 77 - DNA, 20 bp, Artificial Sequence
  Synthetic sgRNA
ttgctgcattgaattggtta

SEQ ID NO: 78 - DNA, 20 bp, Artificial Sequence
  Synthetic sgRNA
ttcctggtcaagtgtctaat

SEQ ID NO: 79 - DNA, 20 bp, Artificial Sequence
  Synthetic sgRNA
acaaagctcgaagatatggg

SEQ ID NO: 80 - DNA, 20 bp, Artificial Sequence
  Synthetic sgRNA
gccacaaatagagcatcgac

SEQ ID NO: 81 - DNA, 20 bp, Artificial Sequence
  Synthetic sgRNA
aaggatcccaatcttgatag

SEQ ID NO: 82 - DNA, 20 bp, Artificial Sequence
  Synthetic sgRNA
gcgaattggagcggaacagg

SEQ ID NO: 83 - DNA, 20 bp, Artificial Sequence
  Synthetic sgRNA
cgatcccgggaagctacgga

SEQ ID NO: 84 - DNA, 20 bp, Artificial Sequence
  Synthetic sgRNA
gtagtctaaccggtccgaga

SEQ ID NO: 85 - DNA, 20 bp, Artificial Sequence
  Synthetic sgRNA
gcccagtgtcaattacactg

SEQ ID NO: 86 - DNA, 20 bp, Artificial Sequence
  Synthetic sgRNA
tactggggcccatctcggac

SEQ ID NO: 87 - DNA, 20 bp, Artificial Sequence
  Synthetic sgRNA
ggaccccagaccccagacac

SEQ ID NO: 88 - DNA, 20 bp, Artificial Sequence
  Synthetic sgRNA
cggacccagactccagacct

SEQ ID NO: 89 - DNA, 20 bp, Artificial Sequence
  Synthetic sgRNA
gtttcccgactactactttc

SEQ ID NO: 90 - DNA, 20 bp, Artificial Sequence
  Synthetic sgRNA
tcatcttcgtgctgagggtc

SEQ ID NO: 91 - DNA, 20 bp, Artificial Sequence
  Synthetic sgRNA
gtgcaaaggccatcaaagaa

SEQ ID NO: 92 - DNA, 20 bp, Artificial Sequence
  Synthetic sgRNA
gtctgcaccgggcatgtcag

SEQ ID NO: 93 - DNA, 20 bp, Artificial Sequence
  Synthetic sgRNA
ttaccagtatacatctttca

SEQ ID NO: 94 - DNA, 20 bp, Artificial Sequence
  Synthetic sgRNA
agtatacatctttcatggct

SEQ ID NO: 95 - DNA, 20 bp, Artificial Sequence
  Synthetic sgRNA
cagtatacatctttcatggc

SEQ ID NO: 96 - DNA, 20 bp, Artificial Sequence
  Synthetic sgRNA
tgaaatcacagaagcccaaa

SEQ ID NO: 97 - DNA, 20 bp, Artificial Sequence
  Synthetic sgRNA
attattcatcctgcccatgt

SEQ ID NO: 98 - DNA, 20 bp, Artificial Sequence
  Synthetic sgRNA
attgttcatccggcccatgt

SEQ ID NO: 99 - DNA, 20 bp, Artificial Sequence
  Synthetic sgRNA
agcataattgtggccctaac

SEQ ID NO: 100 - DNA, 20 bp, Artificial Sequence
  Synthetic sgRNA
gcataattgtggccctaact

SEQ ID NO: 101 - DNA, 20 bp, Artificial Sequence
  Synthetic sgRNA
agaaacaaaatgggagccgg

SEQ ID NO: 102 - DNA, 20 bp, Artificial Sequence
  Synthetic sgRNA
tttcagtgaccaccaatggg

SEQ ID NO: 103 - DNA, 20 bp, Artificial Sequence
  Synthetic sgRNA
ccactttattggatcttcca

SEQ ID NO: 104 - DNA, 20 bp, Artificial Sequence
  Synthetic sgRNA
gaaacaaaaatgggagccgg

SEQ ID NO: 105 - DNA, 20 bp, Artificial Sequence
  Synthetic sgRNA
atggctctacaaacactact

SEQ ID NO: 106 - DNA, 20 bp, Artificial Sequence
  Synthetic sgRNA
attggtggtcactgaaagag

SEQ ID NO: 107 - DNA, 20 bp, Artificial Sequence
  Synthetic sgRNA
ctagggagagtcctaacact

SEQ ID NO: 108 - DNA, 20 bp, Artificial Sequence
  Synthetic sgRNA
agtgttaggactctccctag

SEQ ID NO: 109 - DNA, 20 bp, Artificial Sequence
  Synthetic sgRNA
ggagtcaatggtgaaaacag

SEQ ID NO: 110 - DNA, 20 bp, Artificial Sequence
  Synthetic sgRNA
agagagcgttggaagcaatg

SEQ ID NO: 111 - DNA, 20 bp, Artificial Sequence
  Synthetic sgRNA
ctacaaatggattgatgaca

SEQ ID NO: 112 - DNA, 20 bp, Artificial Sequence
  Synthetic sgRNA
ccttgggagatccaataaag

SEQ ID NO: 113 - DNA, 20 bp, Artificial Sequence
  Synthetic sgRNA
atttcgcttagcgtgaatgg

SEQ ID NO: 114 - DNA, 20 bp, Artificial Sequence
  Synthetic sgRNA
agccttgggagatccaataa

SEQ ID NO: 115 - DNA, 20 bp, Artificial Sequence
  Synthetic sgRNA
gcttagcgtgaatggtggtt

SEQ ID NO: 116 - DNA, 20 bp, Artificial Sequence
  Synthetic sgRNA
ggcgcacttttggagaagcg

SEQ ID NO: 117 - DNA, 20 bp, Artificial Sequence
  Synthetic sgRNA
gccaatgcatgtggatgctg

SEQ ID NO: 118 - DNA, 20 bp, Artificial Sequence
  Synthetic sgRNA
attttctgtaagaaaccctg

SEQ ID NO: 119 - DNA, 20 bp, Artificial Sequence
  Synthetic sgRNA
ggtagtgaatggcttccagg

SEQ ID NO: 120 - DNA, 20 bp, Artificial Sequence
  Synthetic sgRNA
gaaggagtgacaataacgag

SEQ ID NO: 121 - DNA, 20 bp, Artificial Sequence
  Synthetic sgRNA
aggacataccctcagcatca

SEQ ID NO: 122 - DNA, 20 bp, Artificial Sequence
  Synthetic sgRNA
tcaagtctactacaacaaat

SEQ ID NO: 123 - DNA, 20 bp, Artificial Sequence
  Synthetic sgRNA
gacaataacgagtggttttg

SEQ ID NO: 124 - DNA, 20 bp, Artificial Sequence
  Synthetic sgRNA
tattgccctactgttggcgt

SEQ ID NO: 125 - DNA, 20 bp, Artificial Sequence
  Synthetic sgRNA
tcaagtctactatagcaaat

SEQ ID NO: 126 - DNA, 20 bp, Artificial Sequence
  Synthetic sgRNA
tattcatagccaaactgcgt

SEQ ID NO: 127 - DNA, 20 bp, Artificial Sequence
  Synthetic sgRNA
agacttgagaaacatgcata

SEQ ID NO: 128 - DNA, 20 bp, Artificial Sequence
  Synthetic sgRNA
atattcatagccaaactgcg

SEQ ID NO: 129 - DNA, 20 bp, Artificial Sequence
  Synthetic sgRNA
gtccacctacgccaacagta

SEQ ID NO: 130 - DNA, 20 bp, Artificial Sequence
  Synthetic sgRNA
gtccagctgcgctaacagta

SEQ ID NO: 131 - DNA, 20 bp, Artificial Sequence
  Synthetic sgRNA
tgtccagctgcgctaacagt

SEQ ID NO: 132 - DNA, 20 bp, Artificial Sequence
  Synthetic sgRNA
gcagctggacactttggtgg

SEQ ID NO: 133 - DNA, 20 bp, Artificial Sequence
  Synthetic sgRNA
tgttcatagccaaatcgcaa

SEQ ID NO: 134 - DNA, 20 bp, Artificial Sequence
  Synthetic sgRNA
tatagcggtgttgtaaatta

SEQ ID NO: 135 - DNA, 20 bp, Artificial Sequence
  Synthetic sgRNA
gttagatcagctgggcagaa

SEQ ID NO: 136 - DNA, 20 bp, Artificial Sequence
  Synthetic sgRNA
ggaatattacagataatcaa

SEQ ID NO: 137 - DNA, 20 bp, Artificial Sequence
  Synthetic sgRNA
gatactatcatcttctatag

SEQ ID NO: 138 - DNA, 20 bp, Artificial Sequence
  Synthetic sgRNA
gcgggtggacactttagtgg

SEQ ID NO: 139 - DNA, 20 bp, Artificial Sequence
  Synthetic sgRNA
tactgccctactgttggcgc

SEQ ID NO: 140 - DNA, 20 bp, Artificial Sequence
  Synthetic sgRNA
tgtccacccgcgccaacagt

SEQ ID NO: 141 - DNA, 20 bp, Artificial Sequence
  Synthetic sgRNA
gtactgccctactgttggcg

SEQ ID NO: 142 - DNA, 20 bp, Artificial Sequence
  Synthetic sgRNA
acagtagggcagtacccagc

SEQ ID NO: 143 - DNA, 20 bp, Artificial Sequence
  Synthetic sgRNA
gatgcgaaattatggcctcg

SEQ ID NO: 144 - DNA, 20 bp, Artificial Sequence
  Synthetic sgRNA
gctgggtactgccctactgt

SEQ ID NO: 145 - DNA, 20 bp, Artificial Sequence
  Synthetic sgRNA
ggtaaatctaagattttgta

SEQ ID NO: 146 - DNA, 20 bp, Artificial Sequence
  Synthetic sgRNA
tggaacatagaatagtgcct

SEQ ID NO: 147 - DNA, 20 bp, Artificial Sequence
  Synthetic sgRNA
ttttagatcgaaaatccatg

SEQ ID NO: 148 - DNA, 20 bp, Artificial Sequence
  Synthetic sgRNA
acagtaggacagtacccagc

SEQ ID NO: 149 - DNA, 20 bp, Artificial Sequence
  Synthetic sgRNA
tggagcatagaatagtgcct

SEQ ID NO: 150 - DNA, 20 bp, Artificial Sequence
  Synthetic sgRNA
tcaagtctactataacaaat

SEQ ID NO: 151 - DNA, 20 bp, Artificial Sequence
  Synthetic sgRNA
gagaatcttagttttcctgc

SEQ ID NO: 152 - DNA, 20 bp, Artificial Sequence
  Synthetic sgRNA
actttggaatcattgcagcg

SEQ ID NO: 153 - DNA, 20 bp, Artificial Sequence
  Synthetic sgRNA
acatgattccagctcgatga

SEQ ID NO: 154 - DNA, 20 bp, Artificial Sequence
  Synthetic sgRNA
tacatgattccagctcgatg

SEQ ID NO: 155 - DNA, 20 bp, Artificial Sequence
  Synthetic sgRNA
gttagtaaaagtaaaaacca

SEQ ID NO: 156 - DNA, 20 bp, Artificial Sequence
  Synthetic sgRNA
atttggggtgaaaagtattt

SEQ ID NO: 157 - DNA, 20 bp, Artificial Sequence
  Synthetic sgRNA
gtgctagatcgaaaatctat

SEQ ID NO: 158 - DNA, 20 bp, Artificial Sequence
  Synthetic sgRNA
gatctcttttgggctatacg

SEQ ID NO: 159 - DNA, 20 bp, Artificial Sequence
  Synthetic sgRNA
agtgctagatcgaaaatcta

SEQ ID NO: 160 - DNA, 20 bp, Artificial Sequence
  Synthetic sgRNA
gttagatcagctgggcagaa

SEQ ID NO: 161 - DNA, 20 bp, Artificial Sequence
  Synthetic sgRNA
ctaaacatagtagactttgt

SEQ ID NO: 162 - DNA, 20 bp, Artificial Sequence
  Synthetic sgRNA
taaacatagtagactttgtt

SEQ ID NO: 163 - DNA, 20 bp, Artificial Sequence
  Synthetic sgRNA
acaaatgcagattctggaat

SEQ ID NO: 164 - DNA, 372 bp, Cannabis sativa
catttgatgatagaattgcctcacaatattcgattctatcgattacttaccttggctacttctcttagttctctaagaggcctgtttgattgcagcctactgagagcactactcaaagctccaacaggtggtatgggcccatcaagagaggcagtaggtaaagaaacctgtttattgtcgttaagaataataatcatgtcggaatcaaggtacccggcattattcatggcttcataagcttgacctgctgtcatggcgccatcacctatgacagccactacattattctttcttcctttaagatcccttccaacagccattcctacaccaagatgaagaaatttaacatatgatattagtagctaacttaaa

SEQ ID NO: 165 - DNA, 2887 bp, Cannabis sativa
atttctttttgtgaaaaatgataattaagcttccgtgggaatgatttttcaggatcttgaacagctagcagcagagcttagagcagatgtagtccatacagtatcaaaaacaggtggtcatctgagtgcaagcttgggagttgtggagctcactgtagcactgcatcatgttttcaatacccctgatgataaaatcatatgggatgttggacatcaggtcaccacaacattaatgtctttatattaaaataattacaacccaattgagttattctgaaaaagaaatgaatttttgcagacatacccgcataagattcttacaggaaggaggtctcaaatgcataccattagaaagacttctggtctagcagggtttcccaaaagagatgagagtgtttacgatgctttcggtgcaggtcacagttctacaagcatatcagcaggccttggtatcttcatatttaactctaattattattattattattattagattaatttgtcgcataaatatttaagtttaaatgccagttgcaaataaatacttaaagttcaatttttgactaataatacttaaattattatttgagaacttttgtaggtatctatttgttaagtattaagtaaattttcacatgtcaatttttaattggtgcaagtcatcaaatttttaccttttttaaaaaaataatttaaatatactaataataaaatgacacgtggattaagtaatattaccgctaaaaattaaacttaggtatttatttgcaactgaaagttaaacttaagtattaagtatttatgccgtaaattaatcttattattattattattattattattattattattattattattattattattattattattattattatttaattttcaatccttataaattaaagatcaatgatggagaagagtgttcaattgtggtgacaggcatggcagttgccagggatcttctgggaaagaagaacagtgttgtttctgtgattggagatggggccatgactgcaggaatggcatatgaagccatgaataatgccggctacttggacgccaacttgattgttgtattaaacgacaataaacaagtttctttaccaactgctactcttgatggtcctgcaaccccagtgggagctctaagtggtgctttgactaagcttcaagcaagcaccaagttcagaaaacttcgcgaagctgcgaaagtaaaccttaaagccaacttatatgactttctattttgagtttttcttttattttattttacatctcgtgtcaagaactgatttacgaatttttatctttcatagaccatcacaaaacaaattggagggccagcacatgaagttgcagctaaagtagatgagtatgctagaggaatgataagtgcttctgggtcaacactctttgaggagcttgggttgtattatattggtccggtggatggacataatgttggagatttagtcaccatttttgagaaagtgaaatcaatgccagcgccaggaccagtcttgatccacatcgtcacagagaaagggaaaggctatcccccagctgaagtagcacctgataaaatgcatggtgagacaatagatatttagtttatgcacttttttttcttttgagttcttgtcaattagcagagtattaatccctcaatgatttcaggagttgtaaagtttgacccaacaacaggaaagcaatttaagtccaaatcgtcgacactttcatatactcaatactttgctgaatctctaataaaagaagctgaagaagatgacaagattgttgccatacacgcagcaatgggtggtggcactggtctcaattatttccagaagaaatttcctgatcgttgctttgatgtggggattgctgagcaacatgctgtcacgtttgcagctggattagctgcagagggtctcaaaccattctgtgccatatactcatcattcctgcaacgaggatatgatcaggtaacaaatgacaacaatattgtttatcaacttcaacactgatgagtttttttttttttgccactcatattcacaatctttgaaattatttgataaacacaagtggccatttaggactagtttcaacctgttttgtgtgtactagaaactttctagtggataaaagaactgaaggtgttttcattcaggttgcacatgatgtagaccttcaaaaattacctgtccgttttgcattggatagagctggcatggttggcgcagatgggcctacccactgcggtgcatttgatatcacctacatggcctgcttgcccaacatggttgtcatggctccatcagatgaggctgaacttatgcacatggtggccacagcaacagctatagatgacagacccagttgcttcaggtttccaaggggcaatggaattggagcaaagcttccagctaataataaaggaactatacttgaggtaagcatagatcattatagaagtcttaagaaaatattcagacttaaatattgagattcatctctataatttttattcttaaatagattggaaaaggcagaatattaatggaaggcagcagagtagctattttgggttatggttctattgttcagcaatgtgtggaagctgcaagcatattaaagaaacaagacatttcagtgacagtagctgatgcaagattttgcaaaccattggatacaaatctcataagacggttagccaacgagcatgaaatcctaatcactgccgaagaaggttctattggaggctttgggtctcatgtgtcacactttctaagcttaagtggacttcttgatgggtctttaaaggtagccttcttataaaagagaaaattcatcatagttgtaacaatattcc

SEQ ID NO: 166 - DNA, 4311 bp, Cannabis sativa
aaacggttacaaaaaataatttttttaaaaggatacagtttgaatggctctgaacttgttatgccctgctgaagtgaaggttctatccgttttggactccatcaagtccacctgcttccctaagctgtgtccaggttttgcttccattcttgatattctctatttggctactcagaaagtaggagggagattacataaaatcaaggctcttattttttttagtggggaagcattaagttatgagctcagctgaagagggatgagtggttttgtcaatgctttctgaaatatgacaaagaaaataaagagcaaaaatgtcaagctttgtgactttttttccttttcttttagtttgaagctattaacttcttgcttataccaaagaattcttgtcccaacaaactaaactgtgttttgtgtccaagttttcattgtttagtatagattttagaagtgattcttatgttgcttaaaacacgtaattgattcttatgttgcctatatttaatatgattattaccagcattatttgggtttgaaccccaatgagggtattatttatggtgttttgatgtagttgataccttagattgtctgtttttgtttatgctgctcccagactatgacagcttgagtttatgccattctcttattcgttttctttaggtggaagtagtttccatagaaaggattgcagagtaccacttagaagaagagttcattgttcggtgcagcaacctcctccagcttggccaggaagagctattccagaggaaggtctctgtaattggaatgtcccaaagcctgtttcaattctaggctccactggctctatagggactcaggtacaaaatcaaaccaaaagactaaggaaagaaaaagagtgaattttgtctgtgtgattaacctgggaatcatggtttaaaggtcctatctgaatgcccttagtcaaaaatagttcctgtttcagaatatgacattaaaggcctagtcataaactttatatgggaaaaggattcacaaaaacaatttgtaaagatttaagttgtgaaaaaaaaaagatgggtcctttcattaccccaatcatgttttagagttacttgtataaatatttttttagcaacaaatgaaacataacttttattagccaggtttctgtaacttgactccagtcagagctacctatttcaagagagtttgagttctctttcttcagttcaggtcttttttgtttgaaaacagaagacattttctgtatttgattttaacagtatttggtttcttgccttattattattatctcttcatacattccttgtaactagagtatgttgattttcagacactgaacattgtggcagagaatccagataaattcagagtagtgggacttgcagctggttccaatgtaacccttcttgccgaacaggtattctgttaacctgacctaaattcacagatcataactatgatcgttagtgtatgaagatttaaaggttcaaattcaaaaacttcccatatatatgttagtttcatcattttgacaggtgaagagattcaagcctcaaatagttgctcttagaaatgaatcattaattggtgaactaaaagaggccttagctgatgtggaagaaatgcccgaaattattcctggggaacaaggagtcattgaggtgatcttgtttctaacagtctgatgataaaatacatcttttcttctagattattattatttttttattattattaatgccattctatcagcataaatatttctaacagcataaatatttctatttttaggttgcccggcacccagatacagtcacagtggttacaggaatagtaggttgtgctggattaaaggtaagtccataaaggtttagatgcacactatttttatagtatgatatcgtatatatgtagaatcaactgaagtgttcttcctattagctttatatttttttaccttttttgaggaggtttatttaaaatgtggcaagcatatttctggtactattaccagttgaagtatatcttgctttttcttttttcttagcctacagttgctgcaattgaggcaggtaaatacatagctttagccaataaagagaccctgattgctggaggtccattcatccttcctctagctcacaagcataacataaaaattcttcctgccgattcagaacattcagcagtatttcaggtacatatacaaacgaatttcaatggatcttgtctaccctattcattatttctctcacatccattagactatgttgtcaatatgtgaagcattggtaaatgtcaggaccaatattttcatggcattgtcatcattgtgccacatgttttcactatagttcattagaagtgaaggttaacgaaatctgttttcttcagtgtatccagggtttgcctgatggtgcactacggcgtgtcattttgacagcatctgggggagctttcaggtgtgaaataagtattttaattgaatttggtagcggaatattaaaattgttttaggccgttgataggacacggactttactacgggctactgcatcagcatttaagttatatattgatttggtgctatgatttacattttgaagttaaaaaacttgcataaattatttttttaaaaaaacatgcttgtagattgtcttattttctttgaataagaaagttaaaacttaaaattccataatctcttgtctcgtgtacaagatctatgtgtattatccttttcaaaattctttggacatgattagtcattaaacaagtatctggttagactgccaattttggtgatttcaatttttgaacccaagctcatttgttaagattctgatttggtgttatattaaaactcctttatgacagagattggccagttgaaaagctaaaagaagttaaggttgctgatgctctgaaacatccaaactggccgggtatgggaaagaaagtcactattgattctgctacccttttcaacaaggtttgatacttgttttcattgttttctaatgtattttctctttcattttcaactgcgagcagtaaaaaaaaggtgtctttgttttttctttttatcaattctgaaacagggtctggaagtcattgaagcccattatctattcggagcagactatgacgatattgacattgtgattcacccagaagctattatacactctatgattgaaacacaggtataaagctaaaactagtagtcttggttcttcttttgctagtttgcaataggactgtaaattacaaaatggagagtctattatacaaaattacggtctcttcaatttgtcttgcaggattcttctgttctggctcagttagggtggcctgacatgcatataccgattctctatactatgtcatggccagacagaatatactgttctgaagcaactttacctcgacttgatctttgcaagtaatcatctttttccataatatttttccacaatctttttccgcatgttttatcgaaagatatgcctacagtaagccaaaattcagcttttacatgtaaatccaacaaagtctactgctgacctttatatctaatttgtgactttcagaaccaatgtgaactgaggattttcccccctttatcgacatccgtaattattcaataaattagcatatttctgggtttttttaaataaatttctcaagtgaaagtcatgtttattctttcctagatgcctttttcatttttattatttttagttcttttcctacaaaaaatttgggctgagtgtataaatggaatcgacattgatgcaggcttggttcgctgacctttaggagtcctgacaaccagaagtacccatccatagatcttgcctattctgctggacgtgctgggggcaccatgactggagttctcagtgcagccaatgagaaagctgtagagatgtttgttgatgagaagtaggttttctatttcatcatctttcattgataagattgtcacttcaaatcccaactttgcactatctatcaacacactgctatctgttatatcttcctcattccaaattatattccacccatgttttcaggataagttatcttgaaatcttcaaagttgttgagctaacatgcgacaagcatcggtcagagatggtgacttcaccttctctcgatgaaattatccactacgacttgtgggcacgcgagtatgcaactactactttgcagagttcttccaatccaaaacctgttccaacggcataagaggagacaacttttccattgttgaccagggcagcaaaactttat

SEQ ID NO: 167 - DNA, 4248 bp, Cannabis sativa
tattttcagattacaaagaatgtgaaagtacaacaaaaaatgaatatttatgcaacagccttcacagagttcatactcaatattctctccgcaagcaataaatcatctggagtagtaacctgcaaaagatttgaccactttaaatatttattttgaaagggaccactttaaacttgattactttttatagaattatagcttccatatgaacaacaaaagtgaaagtttagtaccttgatgttggtgtaagcaccttcagttatatatacaggatgtccaaggtgttccactatagacacatcatcagtcacttccagattttccctgtagacaagtttaatgcatactatatcaaaatttaaagagaaagtacattggtcattttacaatgtgtatgaggaatttcttctaaccaaagtaacctcactgttcaggcttgaaattaaattgagatggtggaagtcaattatctaacttttttttaagaggtggtgaaaagataagtggttacctatttacaagctcaaatccttccttgagcaactcgggtttgatcacctgtagaagacaattgataggcatccatgaacattgaccaatcagtattatttgaaatgatcttaaattacgaaatgaagcaagaagagaaaaaaaaaaaaaaaaaagaaacttaaaattaaaattaaaattaaaaacctggggtgtctgcatttcccaaagttttttcctctccaacgttttagttacaaaagatgcattgtttgcctgcaccataattattaaacaactttcaaaataaacaaggtgaaaatattaggggattttacatttaatatggaactcatgcaattaatgcctataaaacaagataggtagacataaatggttatttcaaaggcaggagctagatcttgctcctgaatgaaacacgtttataataatatcaaaactgaaatcggcctaaatccaatagttaaaacaactaatcatataaaggaaataaacatgggtttaacatgcattcactacctctttgattgtagctttgacaggaacaccaagtacagctgctccatgcaaccaaccatcttcaatgacctaatatgcaaaccagtaaacttattcagcctcttagagcagaataattcacggaatgtattcaaagttcaaactttgaagtaatgttttgagataatttgcattatgtataattataattataatttcttatgcttatagttggcttattaaatggacagtaagtcttctcaattaactttgaaacgtgcccccaacatgcctgcatatccacagatgcaactgagaatgtctcagatatagtgtcatagcatgcttgcatttccacgaatgcaaccaagaactttccagaatactctgagtgattctttagtgaatatgacgtgacatgtttcttacgcatcatttctcatatttgatttgggttgtcatcacataaaataagatttttctatgcaacgcaaattttacaagtaatggtcaaacaattccctgaatcaatcaggaaaatatgcaaatactattagcatgtacttgacaaggaaggaagaaagaaagtgatggtgcaggatttgatactgaagattcataacgttcttgtacattaaaaattaccagaataaacttaaaactaatccaatgagttataaagtgcataaaatatgttctcacacctttttaacttcttctgttgttaccaaaggtctagcagaatcgtgaatgcacacaagcttagagtttggatcaattgcctgtaaaaagaatatgcagcactactattaaaaggccatttaatgtagaataccccaagcataaaaccaacataaccaactgcaagagattaaataatatagccactagtacacgtcttaagatgcaattcagaactatatgttaaactggtaaaaatgaatataatatccagataagggaaacaaacataaagtagttatcggtgtatcttatgctaaagtgacatccaaatagaaagaaagcagggcaaagaaacactaatctgacaaatggaccaccgaaaatatatttggaaagaatgccaaatttaaaataggaatatggaaatcaaaaaattatgcacctgaagtccactataaactgaatcctgtctttcctttccaggcagagcgaatttaagtccaacatggatcttgtctttggaatctgaaacattacaagttgctaagtggctttagctccaatttacataaaaagaaaatacttttttacttttaaggaaaatgcatgtcacgaatatgaagccaattacagagagagagagagagagagagagagagagagagagagagagagagagagagagagagcaccttcaaagatatcctcgtaagagggatcacaaactacaacaatttcagtcacttcaatcattttagaaaaagtgtagaagctgcagggaaacaactcagaattatcctcaagaacacaataacaaatgaataaaataaaaggagaaagaatgcgcgaacaactcaacaattgtctaaatggtataaacactataaaggtctcaactaagccactaggtttggtcttctggtgaggggagttgggtattaagcacaattgagatgagaccaaggtttgaaccttggatctcgtagtgttgagagattcctagtattaattaagcaccatgtgtgccaatgatctaaacacgtctcatctaggaccatggaatctagataaataccacaaagaattaagaaagttacttttacttccaacaagtcataaaaactcattacgttcacaacattagtgtgctggaccaaactcaaagctcaaatgatacattttgattcattattttaatgataaatatcaatgtgaaatcattacacaacacaattgttgcatgttctatggtttggctgattttttttctcttgactatttcaaatccgaagttttaacttttaaaagttaaactaaaggcagtattaccatacgaacacgacttcatttcgaaattcgtaaaaagaaagctgaagtatcatctactagtgcaaattgaaagtaaaatagagtttaagtgaatttacctatacagtgcaataggttgccctaaaagtggaagatactgctttggcatgttggcctattgtacaagataacaaaaccatacgatggtaggtgagaaatcaacagcctgcatgcaatagggacttacgagacatatgggaaataaccagaaatatataacaactatattacaagacatccttgctcaaaggaaactgcttttctacaatagcagtggagagttaagacaaatgtatgacccttctaattttgtgataaaatgtttcttaatctctcctgatacaataacaattttcaggaaatggaatataatagtaaattgaattgaattgagttaatacaataacacagatcaggtctcaagtgaaatgggtcatagaggaaaacgagcaggacttacccccatccgtttacccttcccacctgccagaagaaccaccgaaacgcttttcttttccacaacaccaggcttctacattgttatatgtatatataatataaataatagaacctattacgacaaagagaatacatttttatatctgatagaaccataaatcattgtttcgaaaagaataaattttgataagtcatagaattcgtatgtctgaatcttacttggccaacatttgcagagcagcgagcgaaccttatcttggaaattctgcatttccccctgaatcccacctcatggactaaacacccaagaagataaaaaggaaaaaagagaagaaaaagtgatcgtaaatacacttccagacaaaaatgagagtgcaaaataagcgaagtttggtggcataataaaaacaacccaattcccaattcgtgttatgatgaaaacagtgatcatgtgaactgattttgcggggttggagaagaagcttgaaatacttgcctgggaatcgaacaatggattgagaggatgggcgaatagaaggaaatagtagaggagcagcattgtaggaagaagaagaaagagaagcagaagagaaagtaaggtccattgcaagtaacgccatgttgcgtttttctctactgactttttttcttttcggaaatggagaaaagtgttataataaattaaggtatggaagttgggcagccacacacacacaagcatttaagggc

SEQ ID NO: 168 - DNA, 4029 bp, Cannabis sativa
tatggtcattttactacacgcattagtattattatgattcattcttattactcgacagaggaggaaaaattacgagagaaatcatctgaagggctacaagcgctagctgaagcaggttctttgtaccattcatttgcttctcgagtcagaaagttggcctctgcgtataacaaaacaactttatacatcttcattttttagttatttatttttttaattttttttttttgagggaaataaaaatcagtagctagagaaattaaattaaaggttgacttgcgtatctctaattgttctcttctaattctcagtggcttcactatgggaaaaataaccatgagactgaatggtctttgaaaatcatagaagattataaccagtctggtcttgcaaataacacatgactaccatcaatataaccttaattcattttcaattcgggttataaaaaatcctaaaacaagaatctcttaacttggtatttccaactttgatatttcataatttggataagtactcgaaattagtactcaggagctctttaatttttcactttgcagtgtgtaagacaaaataatatagtatagcactttcaaccacgaccttccttatgagaggttagatcactaaaattactacagatagccatgacaaaatatccgcattcatttggtagatttccacaactagaaaaaaaaaactaggtaaaaagcttagaagactaacctgacaaaaacacatccttgtagtcctcatcatcatatataaactgaggtggatctggggaaccgaccccgacaatggtgcttccactgcagcatgcacgatgagaaaatgtaaaattcttgtgacagatgatacaatgaaactacataacaagtccctcattcataaaattcattgatgcaagcatcttgatgaagattatttatgaacccttatatacagaaggacttgcaatcaatatcatcagatgattttttttgtttgtttctatagttacatggtcactaactaaatcttctcttaagaaatctaaaccttattgctcaattttaacagaaatatttgaaatttccagatgctaactgcacgatttagttataagcttagtaggaaaatgatcatccggaaagaaaaatcataacaggagacagatactctacaacagtatggccaaatggtcaggtcaggagaagtcagtttcttacttacccggttttggatacccaaagagccagatgttcctattgactttactttctaataaaattcatattgtgaaatgcagtttaaacatcccttatttataccatctgaacccatatacagtttaaaatccatagctcaaaatttccacattcagcatatgaacatatgagcaacataaagccataagagctagaaatccagtatttatacctcccagacataaaaacagcatcatattgtccacgactagctgcaattatacgctgtttcaatctcttaagggatggaagaacttcaaaggcaggaggttctgcaaataaaaatatcattagacatgtaaagaaacaaggaatacttctaagaatgcacatcgaagttggaaacttgttgagagagactcattaacaccaacgcccttttggaaaagcaatactggcatacgaacccagtacaaactcaagttagagtaaaaaagaatctaagcagtaaaactaaaatattttcttgacctccaatgtgtgttgagatatttaaaacatgaagcttcgacaatcaggtacataaaatattcaaatattcatgaccatccaaatattcatagcagcatatcctgtaataggtttattgcagtgtagtatcacaaatgaaaagggaatatttacttacctaagtcattgatgcagacatcttgacttattccatcacttgagatcttgtggagcaattgtaaaggatcactattactggttttatccaaccgaaaacgctgcaatacacaaaaaagttgccattaaaatgtaacaaaagaaaacgatgaaatattcaccatgttaaaagagcattaccttataaacttcggctgttgaacatgcttctgggggctttatgagaaccatgggaatgtttaatggtacaggtggtagaatatcctgaacaacctggacaaccaaagaacattttatagcagaattcatcacaattactagacctctatgcgaactcctttatttagattagacaagcatacataggtaaacaaataaaaaacataatgtcagattcaaactcatagactgtcactactgaaaaccctatgtagttatgcagtagtaacattaaacacaagaacccatgacatacagaattatattgcacacactgcacagctatatgcatctacacacaaattcctaggctgtatactgtgttccctcattttgactggttctgaaagccgatgcaacttctcaaatatgagaaaaaagagctgagagaaaagtacaatagggctttccatttaataaaaaagacagtagccaaattatggattttttttgaaactggctgcttgctttaccctacaatatttgagcatgaaactatggcaattggtacccaacagggattcaaattatggaaactttagcagattcatctttggcaatcacagaaaaaggattaatagattttcaaatgaatattgagttgtaaaagaattataatatctttgtttgaactacagacagtaagataaagacctacctcacctcgacctgtacaataggctgccccttgggagaaaaagaaaggaacatctgaaccaatctcacttgaccattcttgcaattccttttcagtaacaagacaatcattgaactgatttgctgcccatagtgctgttgcagcattgctgctcccaccacctagccctgctccagtgggcactttcttgtcaagatgaatctgccaacatgttaaatgacacaaggtatcatcaatcaatggcagctttctttaaaaacaaaacacaagacataaaactgttttaggggtctcttacccaaaagtatttgtttgtaccagttttctttcggtaaagattaagggccttgataatctgtacaaaagaaaaaaagtcctcttatgagtgattaaaaacatcatcatgcaacaaataaaaagcagcaaaggaacattcatcccttaccaaatttctatcatcaagtggtaccccagaggcattcgttgacaaagaatctttctttgttgaaggagacaaagagaacttaagcacatctccaagacttatcacctaaaatttcaaccaaataatcacatatgtttcatatattttccaacaacttgaacaaatttaagtaaaacttacgtgaaagagggatgccaaatcatgatacccatcttcccttttgctagttattctcaagaaaacattaatctgcattcatgatgagacatcaaaaaactgctgaaattagcataaatcaagaaacccatttactaaaaacaatgacaaaccttgcaaggtgagaaaagagtgagcctagaaagaggcgcttccttgtccactacatcagccaatttgtttatcctttcatcagcatcataaactatctacaaccaccaaactaataattaaaaaaaggatacaaatttatacacaaaaggtaataaatgatcaaaaagaataatctttacaagataaagactgaaaattttgaagtcaaaagggtgagaaggaatgggagattccaaaacaaacctcaacttgtcttctttcagaaaccacaattttgagagatgaaggcctcgaaattctagatttcgaaccaaaaaaacaaaacccatttgagtttaaggaagagagacccctgttcctaaaaggattgggagaaagattaaaaacgttgttgtggcagagaatatgagaggaagccattttagttgtggagaggtaagtatgaactaactaactatcaaactgtgaaaaaatggtgcatttaaagacttgggattcag

SEQ ID NO: 169 - DNA, 1944 bp, Cannabis sativa
actactaaccacatgtttttgacaaggactttccaagcatattcttttctactttttcataagaagaaccacagtgtgagcagcgatgcttcgattttccccgagactatcgaccttttcatgagttttcgctttcagattaacaactgcagggtcagctcctagaagttcagacaagttggctctgatagcttccttgtgtggacttaactttggtctttgaagaattaatgttgcatctaaatttccaagctcataacctgcctcatgcatcagtctcacctaaaatatagataaaacatattcaaacatgtttatattgtctctcaactactaatagacagctcagagtgaatggttttcaataattgtgttccattgtttgtgacaaagagacattgttgagaaaaacttcccaagtataattctaaatgataatttcaacaagtgtgtgtgcgtggatgtaaattctacagttaacaattcattattggcttcaaacaaaaagtcagacctagacagataaataagactttgtcaagggttaatcccaatcccaacagtctcaatcatttgggtttgtgttgaaaattgataaaactaaatatcaaatacttagttaacattatgaaaagagaaagaaaaacatcatttcttgcttagagtttatgttatagacagtggactaggcaaacctagctttgctgaaccactatatttcctttgtgtcattactttttactttctttttcattgcttattatttgagtattcgtactttaagcgagtaatcgccgttttggtgtgattgttcttgccttgctcgaatactcgcacttttagtgaaagatagaaaaacattatttcttgcttagagtttatgtttgaacagctaaaaggaaacatagcacaaactcaattcaatggtaaaaatctgtatatgactgaagatttcataagcacaatacaattcaatcaagtgtaaaatgcatagaaaaatcaaacaaaattggttgaatgaaacataacaataacaacttacagcttctttgatgaaaactgatgatgcagcccctttccatttgggatcagaatcagggaaaatttgaccaatatcaggaagccccaaagcacccaaaatagcatcaactacacaatgaagcaaaacatcccctacaaacccaaatcaaagaatatatctttaactctcttttcacaaagaaaatctaagaaagttcaagtttttatattctcagcaaccaaacagtataaaatttaagagttcttctacactgcacctcttagtgttaaacttgcaacagactatttgacgagttaaattattttgaatttctataaattataccaaatataagattaaaactatctaaatatggaaaacaaagagtcgaaatttctgtaaatttagagcttttactaacttaatccaaaaattaaaagaaacccatttcagatacgaatgattaccatcagaatgagcttcgcaacctttctcatgaggtatattaatacctccaattatcaaaggatacccaggctccaatcgatgaaggtcaaacccatgaccaacccgaaaaggaagagcctttgagggagtagcagatacctcagacggagcttgctcagcgttcaaagcggtggtcgcggcggccgatacagaaggcagagccgaaagcggcgtcgttttcgattgtcttagagatgaaattgaggacgatgagaagagatttctgggaactgcgaccgtgaaatgggagtgattgaatgatttgggggaggtattggagtagtgtggaggaagagttgaagcacagagtggtgttgccgtcgccgccgccatggccgtcgaaggtggaggtccgtgggtttgtggtttggctttctcttcttagctagttgttaattttttttttttttttaaaa

SEQ ID NO: 170 - DNA, 5363 bp, Cannabis sativa
ttttcttagctatcatattgagcttgtgttatggaattttgaaggtgagcttttgagtttgtggaaatggcttctggagctgtaccagcatcaatttcatgtctgaaaagcagagactctgccttgagctttgctaaaagttctgattttgtgaggctttctgatttaaagagggttggttcatctagaacaagagtttcagttatccgaaattcaaatcctggttcagatattgctgaacttcagcctgcatcaaaaggaagccctctattaggtactaatttctttgatctttcagtctttgttgattatttaaactcatgaggccttagaagaacaacagaacatttgtgagaagaggatatatctgcagaaatctaagcttaataactgactttttaagtgtttgttatgttgaaaatggaagtaataaacaacttcgaaataaattcggtaggtctgtgtttgttgcttgttttgtttttctgatgaaaaaatcgtttggatgtagttcctagacagaaatactgtgagtccttacacaaaactgttaggaggaaaacgcgaactgtgatggtgggaaatgtggctcttggcagtgagcatcccataagaattcaaacgatgacgacaaatgataccaaggatgttgctggaacagttgaagaggttttataatttctctaatcgaaaaatattacaattattcattggctcaactgcacaagcataagtttgatactaattagaatttattagtatgacatttagctcacttttgaaggggtagttagttagttcttgttaatccatttttttttttctattttttttttggtggtggtggtgaattcttctggaatttcgggttagtagaaacatgagtataacgaattttagaagagtttaaccttgtcaatagttcaaaggtgggaagtaatcactttttatatagaggcggtttacattactgtgtctattcaatttataggtgatgaggatagctgataagggagctgatattgttcggataacagttcagggaagaaaagaagcagatgcttgtttcgaaataaaaaattcacttgtgcagaagaagtaagttatgtttattctttgcttattggttttcgaaactttaaggtacatataatttgaaggattgattatgttactatcccatttgtatgtgcagttataatatccctcttgtggcagatattcattttgctcccccagttgcattaagagttgctgaatgcttcgataaaattcgtgtcaatcctggaaatttcggtaaaggctttgtgattcagttgatttatttgtgtagtaaataaaggcatttcttctggattcgtctaagcagtttgttacagctgacagacgggctcagtttgagacgcttgagtacacagacgacgactatcagaaagaacttgagcatattgagcaggttaaatccattcattatcgatttgtgatcattgtttatgtaaataaatcatttgtccataaattcattgaagctttcttttagcatacatgtgattgccccgattgaacttggaaaataaaagacgatatatatttttcattgtttatgtgttgtttctatttctgaaacaaaatcggttctttctcacttcacaccaggttttttctccattggttgagaaatgtaagaaatatggtagagcaatgcgtatcgggacaaatcatgggagtctttcagatcgtatcatgagctactatggagattctcctaggggaatggtaaattttttttttttcttattcttgtttttcttgtagaataaaatttaatggtgatacttgacttggcattcaccatcgaacaggttgaatctgcatttgagtttgcaaggatttgccggaagttggatttccataattttgtgttttcgatgaaagcaagcaacccagttgtcatggttcaggcgtatcgtctacttgttgctgaaatgtatgtccagggctgggactatccacttcacttgggagttactgaagcgggagaaggtgaggatggacgaatgaaatctgcaattggcatcgggacccttcttcaggtacctttctatttctgattcacgatcaacggagtcggtacacagatatttctgcacttcaatatgtcacgaactttaaagccatcggaattctttgattttggtcggttgtcttcctaaaagccatcactatgtttcgaacgcattttacaatttataattaagcaagatgcatgtcatgcatatcaccatagctggtgtaattgaatgttgaattgaatttgcaggatggtttgggtgatactatcagggtttcactcactgaaccgcccgaggaggaaattgatccctgcagaaggttggccaatttgggtacaaaagcagctgatcttcagcaaggagtggtaagatcttcaaatcccttcatttaattaaagacttctcttcaaatcaatatttagtacactatctcaactttctcgataatatgatttcgcttcaggctccatttgaagagaagcacaggcattattttgattttcaacgacgaactggtcaactgcctctacagaaggaggtcggcctttaatatcgagagattcgcatggcatattgctcaaagtttaaaactttttaacagaaattgatgcttcggttttacagggcgatgaggttgactatagaggtgctctgcaccgtgatggttctgttctcatgtcagtgtctctcaataacttaaaggtagctcacaaaagcacaacatccttccatttcgattttctttcttttgtcctcatttcgggcacgggagagaccattctagaaacccgaaaatataactgtttgtcccttttccagatgcccgagctactatacaggtctctagcagcaaagcttgtcgtcgggatgccatttaaggtctatatattttccattttataaccgtgtgaaatcgttatttcgcaaagctgaatatccatttgataagttttcgtttaattgattataggatctggcaacagtagattccatcttattgagacaacttccacctgttgacgatgacaacgctgtaagaaatataccgaactgcattttttcgtattagacaaagtcatatgatctgtgttctaattttcttcgcgtcttaatatgtttttgcattgttgttttaatcgatcagcgattagctctcaaaagattgatagacataagtatgggggtcataactcctttgtcggagcagctaacaaagccattgccaaatgctatggttttggtaaatcttaaggagttatcatctggtgcacacaagcttttgccagaaggtaaaacttttggattatttagctagagaagaaagatgtatatactaattctgactaatacatatataccgattttgttccaggcacgcgtttggttgtatccttgcgcggtgatgaaccttacgaagaactggagattctcaaaggcgttgatgatgttattatgattcttcatgatcttccgttcgatgaacataaaattagcagagtccactcagcaagaaggtaagtaattataagttagtgtggcttgtgattttgaaaccgctgcttttcaaggagattagactctgtaatcatacaattatttacttattagtacttttagttatgaaaaaaaaacttaggttgcgttcggtcggaaggaatgaatacatcagaatagaattagtaataagaatgagaatgagaataaaagtaggaatttgaatctagtccgggtttgggctatggtagtccgcggttagggtctacctaatccaatagtcccaaaaaaatatatattttttaaaaagtataccatattttttattggttttcgaaaatactacatttcattaatacaaggcatagtaatttttcttttgaaaatattttacaaataaacaaaaataacaaaaaattataaacatgcggttgtatagattgattttatttacagaaaatacggtcttttgatgtattttcatgttgtactcttgttaatctgttattgagtttttgttatttgtatgttattttttgttgttgttttgatgttatttttctgttacttttatatagttttcttattgttttagtgttgtctttataaaacactgtaaaaatgtaaaaaaaaaatctttgaacgtaaaaatgtaataaatttacaaaaaaaaaatgtatattatgtaattatccattttttttttctcaaggcacgtccaatctcagggtgaccgtagaataaaatttaatatgcataacaaaagtcaataattattttttcatcttgcattagaatgaccttactcgcacaagattattttatttaatttcttatattaacacacaatatgttactttatattggctgcagattatttgagtatctatcagataattctcttaactttcctgtaatacaccacattcaatttccaaatggaatccacaggtatgaattgaattctcttaatttgactatgaaagattatgagatattctattcagtcatgaattttgacttcaaaaccgttttcacgcagggatgacttagtcatcggtgcaggtagcaacgctggtgcccttttagtagatggactcggggacggtatcctcttagaagccccagatcaggatttcgattttcttagaaatacttctttcaacctacttcaaggttgtagaatgcgaaatacaaagacggtaagaagcttttatcatttgaaaggattcgaaatatatttcggaagaaaaaaaggtccaacttttctaaaatgtcgtcgcaggagtatgtctcgtgcccatcctgtggtagaactttgtttgaccttcaagaaattagtgcagagattcgagagaagacatcacacctgcccggtgtctcagtaagtactcgagtccttcgtttttttgaaatattattcctttgtatttaactcttttcgatcttttcgtatgatagattgcaatcatgggttgcattgttaatggacccggagagatggctgatgcagacttcggttatgtcggtggtgctcccggaaagattgacctttatgttggaaaggtatataattatgttttcggttttatatttcgaaagaacatatggttaaaatcgacatggaaatttacgttatgttttttcagacggtagtgcagcgtggaatcgcaatggaacaagcgaccgatgcattgattcagctaataaaagatcatggccgatgggttgaaccaccctcggacgaagaatgaagagtgtatcatcgtcgtcggtagatagaaaataaaagggcag

SEQ ID NO: 171 - DNA, 2307 bp, Cannabis sativa
gtcatccgatgcgccggcgagtcatcttcaacggcagcagattctgatttcgatgcgaaagtgttccgtaagaacttggtccgaagcaagaactacaatcggaaaggttttggccataaggaagagacccttcaactcatggacagcgagtacaccagtatgttgttgtgcttcgattcctcgatttgtttatgattaaaggatttttttttttgtttgcttaattgaataattaatttacttgaaacaaaattcaggtgatattataaagactttgaaggataatggaaatgagtacaggtgggggaacgtgacggtaaaattggccgaagcatatgggttttgctggggtgtggagcgagctgtccaaattgcttacgaagcaaggaaacagttccccgaagaaaagatttggattacaaacgaaattattcataatccgacagtcaacaaggtttgatttttttttttaattagaatttgtgcttcgtcttcgtaatggctcaatttatatgtttatttattgttattatttggatagagactagaggaaatgaaagtggaaaatattccaattgatgaagggaggaaacaatttgagattgtaaacaagggtgatgttgtgatattgcctgcttttggtgctggagtggatgagatgttggctttgagtgataggaatgttcaaattgttgataccacatgcccatgggtttccaaggtaacctttttgtttaataatagagatttttgaattatgggattctgtttaaattttatgttattaatgggtttggttacaaatcttctgtttgtaaggtttggaatacagtcgagaaacataagaaaggtgaatacacttccattattcatggtaaatatgctcatgaggagactatagctactgcatcttttgctggaacttacattattgtaaagaacatgaaagaggtaaacaactctcttatgacaaacaagttttatgctttagattttaaatgaaacctttatggtcttcaattcaggcaatgtatgtctgtgattatattcttggcggtcaacttgatggatccagctcaacaagagaggagtttatggaggtgtgttttgcctattcactttgattacttagttgataattttcttgacacaattttttcaatagttttataacattcgttcttgggctttgtctgcagaaatttaagaatgcagtttctaagggatttgatcctgacaaacatcttgtgaaggctggtattgcaaatcagactacaatgctcaagggggaaaccgaagagattggttagtttttgcagctggttatgctatagatcttgaagaattgccactacttgtttgcaatgtcttttttaacttatattctttaatttcagggaaactggttgagaggactatgatgcaaaagtacggagttgaaaacattaatgaacacttccaaagctttaacacaatttgcgatgcaacccaagtaataagttcctttctcttcagttcatgtattcttgtccggtttgttacttctgagatttcattttcctcgagcaaaacattttactgtcaaatgaccattttgttttctttgtttgtttgttcttctgattaattgacggcattcctcttgtgtcatgtagtctgagttaatttttttatgtttttctttaggagcgtcaagatgcaatgtacaagatggtggaggaacgtattgaccttatgttagttgttggaggatggaactctagtaacacttctcatctacaagagattgcagaggaacgaggtattccctcgtattggattgacagtgaacagagaataggtcctggaaacaagatagcctacaagctaaatgtaagtttggacagataagactaccattgcaatctaattatttcttgttttctgcttatgtactagtttagcagaactcctaagcaatggttccttctaatctgttgtcctgattgttgtaaattccttattgtgaattttctggtagcatggagagttggttgagaaagagaactggttaccagagggtcccatcacggtcggtgtaacatcaggtgcttctactccagataaggtgagttctttataccaatcaaaaatagcaaaccgcttcaatgaaatcataaaaagtcatctccattctactcacaaggaattttctttatcaggttgtggaagatgttctcatcaaggtgtttgaccttaagagcgaagaagctttgcaagttgcttagtttaagttagactcagtgtttatacatcaacagacagcgc

SEQ ID NO: 172 - DNA, 2919 bp, Cannabis sativa
ctttattggatagaaagacaattgcgccactaaatgactactaaggctattaagtcaacttgtgaatggttttcatatcagcaacttccttaagtgtgcctttctcaacatggtcccaccacttgaacaagaaattatccacaaccagtctgaaccaaggggatagcttcaagcctccttccccagcatctgctttcctcaacaactctttcaactcgtcccggttttacatacttgatatcagctacttcatctggatttggattgacactaacatcccggacgatgaagagcaggtaatcaactgccaaacaatggaataataaccaagaagtgagcatgacaagtcaaaaaaacagcgctcccttaatcactctcttaaacttaacaaatttcaacctggtgccaatagctcacaaatatctggatgctatttatttatttatttattttgcatatgatgtataataaagtaagaattattacgttcatgctcgccccatttgccatcagaaggagctttgtacagcatcctgcctagtggggtaaattgatcaactggcacatcttcagcaggaatacccagctcatctaaaagctttctctgtgctgcattccttgctcctggtaattaaaaaatataatgagattttgaagaggtaagctttcattggataatcaaatatatattctgcagaaatatattaccaagggactcctcatcgataagctcagattcacggtagagcgggtggctacaacaggtgtttgtccacacaagagggaatgttacctttgttgcagaacgttgctgtacagatgaacagatagaatatcatctcttgacacttatgttatcgaatagatgtgcataatagaaacaaactgggcttcaatataactagacacacacaaggagttttagtgaaagagaagaaacatagaaaacaagaaaagatgtagacttggtcatgatgtatatgttgattaaaaggacaaacagagactaatgtgcttaagcaaattatggccgataaaaaagaaaagcaaccctgcatacagaaaaatagaactatgacacacttattcaatttatgccctcctcataggatataaaggctgcataggtaatataggccggatcttgactctgttcttcaattgacacttatatttgctgtataagcacctaggagggtagttcgaaaagtcaggtctgtagtttgctttgctcccgcaagatctaacgtttggatacttaaatagaaaagcctatagtttgatgatcctcaaatattgcaatcaatacctgaagaagcaactcatattttgagttgaacaagaacacactgaaagccctgtgtagcaaattatccttttcaatcttttccatcaagtgacctacaagagacaaaagctttagcaaaccacggaggtctagctgatccaacagaatcaacatgatctattaataaaatatatggtgtcagagagagaggggggaaaaaatgtagaatgcccaaaacatgaataaacagttgatttccataacataaaggtatccaaaatcaaaagagtgtataagatttactgatttaggaaggatagaaagacttggagtatgcaaagtgccataaaaccgtgaaataatacccaaaaggaagtgttgtgcagatcaaattttacttacagttatattttgtatcatgaccaacaactcggtcattctcatccactagaatgcatctgcagaaagttcacacaaagaaacctaatcaactccagacaagcgaaaaatctagataaacagcccaaacgataaatatgtagatctaaaacccaaatttgcatttggattttcaatgttttccaattatagaaccaaatatttggctcaaaaaatatctcaactcatttgataatagttaataatctatttaagtctcacctcagccgctgaacattcatttcttacaaaaacaacaaacaacaaagaaatgaaatccgaggtttaattttatcaacctttgaactagcaaacagagcaagagaagggaaaacaaagccaataattcactttgttgtgttcaaaaacaaaacaagcataatacatatccgtcgatataagatttgaagttaatatcttcatgctttccaacaacaccaatagttcagttcatttggttgaaatgaaaaggtattgaaggggcactgttaatagaaccaaagacaaaattaagcaaacaaaacatgaaataaatcatgctaattacgcaagatttaggaagaaaaaaaaattatgctccttcaacaatacacacacacatataatatatatatacatatatatttcgtttccttaaagaatcaaaaagagggagaacatcaacaaacaaagtataatggttgaaatataacaataaagaacaaaatacaaaccaacccatgaaataagcaacccaaaaaatcaagaaaaacaaaatcaacatttgaatatatatatatttcctattcaaaatctaagcaaccaaacaaagcatatcagaagagtaacaaagaagaaatggaagaagggttatcagataaacagaaacgcactcatcatcaaacataaggcgtctctggacagcgtccattccagcgtcggcagagtctcccatggtggaagaaagagagaccctagcggtgagaggagacaagggcttaagaagggaggggattcggagaaaagggctacgagcaaaggtagaggaagaggtaaagggaagagttctgggggcaatgtagagcttggcagtggtatttagaaggagggtcattgtcgcatgtgatctccgccattgttgaatcgagtaatattataattatatattaatattttaatgtaactattaaatatgtgcctaatattatttaatt

SEQ ID NO: 173 - DNA, 1279 bp, Cannabis sativa
gagcagcagcactaattattattgtactaataaaatattaaatgatatcaattttgcctataagcaatataattagccaaagcaattaaaggagcagcctttatgggatcaaaaccagaaagatgctgtttggcttctgtgttcaattgctcagcaaattctcttgatttgtcaatacccattagccttggataagtaaccttatcagccaccaaatctttcccagcagttttacccaattcttgagaagatttagtcacatcaagaatgtcatcaacaacctgaaaaagcaagccaatacacctagcaaagctcctaagtttttcaacttcttcatctgtaccaccaccaagaatagcccccaaaacaacagaagcttctagaagagccccagtcttatggagatgaatgaactcaagttgttcaagcccaacattagccaaaccctctgaatcaatatcaaccacttgaccagccactaatccttctgacccaattgacttagcaagctcaccaatcgccctgacaatcttggctgccggaacaccaacggtagagaccgccatgtgctcaaaagcaaaggctaaaagtgcatcgccggctaaaacggccacgtcttctccgaagactttgtggtttgtgggctttccacggcggaggtcatcgttgtccatacaagggaggtcgtcgtggattagagacatggtgtggatcatttcgacggcgcaggcagccggcatggctacggattcttttccgcctacgagttcacaggctgagagacagagcatgggtctgactcgttttcctccggcgaggagtgagtaacgcatggactcgtgtatcataatgggatctctgagcaaaacggctgagtctaaagcttggttgactgagttggctttttggaccatgtaagacttgaaattgaagatggatttttcttcatcgccttctttaacggcttcttgctcggttagaacagctgagattgatgaaaatggtgaagtgggtcgtttttgttttggggtttggattgaaaagggtgtttttttcaaagggtggtagagattgagattgaaagttgataaggatctggatctacctccttggttgaacatggaacaggtttgaacccatgtgagatttacagtgctcatcttcttagtcttgacaaagattgaagctttttgattttgtgaaaatgaacaaagcaaagacactctgtgctaacagatttaaaagcttgtaaaggttgaatttttaaataagaatctat

SEQ ID NO: 174 - DNA, 1059 bp, Cannabis sativa
cataattaaatacatttttttaatttcacaacaaaacgtcttaatttctatagacgaaaaagaaaggttgaaaatggctctaagtttctcccatcgaaatattttagttcctggcgagccaaattggttaactcctcgatgattctgtctgcttctttcttttcttctgtgctgctcattataaatctattggcatatccttgaatcatgcccacataaagaccaaaagttctcaacttctcaacctcttcttcatgtgcctctccaagaatggccccacatgcagcagcgcatgcatgcatcgcaccttccttcttcaaagagggataatggccccacccgacagttttggcttcatgactatcaacgtcaaatctactggctagcttctcatggtattgagcatcaatagttcctcgtgatccaaaggtgcgtgtgaactctacaatgacccgcaaaatccgttctgatttattatgggtaaggtcatcagaattggccaacaattcgaacccaaaaggtacaattgcgtccgggagaaggagctgaatatttgggttgtaagaattgtgggtcgctgcctcaggacttgttggattgggcctgcccgttaaagggaggtggtcatgagtgaagatggctgcgtggatgacgcgcaaggcggaggctgctgccatggcctggtcctggtggcccccgacaagctcacacgccgccacgcacaatgacggtgctaaattcggaggtgtggaaaagatgaaattgtacatggtctcgtgaactgagagtggtggcttgatagtgatggattgctttagatgggcatctacatcggctgttgtggaggcaaaataagaatgatgatgctgtttggacatggttattctcatgggtacgtgacataacgacttattggatttacatgtgaaatgtggaacgtaaacatgatgaacaaatcttggactgcaattaattgatagattataataaaccgccattgatgaagctctagagctcccctaaaaaaaattatataaataaagaaagtaaggaaagcaa

SEQ ID NO: 175 - DNA, 1075 bp, Cannabis sativa
catacaaaaatgggaactgaaggtggccaatatagtagagttgtgagagcaccacacaccaaaccaccattcacactaagccaaatcaagaaatccattccgccccattgcttcaaccgctctcttctccgttccttctcttatctccttcgagacctttttttcgcctctttattctactacgtagcaacctcttacttccaccttctcccacacccacttttgtacatggcttggccactttactggatctcccaaggctgcatttgtttcggtatttggatcattgctcacgagtgcggtcaccatgcttttagtgaccaccaatgggtggatgacactcttggctttatcttccactctgctcttctcgtcccatacttctcgtggaagtatagtcaccgtcgccaccattccaacactggctctattgagagcgatgaagtgattgttccaaagaggaaatcacaaatgccatggcattacaaatacctcaacaattcattagggagattcttaaggcttggtcttaccgtgattttcggttggcctttgtatgtgtgtttcaatgcattaggtagaccatatgatcgtttcgcttgtcattttgatccttactctccaatctactcaaaaagcgaaaggtttcatatactaatttcagatattggtgttttaattactatatttgtattgtaccaactcagctcggttaaagggttgagttgggttgtgatcacgtacgggatgccattactagtagtaaatagcatccttgcggtgatcacatacttaaatcacactcaccttgcattgccacattatgactcgtcggaatgggattggtttaggggtgctttgtcaacggttgaccgagatttcggagttctcaatggggtttttcataacatcacaaacactcatgtggtgcaccatttattctcaacaatgccacattacaatgcagtggaagcaaccaaagttgtgaagccaatattgggagagtattattgttttgatgacactccggtaattaaagctatgtggagagaggttaaggagtgtgtctatgttg

SEQ ID NO: 176 - DNA, 1084 bp, Cannabis sativa
agctttaattattggagtgtcatcaaaagagtagtactctcccaatatgggtttgatagctttggtggcttcatttgcatgataatgaggtattgttgcgaataaatggtgtgccacatgagcgtctgtaaggtgatggaaaaccctattgagaattgatccatagtttcgatcaactgttgacaatgctcctttcaaccaatcccactctgacgagtcataatgaggcaatgaagagtgagtatgattcaggtatgtgattatcacaatgatactattccctataaacaatggcatcccatacacgaacataatccaactcaaccctttggccgagccaagctggtataacatcaatgcagtgatgaaaatactaatatctgacatcaatatatgaagcctttcactttttgagtagagtggagagtaaggatcataatgacatgcggaacgatcatatgatttgcctgtaagattgaaacctaagtacaaaggaaaaccaaggaacaccataatagaaagctgcagggctcttcctagtggattgtccaagtagtgtttgtagagccatgctatttgagattttgggactggaacaaacagttgatcatgttcaagggagccgatgttggaatgatggcgacgatgactatacttacaagagaagtatgggaagagaagagcggagtggaggacaaagccaacggtgtcatccacccattggtggtcactgaaagcttggtggccacaatcatgaccaaggacccaaataccagcaaaaatgctgccttggaagatccaataaagtggccaagctaagtattggagcgggtgtgggagaagatggaagtaagaggttgctatgtagtagaacaaaaaggagaaaaaaaggtcttgaaggacataagagaaggaacgaagaagagaacggttgaagcaatggggtggaatggctttcttgagttggcttagtgtgaatggtggtgtggtgtgtggtgctcggtcaagtctactatttttgccaccggctcccatttttgtttcttcaaaaagattacaaagaaaaataataatgcaatattag

SEQ ID NO: 177 - DNA, 1118 bp, Cannabis sativa
cttgttcaacgtaaagacactctttggcctctcgccacatagccttaattattgaagtctcatctaaacgatagtactctcctaatattggcttcacagcttttgttgcttctattgcattgtaatgtggtattttggggaataaatggtgcaacacatgagtgtttgaaacgttatgaaaaacactattgagaactccaaaatctcgatcaacggttgacaaagcacctctcaaccaatcccattcggacgagtcatagtgtggcagtgcagggtgagtgtgttgtaaataagtggtcaaaactagcaagacacctactattagcaatggtatcccatgcacgaacaaaacccaactcaatcctttggctaagacaagttggtgtaacaccaatgtggtgatgaaaacaccaatatctgaaatcaatatttgaagcctttcattttttgagtatattggagagtaaggatcataatgacaagcgaaacggtcatatggtctacctaatatattgcaagctaagtacaaaggccaaccaatggttaaaaagaaaccaagtgttaggactctccctagtggattgttcaagtatttagagaaccatggcatttgagatttcgtctttggaacaaaggcttcatcgcggtcaatggagccagtgtttgaatggtggcgtcggtgactatacttaaacgaaaagtatgggagaagaaaaacagagtggataacgaaaccaaccatgtcatcaacccatttgtagtcactaaaggcatggtgaccacactcatgaccaatgagccctaaaccgaacaaaatgcagccttgggagacccaataaagtggccaagctaagtataagagtggttgagggaaaaggtggtagtaagaagttgctacgtagtagaacaaagaggcgaaaaaaaggtctcgaaggacataagagaaggatcggagaagagaacggttgaagcaatggggtggaatgactttcttgagttgacttagtgtgaatgatggcgttgtgtgtggtgctcgatcaagtccactgttttcaccattgactcccattttcgtttcttcaataagattaggaaaaaacaaataacactatcaattt

SEQ ID NO: 178 - DNA, 1122 bp, Cannabis sativa
ttaacattgtattatttattctctttttaatatattttaagaaatgaaaatgggaggcaatggcgaaaatagtagacttgagcgagcaccacacaccgcaccaccattcaccctaagccaactcaagaaagccattccaccccattgcttcaaccgctctcttctccactccttctcttatcttcttcaagatcttttcttcgcgtctttgttctactacgtagcaacctcttacttctacctccctcacccgctcccatacttagcttggccactttattggatcttccaaggcatcattttttgtggtgtttgggtccttggtcatgactgtggtcaccactctttcagtgaccaccaatgggtggatgatatggttggttttgtcatccactccgcgattctcttcccatatttttctttcaagtatagtcaccgtcgccatcattcaaatactggctccattgaacatgatcaaatgtttgttccaatccccaaatctcaaaataacatggctctacaaatactacttgggcaatccactagggagagtcctaaaactttctattatattgatccttggttttcctttgtacttaggttttaatctagcaggtagaccatatgatcgtttcgcttgtcattactatccttactcttcactctacttaaatagcgaaaggcttcatatattgatatcagattttggacttttaatcatcacattggtgttataccaacttggctccaccaaagggttgagttgggttgtgttcatgtatgggatgccattgttgatagggaatagcatccttatagtgatcgcatacttgaatcatactcaccttgcattgcctcactatgactcgtccaagtgggattaattgaaaggagtattgtcaacggttgaccgagattatggatcacttctcaatagggtttttcatcaccttacagatgctcatgtggcacaccatttattcgcaacaatacctcattaccatgtaaatgaagctaccaaagctatcaagcccatattgggagagtactactattttgatgacactccaataattaaagctctgtggagagaggctaaggagtgtgcttatgttgagccaa

SEQ ID NO: 179 - DNA, 4158 bp, Cannabis sativa
taattttacaataattaatgaattaattataattaattaacatgcagatccattatctgagtcaaggattagtgatttttatgtaccaagagatgaagaatttgcagaagtgaagcaaagtaattttagtttgaagactgtatactcagtaatacatgcagtgattcccatactcagacaagtcttaattgatgaaaatttcccatacttcactgccattgatgttctctacgatgaaggcattaaaattccttctaatgctgaaaagaccttaattcaaaccatcaaaaatgtcaatgcaagaatatacaaaactgtttctgatgctgatgattttttacagtttcagcagcctccaaccatggacagtaagcactattataaccttcagtagtatttaataatttcggtttatatcgtttttataatgtaaaattatttttattaaccatcaaattccgttaccaaacaaggtaacaacttcttttgggtttatagaggtatttttagtattttcggttcgtaagttctaactttcataaaattttcataaaataattatgaattgtcacaaattttcataaaattaattatgagcaagttaggatattaaaatcaagattaaaataatatgtcaataagctagttgaattttaattttagtatattcttaaattatccgaaatttttaagttcttatcattataaatagtccacttacaaaagaaaaaaaatatattattacaatttcttgtgtttttttcctatgaatttgttataatttttattttattttttcccttttggttatgtatatatatatatatatatttagaggacaaattcttctggtttagagatgaagagttttgtagacaaactattgccggtctcaacccttgctgcattgaattggttaaggtatatatatgctttttcatatacatatatttttatgagcatattgctagtatttttacatgtgttattgcaattaagttaacaatactttttaagagttattatattaaattgaatattgttattaagtaccaatagtgtttactactttttataagtagcatcttacgatttattagcgatattttctaaaatttattttattaacttattatatgaaactcaatacttaattaatataccagcgaggaggatacaaggcaccaataatattatatgagactcaatacttagttacactaattataatattaacatgtaacatcactgattcttttttgaaatttttttaggggataaatatagaatattttaagttgttcttattggtatatatgttatccaaaatccatgcaggagtggcctttgaaaagtgaacttgaccccacaatctatggcccaccagagtcaaaaatcaccacagaattggttgagaaattcatcaaagtatatggctacaataatattaatgaggtacatatatatatacaaattcaatcacattaattaattaaaaacaatcatgtagtccaaaaaattaatgcaataatacaaaatggaatttttatactaatgttatgtttattttatttatgcaggctttaaaagaaaaaaaattgttcatgttggattaccatgatgtattattaccatatgttagcaaagtaagggaactggaaaataaaaccttgtatggatcaagaacacttttcttcttgactccttatggtacattgttgcctttggccattgaattgactcggccaccgatggatggtaagccgcaatggaaggaagcctacaccccgatgaattggcattctaccgatctttggctttggagactcgcaaaagctcatgtccttgctcatgattccggtgttcatcaactcgttagtcactggtatttaccattactaataaaattatattttatttttactaataattaaaaatacttgtaagtaaaaagtatttagttacaaattatcactaacatataattttaatatttcagcaaaaaagaacatatagttttagtagcaggatcaaccctgagatttattggaccttaagtatgaacaaaattatttgggcccttataaaaaatgtggtattttaaaaactactaaaaaaatataggtatttttaaaatattttcttttggtctttggggctttgaactgggtggaccctaagtgtaggtaggcccgactctaggcataagtgggctaggcttgtgcttagggcccacctagctcaggggcccaaaaaaattctccttttaaaattacactttttttttactaattataaaaaatgccacatcttttttctaaataatgacccaaaaatattttctttactataacccactgaattttagggccagccttaagctaagcctaacctttcttaacccttagaagtcacaatatttgtgtgctataaattttagttaaaaaaactttgacaaataaataaatatatatttttttactacaagtgatatgttatgtattaactataactttttctattttaacgtggaaaattggtgcatgcatgcaggctaagaacacattgtgcagttgagccatatataattgcaacaaatagacaattgagtgcaatgcatcctatccatagattattgaagccacattttagatacacaatggagattaatgctcttgctcgagaaagtttgatcaatgcaggtggtatcatcgaaacagcatttgcacccggaaaatattctatggagttaagctccgtcatgtacgacaaacaatggagattcgatctacaagcattgccagctgacctaattcataggtatacatttgtttaattataacatataaaacacgaagataattatatttcgaacattgctatttagtattttatggtatctaataaagatttctaatataaaataagatatctgggacctggatatatatgtcacatcatcatgttatgtagttataacatagatagactttttgtaaataaatatcatttttagtcacaacaaaaataagtacttattactaagaaagtagtatttagattgttactcatttaattttaatcacagcaaatgttgtggttaaaaacacatttagtaacaataaattatgattttcgtgattaatatttttagttatggactttttattagtaataatataggaatgatgatttatgtttagttacaattattttacttttaagtacaaattttattgcaactaaaagtaagattattttgtagtggatgttgaaacttaattctatactatatacttttcatttatatgatctcatcataattagcttgaaattcatgtaggggaatggctgttgaggacaaggatagtgaacatggtgtaagagtaataattgaagattacccatacgccaacgacggtcttctcatatggagctccatcaaacaatgggttactgactacgtcaaccactactaccctatctcaagtgaggtagagcgtgatgaagaattacaagcatggtggacagagatcagaactgtaggtcacgctgacaagaaagacgcacctgggtggcctgacttaaaaacgaaacaagatctcatagacattgtcacaaacatggcatggacagcgtcagctcaccatgcagctgtcaactttggacaatatgcttacgctggctatttccctaaccgaccaaccataacaagaactgttatgccgtcagaagagaaggagtataacctagatgcgtggaaacacttcaaaaatagtcctgaagatgcccttttgaagtgcttacctacgcaattacaagcaggcctagttgtggccgtgttagacgtgttgtctactcactcgccagacgaagagtatcttggagacaagatggaaccctcgtggggctcgaatcttattatagcggaagcttttaatcggttcaataagaggatgaacgagattgaaagtatcattaatgaaaagaatgataatgagaatttaaggaatagacatggagctggaattttgtcttatgaacttctcaagcccttttctgagcctggtgtcactaacaagggtattccatatagtatatctatttgaaataaattttaaacattccatatagcatatctatttgaaataaatttta

SEQ ID NO: 180 - DNA, 7199 bp, Cannabis sativa
catacatgtgagtcagtcacagtactcaaacaaaagcatcttaaaaccatgtcttttatgatgagcatgaatccttctccctcctcgccaccgccaccgttatcgtcgccgtcggaatcttcctcaacgccgtcaacactgccagtccgtacgatcccgggaagctacggatggccgttactggggcccatctcggaccggttagactacttctggttccaaggcccagatacgtttttcagaaaaagagtggagaaatacaagagcaccgtgttccgtaccaacatacccccgacctttcctttcttcagcgttaatccgaacattgtggccgtgctggactgtaaatcattttctcatcttttcgacatggaaattgtcgagaaaaagaatgttcttgttggagatttcatgcccagtgtcaattacactggggatattagggttggagcttatctcgacacttctgaaccacaacacgctaaggtaattaatttcctctcaaaagttatatttaacaaattaattaaaatctttgattgcttattctttattgtgttaattttggattatttgaactgggtttttatatattattaagatagtctttaataatttcttgcaacatataattttaagaagacagaaagagagagtgagatgtatataataatgtctagaaaaaaaaaaaggactaggattatataataagatatttagatatttaaaaaacaaatctgagattgatttttatttattattttgaaaggagcaaatctgagattgattgaataattgggatcagagccaatggagtttcttaacactaagttatgattcacattttttttattatattattatttttttgggatagggatttacatgtttattaaattaattgtggtgtttttttttttttttttcgaaatcaattaattgtggtgttagttcagagttctcacttctcttacatattaataaatggcttaatataagtcaaaataaataaatggcttattaaattttacataagaccgaggttagattttttttttctttgaatataaaggaaaagactaaaaggcccaaatgtgtaacacgattggtgtactattccaatcaaatgaacaagtggacgatcttggtcacacgtacgggcaaatgcaaatgcaatcaaatagctccattcgaaatttcgtatgtatgagattctgaggggagaattacttttgccacattattttgggaccattctcttatactaaacaccaattgtccttttcctatcatatggctctcctaagaaccaatgaaattatgtgacttttttggtttttttttttttttgaaacaatgacttttttggtttaataaagagtaaatactatttggacattatgttttgtaaaagttattagatgaaccatctgttttgttaaatgataaaatagaccctatattttctaaaatggtaaaaataggaccttcagctcaattttcaacaattttatttttcaatacaactaactttaagaaaatttctaacacgaacagattcagaaaatataatcagttttgtcataacatctatagatcagattatgattaagttttattttgataaaaaaaattagtgcatggtcctatttgtacaattttagaaaatataggatttattttgtcatttaacaaaataatttgttcaattggtcaattttgcaaaacacaggatccaaaatagtatttaccctttaataaatatatatcctagaactaagactagcagcacataaatataggtatctagcattgttttagaaaataatattacgaaaacatagaggcttttaagttgtattttcagttaaaagtaagaatagaaaaaaaaaattgttattttaaaaaaattaatggtgtttgactcaagttttttaaaactatttttaattttttttttctaaaaaaaaaaatcaataccttacaccatactatgacatgaactcatcctgcctcaaaaacaaaattataaataaagagtcgtttgaaaacctctacataaaaatggggttttgaaatctcttgatctctgatatacttctcaggatatttgagtcgttgattcggtcatttgaatacttgtcctctaccgtttcgagttttctctttggcttcgttctctccgttctcgccttgcatctgtattctcttcgccttcttcgtgccctgtcaggtctggtcccctattcgacgatgccccagtagagagatgccttgcagatgtgaacttttgaggctcccttcagatgacagtggagagtttggtgcccgtcgaagctctgtttgtggccttagttttgagcggtcttgaaggtattagtttctaacgggtgttggctggagcattaggacatgcctctcatttcctttggggttgggtttcttgttttagtgagagcttttcttcctcaaagcctattgcagtcagggtctcttggtatgtgatttcttgatttctttttgctttttgtttagattatttagtgttgttgctttagggtttctatttttagggtttgaggttgtgatgtttgagtgggatctctgttcatctggttgtgggttttggtttaagatttacatttttctcgacaattgagaggtttttgtgattttagattgggttgttgtggacttttttggcctgagagtttctgtgattttactttttgtcactcccctggttgttgagctgtttattgcatattctgggagggagtaacatttggttagaagtttggttccataacattaatgataattgggtttttgctacctttgtggaggttttggttcaaggtggggttgtcgaacatcacccactggtttcatgaaaggggttgcttgcaattataagaactctaattcgaggttctaaaagaatgtggttaaggctaaggatgttctaaagaagggtgcttgctatgtgatagctgatgggaaagatatcaacatttgggaggatctgtgggtggcccatggtatcgatttatgccccattgcaaatggggtttcagcccccagctttatgactgtggcaaatttattactagaaaatggggagtaggatctttgtaaactaaataggttgtatgatcatgacacaatgagtgccactttggtaagggaaaggccactaggacatgggagggatcgctggatttgaattaaagaattgaatagaaaattctccaccaaatctgcttaccttttgcaggctcttgagagagaccaacattgtcaggtcgctccttctctgtggaataaattttggagcaacaaaacacttgaacgacataaagtgctttggtggagcattctatccaatgcacttccaattcgatcccttcttatagatccaatgaacattaaagactcctcttgcccgatttgtgggagagaagaagaaactatggaacatctttttcttcattgtaacatggcttctcatctctagagagtcgctccttggggtatcattcctgttcaggaaccagaaatcaggatatgagactatgtcaaattcatttggaggttgctagtgagagatgtgaagattgatgatgtatttttatatgtgtctatactagttgacactatttggagagcgataaaggtacataataaccatctaaataacataaatttttatagtgactctattgctcattgttatacaaagtattgccaatctcttttggatcaatcttaactttcccatgctatcactgagtgggctcctctgcttgcggactggatcaaacttaactgtgatgtgggattagggaggggcctatgatgcttgcggttgtggctcaagatcatgttgggaagatactttaggtcggtactaagcgcctgccttttgccaatacttttattgggaaaattatttcttgtctgctggcattggaacttgctgcagagaaaggtttagaatttgttattgtgaaaagtaattctaacacagtcattaaagcaggcaatggtttgaacccgaggtagaaaattgatagctatttgactcattgtagaagccttatgagctccttcttaagatgtgcttttttaaaggtttctagatcttttaacttaatttgaagcccacaatttggcgtaagtgggcgtttagtaaaaatgtaactggtagttttgaaacatcctcaataccatataatattgtttgtaatggccattgggcctaatttgatttcatttattaattacactttttttgaaaaaaaaatgaacccatcctaccttgggccgaaccttgacatgaacttggacttcgtcccaaatccaaacccggacccagactccagacctggggctaggtctgaggtcctaagctaggtttgtggtctgggtctggaacctgtccggggtacggggtcttaggtctggggttcgaggtctgttgtttggggtccaggttcggcgtttgggatctaggtttatgttcggtctagtgtccggtgtctggggtctggggtccaagtttaggtctagattcgggttcggggctaggatttgggtcggggtccatgtttgggttcagggtctgggtttgtataataaccaacaaaactacttaaattgcaactaaaattcacacaacaacccatataaactatcggagcaacatcaattgtaaatttttaatatatatatatatatatatatatatatagaataattctccaaaaaattatcttaaactttaattatccctcttggattgtggattaaaatatcccaagttgttttttttttcttaccattaattagatttaaaatatatatgtgcattacatatattttaataattaaaatttttaaaaataatgaaattttaatctataaaataataaagtagttcaattttaaaagtttattaatatattttcttttttatttgttatgtttattaatatgaattttaaattatagaataataaattatatattataattttgtacatattatttaatttctagagctattaaatttattgaaaattatttattttatttatttttatttttttattttattttattacatatttatttcacattattctttagtgacaaaaaaaattaaaaaaaataataaatttattaaaaaaaaaaaactgtatagtcctgttctaagccaaacataatacaaatatattttcttttttctcaaagtaaacatgatataatacttaaattatccaatatccagtccaatcatgcattactttatataccaaaagagccctaaggtattcactacaagcaacatgtttttgaggggccatgatatatgaaagcataataagcatataattttgcaggtacattaagtttagtggttcattgaaatatcaatctaaactttagaattagatggcagagtgttttggttgaattgagtcatattataatgtcttaataatgctattttatatagagatgcttataaatgaagtggttgaaatcaatgaagtacaggttaagaacttcgcaatggatgtattaaaacaaagctcgaagatatgggtgggagaactgacatcaaatctgtcgacgatgtgggacacaatagaaaaagacgtatctgagaaatcatcctcatcctacttagccccacttcaaaagttcttgttcaacttcctggtcaagtgtctaattggtgctgacccttccaactcccccaagattgcagagtctggctacatcatgctcgaccgatggttagccttccagctccttcccactatcaagattgggatccttcagcctcttgaggagcttttcattcattcttttgcctatccttttttcttggtcagtggtgactacaataacctctccagttttgtagaggaatatggttagttaattatactaattactatctctactctctacacaaatatttatttagtgctgcattgcattgttttttttagtgtctaacaccccaccttatgttgaacaatcatggtcatataaagctgcattaattggtattaggcactattaatttagagatatgtaggaagtgatatattgttattagtacaccattctctccaaaccatacatacaagtgcacgacgagcccatgcaaagtgtttggttaagatatgccagattccaccaaatatacaaatggaacctaatcatacatgtcctctaactatatcttccaaatcattcacactaacaattcacctctctccaccaaaagaagatttgagtaaataaccaaatatggtggtgcggtgatagtgttgtgttagactcatgaccccattaatgcattggcaaaatgattttttttgtaaaatttattttgcatagacaccccttgtcattttgcaaataatttccacttgggagtaataatacactaaaaattaatagtttgagaaattatttgtggttaccttttacaggtaaagaaatagtagcgagaggtgaaaccgagttcgggctgagtaaacaagaagcgattcacaaccttctcttcattttgggtttcaacgccttcgggggattctctatatttctaccgagcctactgggcaccgtggcgagtgacacaaccgatctacaacaaagactggtcaaagaagtcagacaaaatggcgggtcaactctgacgtttgactcgatcaaagaaatgccactcgttcaatcggtcgtgtacgagactctccggctcaatccacctgttccgctccaattcgccagggccaggaaggacttccggctcagctcgcacgacgcggccttcgaggtgaagaaaggcgagctcctatgcgggtttcaaagccttgttatgagggacccaaaaatattctcggaaccggagtcgttcatcggggaccggttcatgaaagataaaggtctgttagattatctttactggtccaatggacctcaaaccggtgtgcccagcgtcaccaataagcaatgcgcgggaaaagatatcgtcacgcttacggcttgtttgatcttggcttacaccttccgtcgttatgactccatcagcgggagctcaagttcaatcacagcccttaaaaaggcttaaactactgaatcagaggagtatggtgatggtgatggtaatgacaaactta

SEQ ID NO: 181 - DNA, 6687 bp, Cannabis sativa
atgaacaccaaaatcattaatggttgtttttcttttattatgattcagggagaaagtaaagtccaccaatatgggcgcacttttggagaagcgaggaaaagagtttttgggagtcaagtataaggatcccatttcaagcttttctcatttccaagaattttctgtaagaaaccctgaggtaataataataactatttattattttatttgagggtggtttttatgttaattatttgtaattaatggttaatattatataatgttaggtgtattggagaacattactaatggatgagatgaagataagtttttcaaaggatccagaatgtatattgcgtagagatgatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggtatatatacattgaatttactttaaatctttctgtttttacatataacaagaattcataaagttaatattatatggtttatttggatattgaaactatgtatggtaattaatttgaaggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgactatcaaaagcaaaagccatttttacacaggtatatacatataatatatggagaacgttctaatagttacattaaatatgtaaccatgatggttacatttatttttagttgttggattattattaaacgggtatgattaatttaaaaaattactaaaaataactaataaattacctgcaagatactaacctgatttgatttatttcttttaaataaaaaaattataaatattaatttctaaaaatgttttattaaatactttaattaaaaaataatttatttaagtctaaaaatagaaatttacctatcagtgaactgttataacaggttataaaaaaaatgtaactatcaatactcacgcataaatatatatatatatatatttactataaatactcattgcttatatagacatatatactaacaaaaaattacgtgcgaggcacgtatactaaattttatgttagtttttttctacgttatttaaaagtgataccattaaaaattaaaggaaaaatattattagttattaggtgagattattattatgtgtatctaaataatataatttataattttgacaattaaaagtaaatatcggatacaatatattagtatttaagaaagcttgataatttaaatatgttcttaagttaatccaaaaataaactaaaaattgatgttacaatacttaaggaaacataacttaaatgtgtgtaagataaaaaagaaaattaaaaatcaatctctaaattgtagcaagatctttttatgtttacttttctttgtaaatttaaggtgtaagctgaatatcactgttaaaatgccaactgaataattattgtattgacttcgattttactttcacgtatacacatgtgtaaacggtgttcatccaactgatatttaatggtgatatttaaccgacgcatcaaatttacaaagaaaagtaaatttaaaacataaaggaattttgctacaaaaaaatataaggggttaagtgtaaaaaaattaaaacataaagaattttactgacaattacctcatatgtatatatatatggatattttattttagatttattaatttaaaaaattatatctatgtctatgttatattaaatgtagatacatgacaaaagtgttattttttttttctaaaaaaaattacgattttaaatttaaactcaatatacatatctctcttatttttgaccttatattaaatgtgtctatataagatattttttttttgatattgcaatataaattaaacacattgcatatatttttttttttttaaaaaaaaaattattattatttttttttaccaaaaaaatatattttattattattatttaaaaaaattaaaaaaagtgtataaaatttcaaaaacaatgaaaagtattataacatcaaaagtattacactaattaaattaaaattataattaataataaataacttgtaaattaaatatttgccccccacttgcaaattctctcccggccactgacgataaagtatacttctcctaattttatgccaatagctagtaatttagttttaatttgctttttccctttgccaagatagctagtaatgttgttgtgttatcaatttctacaatatataatgtcatttctctttatattatttagtaagaaagtaatatatatacatatatatatcctattttagcttttttttttacttaattaatttccttcgttttcaggatcatattattcgtgggaagaagcgtattcccttatacaggtcacattctctttatccgattttaaatttttaattacatttatcaattagttaaataaaaaatgacatttaaatatcattaattagtacacattcaaaaatttatcattcctgacaaataataataaaattattattattattattcattcatccatctcacaaaataagttaaattgtgcagtagagttgtggaagccaagtctcccatggccattgttattccttgtagtggctctaatattggtgcagaattgcgtgatggcgatatttcttgggattactttctagaaagagcaaaagagtttaagtaagttaatttattttacttctttttgttacatcaatttcaattgtttaattcgtagaaattaatgtggaatatataaacagtagaggtgaatatacatacatatctatacatatatgaacatttggatccattggagtttgaactaaaacaaatgtgaatggtagtggaaaataagtatcacgtttcttattaatcaaggggttttaatcaagttgggcattattaatttaggagttattttggggcttaactaaattcatcttatttttttatcttattaatttattttttttaattaaaagtaacacaggcaaattagttctatgctgacaaaaagaattatatgattatttaagtattatatctagaatattattaaaaaaaagaaaaaaaaagtattatctccaaaataataagcatatatgtgggactaatatcttcttgagtggtacctgcagaaattgtgaatttactgctagagaacaaccagttgatgcctatacaaacatcctcttctcatctggaacaacaggtaattgaagccaccgccatgttttgaatttattaaaagagcatttggaagtaactgatattacatatttgtatgtagacttaatgatcaacaatttaggcattatatatcctctgtttaagacaataagagagtctatactgataaaattaaagaaaaattacagttttacaaatattgggtaccgctaaaaataaacattgaatttatgtcgcttacaaacgtaaaactttgagtacttatgccgctattcatccttaaacaaaacatgtctacaactaaacttttaagagcgcaaactaaaaggtaattcagtaaataatgatgcttggtatgaaaaagaacacattctcaaaatcaaagaccgttaaaaaatctcacgagcccccttaatatccttaattagttttgttcatcaatctaaacacttttgatggaagacaatattgttttggtataggggagccaaaggcaattccatggacccaagcaactcctttaaaagcagctgcagatgggtggagccatttggacattaggaaaggtgatgtcattgtttggcccactaatcttggttggatgatgggtccttggctggtctatgcttcactccttaatggggcttctattgccttgtataatggatcaccactagtttctggcttcgccaaatttgtgcaggtaaactaaacttccattaactaataaactttttagttatgttatttatgtgttttgtgcttttctatatataagttatatttgtaaatgtctaattcatcatagaagggtgaaaacagaaagtacaccaataatcacatcatttttattattattattttttaaaataaaaaacatgataagtttggtatttagctaaaacattttgactacaattttattttccctttattttctctttattttaatgtttttataatatgtgtgtaggatgctaaagtaacaatgctaggtgtggtccctagtattgttcgatcatggaaaagtaccaattgtgttagtggctatgattggtccaccatccggtaaagaattataatatattttccaataattcttatgtatgcacataacatcagtctagattatattaaacgtattatatggatagttaaaataagttttatatcccttcttgtagttgcttttcctcttctggtgaagcatctaatgtagatgaatacctatggttgatggggagagcaaactacaagcctgttatcgaaatgtgtggtggcacagaaattggtggtgcattttctgctggctctttcttacaggctcaatcattatcttcatttagttcacaatgtatgggttgcactttatacatacttgacaacaatggttatccaatggtaattaataaacttataaaatataatataaaaatattggataagacttttcaaataagatttgatggatcaatttaaattcacaattcttgtattaatcccatataaaattatgttacatttgacttgtttgttgtattatatatatactttatgacatgcagcctaaaaacaaaccaggaattggtgaattagcgcttggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaaggtatgaaatgaattatgtgtgctattaattagttaggttgccctgcccaccaataatttattttttatttattttaaattaactattttgatcatatatttttttttttataattcttaattaggttttagataatcacacatattcttagttcatagacaaagtaggaaattatcctggttaagaacctaagttcgatgtaaacttagtcctacttcagcttggaatacgatctcagatttcgttattaattagcaacaaatcaagtacatcacagttgagtaaggattctcaatcttgggttcaaacaagtttttcactttcacactctcatccgagcatacaaaacgtcaatgtagctcattacaaaaaccgaatccccaaactctttcacagtactctctccttatctgatcttcttgcactctctttctctagattcgtgcttctatcgtgatctaaaaaatattcactaaataatgaataagacttgtcttatataggcaacttagtaagtagctaaattaagaaaggtcagctaaatcgatcttggataataacaaatgtagctaaatctgctatatctcttgttacttttggtaatttgataggattgtgactctacatatatatatttccaactatagatttgaattgaatacgattttgtttatgtaaacagattagttccatagagattgaacgagtttgtaatgaagttgatgacagagtttttgagacaactgctattggggtgccacctttgggcggtggacctgagcaattagtagttttctttgtattaaaagattcaaatgatacaacaattgacttaaatcaattgaggttatctttcaacttgggtttacagaagaaactaaatcctctgttcaaggtaccaatttcatcattattaacttttttgtttgttaatttttagtcttgactctcaagggtgtatatttagtaaaaaattagactattcttatcctatacacttccacactatgatgatgatcttataattgtatactaattctaatttggtgtttggttaattattaaataggtcactcgtgttgtgcctctttcatcacttccgagaacagcaaccaacaagatcatgagaagggttttgcgccaacaattttctcactttgaatgaagtgttataatataatacatacatatatgctttgaataaagtgtagttgattacatagagcagtttgcattatcaaatgcactaataataagttaaatgctctaaaatatgtgttaaatcttatggcaggattatagatttgattaagatagtcatattgaaccataaattttcattcaacacatttttagtgcatttgacattatttttattgaatttaataaatactgactacactgtatagtccactacatcatatcccttcaatataatatttgtgatttagaataagaatagttacttttaga

SEQ ID NO: 182 - DNA, 1416 bp, Cannabis sativa
atcacacatacacaactcattattatcataataataataataataatatgaatcatcttcgtgctgagggtccggcctccgttctcgccattggcaccgccaatccggagaacattttaatacaagatgagtttcccgactactactttcgggtcaccaaaagtgaacacatgactcaactcaaagaaaagtttcgaaaaatatgtatgtattatgaatatatatatttccaactaataatacttttccatgtacgtatacgcattatctatttatataataaaggaatttttacatcatatattaagcatgaagaatttatgtttaatatcttgtatcattactttctttatatgtatatgtaggtgacaaaagtatgataaggaaacgtaactgtttcttaaatgaagaacacctaaagcaaaacccaagattggtggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactcagtccctcagtgaagcgtgtgatgatgtatcaactaggctgttatggtggtggaacagttctacgcattgccaaggacatagcagagaataacaaaggcgcacgagttctcgccgtgtgttgtgacataatggcttgcttgtttcgtgggccttcagattctgacctcgaattactagtgggacaagctatctttggtgatggggctgctgctgtcattgttggagccgaacccgatgagtcagttggcgaaaggccgatatttgagttagtgtcaactgggcagacaatcttaccaaactcggaaggaactattgggggacatataagggaagcaggactgatatttgatttacataaagatgtgcctatgttgatctctaataatattgagaaatgtttgattgaggcatttactcctattgggattagtgattggaactccatattttggattacacacccaggtgggaaagctattttggacaaagtggaggagaagttggatctgaagaaggagaagtttgtggattcacgtcatgtgctgagtgagcatgggaatatgtctagctcaactgtcttgtttgttatggatgagttgaggaagaggtcgttggaggaagggaagtctaccactggagatggatttgagtggggtgttctttttgggtttggaccaggtttgactgtcgaaagagtggtcgtgcgtagtgttcccatcaaatattaataaaagataataataaatgataaaaagaacaataaagaataatgcccaat

SEQ ID NO: 183 - DNA, 689 bp, Cannabis sativa
tcgactgtatgtagcataatgtgatttatataattatcaaaaaaaaaaaaaaaagaagaagaagaagaagttgagaaagagaatggcagtgaagcatttgattatattgaagttcaaagacgaaatcacagaagcccaaaaggaagaatttttcaagacgtatgtgaatcttgtgaatatcatcccagccatgaaagatgtatactggtaatttgtgattattttgtttcataattcatatacatttaactaattattataataatagtatagtgatgaataattctaataatgttgtttgcattgcaggggtaaagatgtgactcaaaagaataaggaagaagggtacactcacataattgaagtaatgtttgagagtgtggagactattcaggactacattattcatcctgcccatgttggatttggagatgtctatcgttgtttctgggaaaaacttctcatttttgactacacaccacgaaagtagactatatatatatatagtagccgaccaagctgccttcatcttcatcttctcaaataatatatctaatatctaattatataataataactacttaataaaagactgtgtttataacattaaataataataataataaagtcttttgtagctcagctcatatctcttataattttttattttttattttttttttgattga

SEQ ID NO: 184 - DNA, 496 bp, Cannabis sativa
tcgacagtatgtagcataatgtgatttatataattatcaaaaaaaaaaaaataagaagaagaagaaagttgagaaagagaatggcagtgaagcatttgattgtattgaagttcaaagatgaaatcacagaagcccaaaaggaagaatttttcaagacgtatgtgaatcttgtgaatatcatcccagccatgaaagatgtatactggtaatttgtgattattttgtttcataattcatacatttattaattataataatagtatagtgatgaatataattctaataatgttgtttgcattgcaggggtaaagatgtgactcaaaagaataaggaagaagggtacactcacatagttgaggtaacatttgagagtgtggagactattcaggactacattattcatcctgcccatgttggatttggagatgtctatcgttctttctgggaaaaacttctcatttttgactacacaccacgaaagtagactatatatata

SEQ ID NO: 185 - DNA, 7381 bp, Cannabis sativa
ataattaagaagaaaaaaaaacatacaaatatatagaagtaagagacacacacataccctaatggaattttttgcgattgataatgattctgagcatttgttttgtagatgaaaactcttggtggagcaatgtttagagggaaaattattgtaagagtatttaattggtgttttggggtgtcgataacataataatgaggttttgggattattattgtgaggatttaataaagtatggtaattagtttgaaatgaaaaggtacaaactgatgagagtcccatgaagattattattgattgaatattaagaaaaaatgagggaaagaaagggttagaataacatagtgatatcaataatgaaaaaataacaagatttatagtaaaataatttttgcttggtttcaaaatttaaagtcatccatgttttagaagtcatgcatggtaggcacatctatctttcttgcctaattaactaattatggctatcacaaaccatgcatgcatgtagagctctacactacgatgagattaaatttatacaatcatatcaaaattaatattttgatttagtgttgaaaatttatgaattattgtaactattaaaacatgttcggaaatccctcatctcgaccaccaaataccatctagaatcttcctacaggaccctcctacccggagcctcacatctagaacttccaatccaggacctcccaactagagccttccaaaataaatccttaaccagacacacacacatttttgaaactatcaatttggaccccatctaaaaacttttgatccagaatataccatccaaacctcactcatcacctattcgggatcccttgataaggatactaacaaccaggtcacaccaatccaagcaaccaaaactctaggctcttaactctagattcaccaaccccaaacctaatcattgaaaacactgtctaggactacccttccgaaaccttaccactcaaatcacatatgacaagtacaactcattcgaaaccattttgggcagaaaagagttcaaaactactcaactttcagaccttcaaaattaagtcattccttgaccaaaggctatcaaacttaccaaaacaagagggtaatctaaacaaaacatacacttcaaaagcatctaaaaccacctaaaactaatttcaccatattgttgagtggactttgagttcaaaaactcgaactcctccaacacgaactacatcagctaaaatcttacaaaccaacacagaattcaaccttaaatcaaaccaacaacaattttaagctcaaacaaactctacaaaacctaacccataaaatccaaactaattacaacaaaagcttttcttttaaaaatttaaagagacaaaggagataggttaccttgatactccttctaattcaacactaactctagttagaatttcttagttaattggtgtgtaaattattataaaaaatctggtttgaggttgttatgacctagaaatggaccaaatggatgtgaaaataacatttttacatgggagatcgacttgaagaataaatctaaatggaagagcttgatggatacaaaagcagcaaaccaaaccaggtatgcctcctacaaaaatcactttatgggcttaaacaagccccaaggcaatggaatctaaggtttcatgaatttttgatcagtattgggtatagtagaagtgattatgacccctgtatgtattataacaatgaaccaatttggttactattgtatgttgatgatattctcataattgttaacaaaaggcaggctatagaaaaattaaaacaaagtctgaatacagaattcgaaatgaaagatcttggagaagcctagatgatacttaggattgacataagaagagacagatcagagaagctttcactatcatagagaagttacatacaaaaattgatacaaattctgcatgagtgaagcaaaaccagttagcactccctggcccacatatttcaagttaacacagagccaatcacccaaaattgaagaggagaaaatgtacatggatacaaactcatgtttgtgtatgaggtacatgatcaagcattgatcacatcagttcaagtaataaattcatgtttgtgaatgatatatatgttgtgaaaggaggatacaaactccttgttgtagacttccttacaaagcatcttgtggtgttaaaacctggcagattcttctcattttgctttcactatcgcctttttaggcatgaggattcgctgtggaatcttcatgtcaactctcgtccacctataactgtttacaagtatggtttttcttcattttaagtgagagcgaacattaatttaaatataactatgtaaagatttgctactaaattatatcaataacaataaattttatttataatatgttgagagtaagtgtgttttataataacaaggctctaaatgaaaacatatactaaatattcagcataatggagcttccacatgaactcgtagaattttctgcctgcttcctgcattgtacaaccaatatatatatataaataattaatgtatgattaataattaattagaaaattatattattttttatgtatataataataataataataataataataataataataataatataataataataataataagtgtatataattaccgggttgtaatttgttagagcaaactctcgagtctgtacgcaattaaacaattaaaaatatttataaatatgtgaaaaaaattattataataatatatttgctcttaatttcttttgtgagaatttaaatggcaaacctgaaaaattaaccaaaatgctaaggttgcatgtgaaagtaacattacgttactcttgaaagcctgtaattaaataagaaatgatgaaaagacaattaacattaataaataaaatacttgaattattttagaaataataataaattaatcaagaattgaatggatgaatcttatgagtattatttcgggtgtgactcatatggtaaatataatatgatatgaatttaaattttatctaattaaattagaatatgtattaaaatgtaatattaaattatacaaataataattaatggcatgtgtgtcagacaatattttagtacctcataatgtataattaataaaatacctgtggccaaataatggcagcaagtatagcagatacataggttagaagaacaattccagaacaaagaaatgttatgtttttagcaccatatctggttgccaaggttgatatgccatgcctatatagaaataagataataagtaatattattaagtgtgtgtttaaaaaaaaaaaaaggaaaaaaaatgataataaaatatttataaatgataattttaaataaaataactaattaaactaattaaggttggcataagaatttaacatacttggagtcgccttcaatgtctgaaacatctttacataaaaataaagctgatcccattgatttaacaaaggctaggatgaaagtaaaaggaggactgaaactcaaattcaaattcaaattcaaacacatgatgagatagttataatacatcaaattttgataagaatcgttcagcaagaaaaaaaaaaaaaacacaaaagtcgaattatcccaatgaagaaataattaaaaatcattttacatgatatacctcattaatttaaattggcaacatttttttaattaactttttatttttttagaaaaatgaaaatactttttaaaaacatcttttataaaattagttttcactttcatttttaattgagaattaaacttttaaaaatttgaaaacaaaacaaaaaaaataatcactttaatccattttttaaatggttttttttcttaatcagtattttggtcgactttgaccccaacttagaccttggtacccaaaattcggccctagccccagtcgtggactcggaccacaacctcgacctcagacacatacttgaattcgacttaaataaaattaaaattaaaataaattaaaaaaaataacagtggttatagagaagtacatttctattttttaaaaaaaaaaaattaaaaacacaaattttacttttatttttatgattaaaaattaaaaaaaaaataaaaagtattaccaaacgttcccttaattgctaaaatttaaagtgatcattggaataattttagttagtctagaatttgattaatttctcattttccatgatttttatttttttaaacaaaagtacatgattcttcttttattttaaccttttttatttatttcaaaacaaaatctttcatatattctcaatatttaattaatacactatgatgacttttttttttctttcaaaaattatgagaaaagcaataaaaattagacttaacaaattaattacatcgatagttgtctttgaatatttttggagtacaaatattgtaatcaaaatgatggtagtatttatttttctattttctttagtttttttttttttattgtttaattactaacaaatatattgatatgtgtaatttttattggtattaatttgtggtagtacccacctcatctcaaatggtaggccaaaagctgctctgctagcataataacatgtgaaatttacgatcaccagaccctgcacagtgttatatattttaataaataattacctcatattaaattgatttcttttgaaaaaattaattaattaattagttatagaaaaattatcaaaagttacagttaaagataagaagtagtaaacgtgcactaaaatactacttttaataacgtagcagtgttgctataatattttgtgtaacattcattaattactcatagtaaagtaaacagtaatgatatatatataaattataaaatatgctcaggccttaatatgaaagtgatatttttttctatatatattatttgtgtgcatatatcattacttataataaaataaattaccataaaataactggaaaatgcggtattaggattttgcttccatctaaagggtggaacagaatagagagctccaaccaaaatactaacacaatacagagaaataaagagtggtccacagttcaattttatagttagtatcaacccagttagggccacaattatgctcataatccaagctgtttcaattgacatttcccctgaagctagtggtagatcaggcttgtttatcctgataatgaaaattatattaattaagtgtaatcaatattagtttaattttacaaaattaaaaatgtagtgataattgtacctgtcgatgtccaaatcggtaatctgattaatgatattagtacacaaattaacgctcaaaataaccatcaaagaagagaatgccttcaacatcagagaccaactcaataaatttgtgttatgaaacaactctctcccaaacaaaccactgttctccaataacaaaacaaaattaatatacatcaattgcaatcaagttggtgcacatatatacacttgatacacatattacaaaaaatcttattttaattactacaaaatttataactaaaataaaaatatggtggactaaatatgttagcattttattatgaataagagtgatgtttagtttcataaaatctaaattgtgactaaaagtcatcattaattataaaggatataggttttttttcttagtgacacatgattcatgaataaaggaaattttcgctcaaattataaaggagtttttacatttcatattgaaatttttaatttttatacttttttattgtagggtaaatttttttcaaaaatactgtttttttttttcgaaaatactgtgtaagttttatactgtgtagtgtgtactgtgttaagtttttactgttgtttcacggttaattttagtttttcactgttgttttaattattctgttttgttgttttgcgattgttcaataaaaagacattatttttataaaagaatttcatgttataataaaattataaatttttacccaaaatttaatatttttctaaaaacttctaatttttgttagaaatgtgtcccttttttcctattgataagagtgtattcttgaatgtgatcaaattatagatcggttatttttttttttttctataagattgtctacattaaagttatagtagagtacatccaataggttttgaaaaaattaattatgaataatttacaatattcaaaactaaactccaaatagtaaatttaaatgcacctacctaaaaataaaaaataaaaaataaaaatgtgtaattatcggtttgaattctctttttgacaatataaattattcaaaaatattcaaaatttgataaaatatttaatataattacaatatatattggcaacttctgttattagtttaggggattatatttagacaaatgcaatataatgtgttagattataattgtagtattttttaaaagtgttatactattagaatgtttttttttttttttaaaaaaaaaaagtgttaaaaataataaaaaaaatataaaataaaatattatatttgagatttttaatatttttaaaagatatgctacctatagtgatgcttatgcccctctccggattcttcctaatcctatctagttacacacatagggtaacccaattaggtacaagttacaaatactttttttccctaaaattttcctaacatttggagtaagtacaaaataataattattaggaaaaaaaaacatttttaaaaagaaaatgtttatatatataattatacaaatgaccgtatttggaattaaaattaagagcatattgaaaataatgagtacttaccaagcgcacgagataactcctatgaatgtatacggtctttgaagtttccaacatgtgtgcccgaagctttaaaattttagttactactgaatatttagtattagattcaggaggctcagtttgatttgcagatacatctgtctattaagtgttaacacactattatttttcttaattgtaaaatagaaaactcaattctatcttatgtataatttaggaagaaagaaagaaagaaattgccatataaatatatatatctacaagcaagaaacccacacataccctaatggaattttttgcaattaataatgattttgagcatctgttttgtagatgaaagttcttgttggagcaatagttagagggaaaattattataagaggatgtaattattggtgttttggggtgttgatgagataataatgagctcttgggattcttattatgagggtttaataaagtatggtaattagtttgaaatgaaaagttacaaactgatgagagctccatgaggattattgttgattaaaaaggaca

SEQ ID NO: 186 - DNA, 522 bp, Cannabis sativa
atgtccaagaaaattggtttgcaaattcgaactcgaagcggtggtcatgattctgaagacatgtcctacatatctcaagtcccatttgttatagtagacttgagaaacatgcattcaatcaacatagatgttcatagccaaatcgcaagggttgaagccggagctacccttggagaagtttattattgggttaatgagaaaaatgagaatcttagtttggctgctgggtattgccctactgttagcgcagctggacactttggtggaggaggatatggaccattgatgcaaaattatggcctcgcggctgataatatcgttgatgcacacttagtcaacgttgatgcaaaagtgctagatcgaaaatctatgggggaagatctcttttgggctatacgtggtggtggaggagaaagcttcggaatcattgtagcatggaaaattagactggttgctgtcccaacaaagtctactatgtttagtgttaaaaagatcatggagatacatgagcttgtcaagtga

SEQ ID NO: 187 - DNA, 891 bp, Cannabis sativa
atgggacaagatctcttttgggctatacgtggtggtggaagagaaagcttcagaatcattgtagcatggaaaattagactggttgctgtcccaacaaagtctactatgtttagtgttaaaaagatcaaggagatacatgagcttgtgaagttagttaacaagtggcaaaatatttcttacaagtatgacatagatttattactcatgactcacttcataactaggaatattacagataatcaagggaagaataagacaacaatacacacttacttctctttagttttccttggtggagtcgatagtctagtcgacttaatgaacaagagttttcctgagtttggtattaaaaaaatagattgcaaacaattgagctggattgatactatcatcttctatagcggtgttgtaaattacggcactgataattttaataaccaaatttcgcttgttagatcagctgggcagaacggtgctttcaagattaagttagactatgttaagaaaccaattccagaatctgcatttgtcaaaattttggaaaaattatatgaagaagataaaggagttgggatgtatgcgttgtacccttacggttgtctaatggatgagatttcagaatcagcaattccattccctcatcgagttggaatcatgtatgaattatggtacatatgtagctgggagaagcacgaagataaagaaaagtatctaaactggattcgaaatgttgataacttcatgactccttatgtgtcccaaaatccaagattgacatatctcaattatagacatcttgatataggaataaatgatcccaagagtcaaaataattacacagaagcatgtattttgggtgagaaatctttggtaaaaattttgacaggctag

SEQ ID NO: 188 - DNA, 1320 bp, Cannabis sativa
attcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgtagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaatagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaattttgagagacctaataattacacacaagcacgtatttggggtgaaaagtattttggtaaaaattttaataggttagtaaaagtaaaaaccaaggttgatcccgataatttctttagaaacgaacaaagcatcccacctcttcccctgcgtcatcattaa

SEQ ID NO: 189 - DNA, 1350 bp, Cannabis sativa
atgtctatcttaaattcgaccgtacaaaatcttagatttacctctgacacaaccccaaaaccacttgttatcaccactcctttaaatgtctcccatatccaaggcactattctatgttccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaatccggagctacccttggagaagtttattattggatcaatgagaacaatgagaatcttagttttcctgctgggtactgccctactgttggcacgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaaaagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcacgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtaa

SEQ ID NO: 190 - DNA, 1284 bp, Cannabis sativa
atgtcctacatatctgaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctacgtcatcattaa

SEQ ID NO: 191 - DNA, 1284 bp, Cannabis sativa; identical to SEQ ID NO: 190

SEQ ID NO: 192 - DNA, 1116 bp, Cannabis sativa
tttacctctgacacaaccccaaaaccacttgttatcatcgctcctttaaatgtctcccatatccaaggcactattctatgctccaagaaagttggcttgcagattcgagctcgaagcagtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgtcctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactctggaatcattgcagcgtggaaaattagacttgttgctgtcccatcaatgtctactatattcagtgttaaaaagaacatggagatacatgagcttgtcaaatcaggtgggcggaaggcggctttctcaattaagttagactatgttaagaaaccgattccagaaaccgcaatggtcacaattttggaaaaattatatgaagaagatgtaggagttgggatgttcgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttagtaggttagtaaaagtaaaaaccaaggttgatcccgataatttctttagaaacgaacaaagcatcccacctcttcccctgcgtcatcattaa

SEQ ID NO: 193 - DNA, 1458 bp, Cannabis sativa
atgtctatcttaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaaatgcatgggttgaagccggagctacccttggagaagtttattattggatcaatgagaacaatgagaatcttagttttcctgctgggtactgccctactgttggcgctggtggacactttagtggaggaggctatggagcattgatgcgaaattatggcctcgcggctgataatatcattgatgcacacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaagagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctgcgtcatcattaa

SEQ ID NO: 194 - DNA, 450 bp, Cannabis sativa
atggtcacaattttggaaaaattatatgaagaagatgtaggagttgggatgtttgtgttttacccttatggtggaataatggatgagatttcagaatcagcaattccattccctcatcgagctggaatcatgtatgaaatttggtacatagcttcatgggagaagcaagaagataatgaaaagcatataaactggattcagaatgtttacaatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaaatttaataggttagtaaaagtaaaaaccaaggttgatcccgataatttctttagaaacgaacaaagcatcccacctcttcccctacgtcatcattaa

SEQ ID NO: 195 - DNA, 1458 bp, Cannabis sativa
atgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 196 - DNA, 1458 bp, Cannabis sativa
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaattcatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 197 - DNA, 1458 bp, Cannabis sativa
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 198 - DNA, 981 bp, Cannabis sativa
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatccatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagattag

SEQ ID NO: 199 - DNA, 372 bp, Cannabis sativa
catttgatgatagaatwgcctcacartattcgattmtatcgaytacttaccttggctactyctcttagttctctragaggcctgtttgattgcagcctactgagagcactactcaaagctccaacaggtggtatgggcccatcaagagaggcagtaggtaaagaaacctgtttattgtcgttaagaataataatcatgtcggaatcaaggtacccggcattattcatggcttcataagcttgacckgctgtcatggcrccatcacctatgacagccacwacattattctttcttcctttaagatcccttccaacagccattcctayaccaagatgaagaaatttaacatatgatattagtagctaamwtaaa

SEQ ID NO: 200 - DNA, 2887 bp, Cannabis sativa
atttmkttttgtgaaaaatgataattaagcttccgtgggaatgatttttcaggatcttgaacagctagcagcagagcttagagcagatgyagtcmatacagtatcaaaracaggtggtcatctgagtgcaagcttgggagttgtggagctcactgtagcactgcatcatgttttcaatacccctgatgataaaatcatmtgggatgttggacatcaggtcascacaacattaatgtctttatattaaamtaattacaamccaattgagttattctgaaaaasaaatgaatttttgcagacatacccgcataagattcttacaggaaggaggtctcaaatgcataccattagaaaracttctggtcyagcwgggtttcccaaaagagatgagagtgtttacgatgctttcggtgcaggtcacagttctacaagcatatcagcaggccttggtatcttcatatttaactctwatwattattattattattattagattaatttgyyrcataaatayttargtttaamtkymagttgcaaataaatayttaargttyaatttttgactaataatacttaaattattatttkagaacttttgtaggtatywatttgttaagyattaagtaaattytcayatgtcratttytaattggtrcaagtcatcaaatttttacctttttwaaaaaaataatttaaatatacyaataataaaatgacacgtggattwagtaatattaccgctaaaaattaaacttargtatttatttgcaactgaaagttaaacttaagtattaagtatttatgccgyaaattaatcttattattattattattatwattattattattattattattattatwattattattattattatwattatttawttttyaatccttrtaaattaaagatcaatgatggagaagagtgttcaattgtggtgacagkcatggcagttgccagggatcttctgggaaagaagaacagtgttgtttctgtgattggagatggggccatgmctgcaggaatggcatatgaagccatgaataatgccggctacttggacgccaacttgattgttgtattaaacgacaataaacaagtttctttaccaactgctactcttgatggtcctgcaaccccagtgggagctctaagtggtgctttgactaagcttcaagcaagcaccaagttcagaaaacttcgygaagctgcgaaagtaaaccttaaagccaacttatatgactttctattttgagtttttcttttrttttattttacatctcrtgtcaagaactgatttacgaattttkatctttcatagaccatcacaaaacaaattggagggccagcacatgaagttgcagctaaagtagatgagtatgcyagaggaatgataagtgcttctgggtcaacactctttgaggagcttgggttgtattatattggtccrgtggatggrcataatgttggagatytagtcaccattttygagaaagtgaaatcwatgccagcgccaggaccagtcttgatccacatcgtcacagagaaagggaaaggctatccyccagctgaagtagcaccygataaratgcatggtgagacaatagatatttagtytatrcacttttttttcttttgagttcttgtcaattagcaragtawtaatccytcaatgatytcaggagttgtaaagtttgacccracaacaggaaagcaatttaagtccaaatcgtcgacactttcatatactcartactttgctgaatctctaataaaagaagctgaagaagatgacaagattgttgccatacacgcagcaatgggtggtggcactggtctcaattatttccagaagaaatttcctgatcgttgctttgatgtggggattgctgagcaacatgctgtcacgtttgcagctggattagctgcagagggtctcaaaccattytgtgccatatactcatcattcctkcaacgaggatatgatcaggtaasaaatgacaayaayattgtttatcaacttcaacactsatgagtttttttttttttgccactcatattcacmatctttgaaattatttgataaacacaagtggccatttmggactagtttcaacctgttttgtgtgtactagaaaytttctagtggataaaagaactgaaggtgttttcattcaggttgcacatgatgtagaccttcaaaaattacctgtccgttttgcattggatagagctggcatggttggcgcagatgggcctacccactgcggtgcatttgatatcacctacatggcctgcttgcccaacatggttgtcatggctccatcagatgaggctgaacttatgcacatggtggccacagcaacagctatagatgacagacccagytgcttcaggtttccaaggggyaatggaattggagcaaakcttccagytaataataaaggaactatacttgaggtaagcatagatcattatagaagtcttaagaaaatattcagacttaaatattgagattcatctctataayttttattcytraayagattggaaaaggyagaatattaatggaaggyagcagagtagctattttgggttatggttctattgttcarcaatgtgtggaagctgcaagcatattaaagaaacaagacatttcagtgacagtagctgatgcaagattttgcaaaccattggatacaaatctcatamgacggttagccaamgagcatgaaatcctaatcactgccgaagaaggttctattggaggctttgggtctcatgtgtcacactttctaagcttaagtggacttctwgatgggtctytaaaggtagycttcttataaaagagaaaattcatcatagttgtaacaatattcc

SEQ ID NO: 201 - DNA, 4311 bp, Cannabis sativa
aaamggttacaaaaaakaakttttttwaaaggatacagwttgaatggytctgaacttgttatgccctgctgargtgarkgttctatccgwtttggacyccatcaagtccacctgcttcccyaagctgtgtccaggttttgcttccattcttgatattctctatttggctactcagaaagtasgagggagattacataaaatcaaggctcwtattttttttagtggggaagcattaagttatgagcycagctgaagagggatgagtggttttgtcaatgctttctgaaatatgacaaagaaaataaagagcaaaaatgtcaagytttgtgaytttttttccttttcttwtagtttgaagctattaacttcttgcttatacyraagaattcttgtcccaacaaactaaactgtgttttgtgtccaagttttcattgtttagtatagattttagaagtgattcttatgttgctyaaaacacrtaattgattcttatgttgcctatatttaatatgattattaccagcattatttggstttgaaccccamtgagggtattatttatggtgttttgatgtagttgatacctyagattktctgtttttgtttwtgctgcwcccagactatgacagcttgagtttatgccattctcttattcgttttctttaggtggaagtagtttccatagaaaggattgcagagtaccacttagaagargagttcattgttcggtgcagcaacctcctmcagcttggccaggaagagctattccagaggaaggtctctgtaattggaatgtcccaaagcctgtttcaattctaggctccactggctctataggractcaggtacaaaatcaaaccaaaagactaaggaaagaaaaagagtgaattttgtctgtgtgattaacctgggaatcatggtttaaaggtcctatctkaatgcccttagtcaaaaatagttcctgtttcakaatatgacattaaaggcctagtcataaactttatatgggaaaaggattcacaaaaacaatttgtaaagatttaagttgtgaaaaaaaaaagatgggtcctttcattaccccaatcatgttttagagttacttgtataaatatttttttagcaacaaatgaaacataacttwtattagccaggtttctgtaacttgactccagtcagagctacctatttcaagagagtttgagttctctttcttcagttcaggtcttttttgtytgaaaacagaagacattttctgtatttgattttaacagtatttggtttcttgccttattattattatytcttcatacattccttgtaactagagtatgttgattttcakacactgaacattgtggcagakaayccagataaattcagagtagtgggacttgcagctggttccaatgtaacccttcttgccgaacaggtattctgttaacytgacctaaattcacagatcataactatgatcgttagtgtatgaagatttaaaggttcaaattcaaaaacttcccatatatatgttagtttcatyattttgacaggtgaagagattcaagcctcaaatagttgctcttagaaatgaatcattaattggtgaactaaaagaggccttagctgatgtggaagaaatgcccgaaattattcctggggaacaaggagtcattgaggtgatcttgtytctaacagtctgatgataaaatrcatcttttcttctagattattattatttttttattattattaatgccattctatcagcataaatatttctaacagcataaatatttctatttttaggttgcccggcacccagatacagtcacagtggttacaggaatagtagrttgtgctggattaaaggtaagtccataaaggtttagatgcacactatttttatagtatgatatcrtatatatgtagaatcaactgaagtgttcttcctattagctttatattttyttaccttttttgaggaggtttatttaaratgtgkcaagcatatttctggtactattascagttgaagtatatcttgctktttcttttttcttagcctacagttgctgcaattgaggcaggtaaatacatagctttagycaataaagagaccctgattgctggaggtccattcatccttcctmtagctcacaagcataacataaaaattcttcctgccgattcagaacattcagcagtatttcaggtacatatacaaacraatttcaatggatcttgtctaccctattyattatttctctcacatccattagactatrttgtcaatatgtgaagcattggtaaatgtcaggaccaatawtwtcatggyattgtcatcattgtgccacatgttttcactatrgttcaytagaagtgaaggttaacgaaatctgttttcttcagtgtatccagggyttgcctgatggtgcwctacggcgtgtcattttgacagcatctgggggagctttcaggtgtgaaataagtattttaattgaatttggtagcggaatattaaaattgttttaggccgttgataggacacggrctttactacrkgctaytgcatcagyatttaagttatatattgatttggtgctatgatttacattttgaagttaaaaarcttgcataaattattttttwaaaaaaacatgcttgtagattgtcttattttctttgaataagaawgttaaaacttaaaattccataatctctygtctcgtgtacaagatctatgtgtattatccttttcaaaattctttggacatgattagtcattwaacaagtatctggttagactgccaatkttggtgatttcaatttttgaacccaagctcatttgttaakattctgatttggtgttatattwwaactcctttatgacagagatyggccagttgaaaagctaaaagaagttaaggttgctgatgctctgaaacatccaaactggccrggtatgggaaagaaagtcactattgattctgctacccttttcaacaaggtttgatacttgtttycattgttttctaatgtattttctctttcattttcaactgcgagcagtaaaaaaaaggtgtctttgttttttctttttwtcaattctgaaacagggtctggaagtcattgaagcccattatctattcggagcagactatgacgatattgacattgtgattcrcccagaagctattatacactcyatgattgaaacacaggtataaagctaaaactagtagtcttggttcttcttttgctagtttgcaataggactgtaaattacaaaatggagagtctattatacaaaattayggwctcttcaatttgtcttgcaggattcttctgttctggctcagttaggrtggcctgacatgcatataccgattctctatactatgtcatggccwgacagaatatactgttctgaagcaactttacctcgacttgatctttgcaagtaatcrtctttttccataatatttttccacaatctttttccgyatgttttatcgaaagatatgcctacagtaagccaaaattcagcttttacatgtaaatccaacaaagtctactgctgacctttwtatctaatttgtgrctttcagaaccaatgtgaactgaggattttcccccctttatygayatccgtaattattcaataaattagcatatttctgggtttttttaaataaatttctcaagtgaaagtcatgtttattctttcctagatgcctttttcatttttattmttttyagttcttttcctacaaaaaattygggctgagkgtataaatggaatcracattgatgcaggcttggttcgctgacctttaggagtcctgacaaccagaagtacccatccatagatcttgcctattctgctggacgtgctgggggcaccatgactggagttctcagtgcagccaatgagaaagctgtagagatgtttgttgatgagaagtaggttttctatttcatcayctttcattgataagattgtcacttcaawtcccaactttgcactatctatcaacacactgctatctgttatatcttcctcattccaaattatattccacccatgttttcagrataagttatcttgaaatcttcaaagttgttgagctaacatgygacaagcatcggtcagagatggtgacttcaccttctctcgatgaaattatccactacgacttgtgggcacgcgagtatgcaactactactttgcagagytcttccaatccaaaacctgttccaacggcataagaggagacaacttttccattgttgaccagggcagcaaaactttat

SEQ ID NO: 202 - DNA, 4248 bp, Cannabis sativa
tattttcagattacaaagaatgtramagtacarcaaaaaatraatayttatgcaacagccttyacagagttcatactcaataytctctccgcaagcaataaatcatctrgagtrgtaacctgcaaargatttgaccrctttaaatattyattttgaaagggaccrctttaaacttgattrmtttttatagaattatagsttccatatgaamaacaaawgtgaaagtttagtaccttratgttggtgtaagcaccttcmgttatatatacaggatgtccaaggtgttcmactatagacacatcatcagtcacttccagattttccctgtagacaagtttaawgcatactryatcaaaatttaaagwgaaagtacattggtcattttacaatgtgtrtgaggaatttcttctaaccaaagtaacctcactgttcaggcttgaaattaaatwgagatggtggaagtcaattatctaacttttttttawgaggtggtgaaaagatragtggttacctatttacaagctcaaatccttccttgagcaactcgggtttgatcacctgtasaagacaattgataggcatccatgaacattgaccratcaktattatttgaartgatsttaaattaygaaatgaagcaagaaragaamarrarraaamraaaagaaacttaaamttaraattaaaattaaaaacctggggtgtctgcatttmccaaagttttttcctstccaacgttttagttacaaaagatgcattgttygcctgcaccataattattaaacaactttcaamawaamcaaggtrwaaatattagkrgayyttacatttaatatggaamtcrtgcwattaatgcctataaaacwagatargtagacataaatgkttatttcaaaggcmggagctagrtmttgytcytgaatgaaacacrtttatwataatatcaaaaytgaaatcggcmtaaatccaakarktaaaacaactwatcatataaaggaaataaacatggktttaacwtgcattcactacctctttgattgtagctttgacaggaacaccaagtacagctgctycatgcaaccaaccatcttcaawgacctaatatgcaaaccagtaaacttaktcagcctcttagagcagaataattcaygraatgtattcaaagttcaaactttgaagtaatgttttgagataatttgcattatgtataattataattataatttcttatgcttatakttggcttattaaatggacagtaagtcttytcaattaactttgaaacgtgcccccaacatgcctgcatatccacagatgcaactgagaatgtctcagatatwgtgtcatagcatscttgcatttccacgaatgcraccaagaacttyccagaatactctgagtgattctttagtgaatatgaygtgacatgtttcttacrcakcatttctcayatttgawttgggttgtcwtcacataaaataagatttttctatgcaacgcaaattttacaagtaatggtcaaacaattccmtgaatcaatcaggaaaatatgcaaatactattagcaystacttracaaggaaggaagraagaaagtgatggtgcaggatttgatactgaagattcataacgtwcttgtacattaaaaattaccagaataaacttaaaactaatccaatgagttataaagtgcataaaatatgttctcacacctttttaacttcttctgttgttaccaaaggtctagcagaatcgtgaatgcacacaagcttagagtttggatcaattkcctgtaaaaagaatatgcagcactactattaaaaggscatttaatgtagaataccccaagcataaaaccaacatraccaactgcaagagattaaataatatagccactagtacacgtcttaagatgcaattcagaactatatgttaaactggtaaaaatgaatataatatccagataagggaaacaaacataaagtagttatcggtgtatcttatgctaaagtgacatccaaatagaaagaaagcaggrcaamgaaacactaatctgacraatggaccacygaaaatatatttgraaagaatgccaaatttaaaataggaatatggaaakcaaaaarttatgcacctgaagtccactataaactgaatcctgtctttcctttccaggcagagcgaatttaagtccaacatggatcttgwctttggaatctgaaacattacaagttgctaagtggckttagctccaatttacayaaaaagaaartacttttttacttttaagkaaaatgcatgtcwcgaatatgaagccaattacagagagagagagagagagagagagagagagagagagagagagagagagagagagagagcaccttcaaagatatcctcgtaagakggatcacaaactacaacaatttcagtcacttcaatcattttagaaaaagtgtagaarstgmagggaaacaactcagaatwatcctcaagaacacaataacaaatgaataaaataaaaggagaaagaatgcgcgaacaactcarcmattgtctaaatggtataaacactataaaggtctcaactaagccactaggtttggtcttctggtgaggggagttgggtattaagcacaattgmgatgagaccaaggtttgaaccttggatctcgtagtgttgagagattcctagtattaattaagcaccatgtrtgccaatgatctaaacacktctcatctaggaccatggaatctagataaataccayaaagaattaagaaagttacttttacttccaacaagtcataaaaactcattacgytcamaacattagtgygctggaccaaactcaaagctcaaatgatacattttgattcattawtttaatgataaatatcaatrtgaaatcwttacacaacamaattgttgcatgttytatggtttggctgattttttttctcttgactatttcaaatccgaagtttwaacttttaaaagttaaactaaagscagkattrccatacgaacacgayttcatttcgaaattygtaaaaagaaagctgaagtatcatctactagtgcaaattgaaagtaaaatwgagtttmagtgaatttacctrtacagtgcaatwggttgccctaaaagtggaagataytgctttggcatgttggcctattgtacaasataacaaaaccatacgatggtagrtgagaaatsaacascctgcatgcaatagggacttacgagacatatggraaataaccagaaawrtataacaactatattacaagacatccttgctcaaaggaaactgcttttctacaatagcagtggagagttaagacaaatgtatgacyctyytaattttgtgatwaaatgtttcttaatctctcctgatamaataacaattttcaggaaatggaatrtaatartaaattgaattgaaytgagttaatacaataacacagatcaggtctcaagkgaaatgggtcatagaggaaaacragcaggacttacccccatccgtttacccttcccacctgccagaagaaccaccgaaacgcttttcttttccacaayrccaggcttctacattgttatatrtatatataatataaatratagaacctattacgacaaagagaatacatttttatatctgatagaamcmyaaatmatygttycgaaaagaataaattttgataagtcatagaattcgtmtgtctgaatcttacttggycaacatttgcagagcagcgagcgaaccttatcttggaaattctkcatttccccctgaatcccacctcatggactaaacacccaagaagataaaaaggaaaaaagaraagaaaaagtgatcgtaaakacactkccakwcaaaaatgagagygcraaataagckaagtttgstggcataataaaaacaacccaattyccaattcgtgttatgatgaaaacartgatcatgtgaactgattttgcggggttggagaagaagcwygaaatacttgcctgggmatcgaacaatggattgagargatgggcgaawagaaggaaatagtagaggagcagcawtgtaggaagaagaagaaagagaagsagaagagaaagtaaggtccattgcaagtaacgccatgttgcgttttwctctactgactttttttctttycggaaatggagaaaagtgttataataaattaaggtatrgaagttggkcagccacacacacacaagcatttawgggc

SEQ ID NO: 203 - DNA, 4029 bp, Cannabis sativa
tatggtcattttacwacacrcaytaktattattatgattcattcttattactcgacarwggaggaaaaattacgagagaaatcatcwgaagggctacaagcgctagctgaagcwggttctttgtaccattcatttgcttctcgagtcagaaagttggcctctgcgtataacaaaacaactttatacatcttcattttttagttatttatttttttawttttttttttttgagggaaataaaaatcagtagcyagagaaattaamttaamrgttgacttgcrtatctctaattgttctcytctaattctcagtggcttcactatgggaaaaataaccakgagactgaatggtctttgamaatcataraagattataaccagtctggtcttgcaaataacacatgactaccatyaatataaccttaattcattttcaattsggrttataaaaaaycctaaaacaagaatctcttaacttggtatttccaactttgatatttcataattyggataartactcgaaattagtactmmggagctctttaatttttcactttgcagtgtgtaagacaaaataatatastatagcaytttcaacsacgrccttccttatgagaggttagatcactaaaattactamagatagccatgacaaaatatccrcattcatttgktasatttccacaactagaaaaaaawwackaggtaaaaagcttagaagactaacctgacaaaaayayatccttgtagycctcatcatcatatataaacwgasgtggatyyggggaacygaccccgacaatrgtgcttccactgcagcatgcacgatgagaaaatryaaaattcttrtgacagatratacaatgaaactayataacaagtyccwyattcataaaattcattgatgcaagcatcttgatgwagattattwatgaacmcttatatacaraaggacttgcaatcartatcatcagatgattttttttgtttgtttctatagttacakggtyactaactaaatcttctcttaagaaatctaaaccytattgctcrattttawcagaaatatttgaaatttccagatgctaactgcacgatttarttataagcttagtrgraaaatgatcatccrgwaagaaaaatcataacaggagacagayactctayaacagtatggccaaatggtcaggtcaggagaagtcartttcttacttacccggttttssatacccaamgagccagatgttcctattgactttactttctaataaaattcatatwgtgaaatgcagtttaaacatcccttatttataccatctgaacycatatacagtttaaaatccatagctcaaaatttccacattsagcatatgaacatatgagcaacataaagccatragagctagaaakccagtatttatacctcccagacataaaaacagcatcatattgtccacgactagctgcaattatacgctgtttcaatctcttaagrgatggaagaacttcaaaggcaggaggttctgcaaataaaaayatcattagacatgtaaagaaacaaggaatacttctaagaatgcacatcgaagttggaaacttgttgagagagactcaytaacaccaacgcccwtwtggmaaarcaatactggcatacgmacccagtacaaactcaagttagagtaaaaaagaatctaagcagtaaaactaaaatattttcttgacctccaatgtgtgttgagatatttaaaacatgaagcttcgacaatcaggyacataaaatattcaaatattcatgaccatccaaatawtcrtagcagcrtatcctktaataggtttattgcagtgtagtatcacaaatgaaaaggkaatatttacttacctaagtcattgatgcagacatcttgacttattccatcacttgagatcttgtggagcaattgtaaaggatcrctattactggttttatccaacygaaaacgctgcaatacacaaaaaagttgccattaaaatgtawcaaaagaaracgatgaaatattcaccatgttaaaagagcattaccttataaacttcrgctgttgaacatgctyctgggggctttatgagaaccatgrgaatgtttaatggtacaggtggtagaatatcctgaacaacctggacaaccaaagaacattttatagcagaattcatcacaattactagacctctatgcgaactcctttatttagattagacaagcatacataggtaaacaaataaaaaacataatgtcagattcaaactcatagactgtcactactgaaaaccctatgtagttatgcagtagtaacattaaacacaagaacccatgacatacagaattatattgcacacactgcacagctatatgcatctacacacaaattcctaggctgtatactgtgttccctcattttgactggttcygaaagccgatgcaacttctcaaatatgagaaaaaagagcygagagaaaagtacaatagggctttccatttaataaaaaagacagtagccaaattmtggatttttttygaaactggctgcttgctttaccctacaatatttgagcatgaaactatggcaattggtacccaacagggattcaaattatggaaactttrgcagattcatctttggcaatcacagaaaaaggattaatagattttcaaatgaatattgagttgtaaaagaattataatatctttgtttgaactacakacagtaagataaagacctacctcacctcgacctgtacaataggctgccccttgggagaaaaagaaaggaacatctgaaccaatctcacttgaccattcttgcaattccttttcagtaacaagacaaycattgaactgatttgctgcccatagkgctgttgcagcattgctgctcccaccacctagccctgctccagtgggcactttcttgtcaagatgaatctgccaacatgttaaatgacacaaggtatcatcaatcaatggcagctttctttaaaaacaaaacacaagacataaaactgttttakrggtctcttacccaaaagtatttgtttgtaccagttttctttcggtaaagattaagggccttgataatctgtacaaaagaaaaaaagtcctcttatgagtgattaaaaacatcatmatgmaacaaataaaaagcagcaaaggaacattcatcccttaccaaatttctatcatcaagtggtaccccagaggcattcgttgacaaagaatctttctttgttgaaggagacaaagagaacttaagcacatctccaagacttatcacctaaaatttcaaccaaataatcacatatgtttcatatrttttccaacaacttraacaaatttaagtaaaacttacgtgaaagagggatgccaaatcatgatacycatcttcccttttgctagttattctcaagaaaacattaatctgcattcatgatgagacatcaaaaaactgctgawattagcataaatcaagaaacccatttactaaaaacaatgacaaaccttgcaaggtgagaaaagagtgagcctagaaagaggcgcttccttgtccactacatcagccaatttgtttatcctttcatcagyatcataaactatctacaaccaccaaactaatamttaaaaaaaggatacaaatttatacacaaaaggtaataaatgatcaaaaagaataatctttacaagataaagactgaaaawtttgaagtcaaaagggtgagaaggaawkggagattccaaaacaaacctcaacttgtcttctttcagaaaccacaattytgagagatgaaggcctcgaaattctagatttcgaaccaaaaaaacaaaacccatttgagtttaaggaagagagacccctgttcctaaaaggattgggagaaagattaaaaacgttgttgtggcagagaatatgagaggaagccattttagttgtggagaggtaagtatgaactmactaactatcaaactgtgaaaaaatggtgcatttraagacttgggattyag

SEQ ID NO: 204 - DNA, 1944 bp, Cannabis sativa
actactaaccacatgtttttgayaagraytttccaagcatattcttttctaytttttcataagaagaaccacagygtgagcagcgatgcttcgattttccmcgagactatcraccttttcrtgagttttygctttcagattaacaactgcagggtcagctcctagaagttcagacaagttggctctgatagcttccttgtgtggacttaactttggtctttgaagaattaatgttgcatctaaatttccaagctcataacctgcctcatgcatcagtctcacctraaatayagataaaacatattcaaacatgtttatattgtctctcaactamtaatagacagctcagagtkartggttttcaataattgtgttccattgtttgtgacaaagagacattgttsagaaaaacttcccaagtataattctwawtgataatttcaacaagtgtgtgtgygtggatgtaaattctacagttaacaattcattattrgcttcaaacaaaaagtcagacctaracagataaataagactttgtcaagggttaatcccartcycaacagtctcaatcatttgrgtttgtgttgaaaattgataaaactaaatatsaamtacttagttaacatwrtgaaaagagaaakaaaaacatcatttcttgcttagagtttatgttatagacagwggamtaggcaaacckarctttgytgaaccactatatttcytttgtgtcattactttytactttctttttcattgcttattattygagtattcgtactttaagmgagtaatcrcygttttgstgtgattgttcttgscttgctcgaatastcgcacttttagtgaaagatagaaaawcatyatttcttgcttagagtttatgtttgaacwgmtaaaargmaayatagcayaaastcaattcaatggtaaaaatytgyayatgactgaagatttsataagcacaawacwattcaatcaagtgtaawatgcatagaaaaatcaaacaaaattggttgaatgaaacataacaataacaacttacagcttctttgatgaaaactgatgatgcagcccctttccatttgggatcagaatcagggaaaatttgaccaatatcaggaagccccaaagcacccaaaatagcatcaactacacaawgaagcaaaacatcccctacraacccaaatcaaagaatatatctttaaytctcttktsacaaagaaaatstaagaaarttcaagtttttatwttctcagcaaccaaacagtataaaatttaagagttcttctacactgcacctcttagtgttaaacttkyaacagactatttgaygagttaaattattttgaatttctwtaaattatamcaaatataagattaaaactatctaawtatggaaaayamagagtcgaaatttctgtaaatttagagcttttactaacttaatccaaaaattaaaagaaacccatttcagatacraatgattaccatcagaatgagcttcgcaacctttctcrtgaggtatattaataccyccaattatcaaaggatacccaggctccaatcgatgaaggtcaaacccatgaccaacccgaaaaggaagagcctttgagggagtarcagatacctcagacggagcttgctcagcgtycamagcggtggtmgcrgcggccgatayagaaggcagagccraaagcggcgtcgttttcgattgtctyagagatgaaattgaggaygatgagaagakatttctgggaactgcgacygtgaaatgggagtgattgaatgatttkggggaggtattggagtagtgtggwggaagagttgaagcacagagtgktgttgccgtcgccgccgccatggccgtcgaaggtggaggtccgtgggtttgtggtttggctttctcttcttagctakttgttaatttttttttttttttwaaaa

SEQ ID NO: 205 - DNA, 5363 bp, Cannabis sativa
ttttcttagctatcatattgagcttgtgttatggaattttgaaggtgagcttttgagtttgtggaaatggcttctggagctgtaccagcatcaatttcatgtctgaaaagcagagactctgscttgagctttgctaaaagttctkattttgtgaggctttctgatttaaagaggrttggttcatctagaacaagagtttcarttatccgaaattcraatcctggttcagatattgmtgaacttcagcctgcatcaaaaggaagccctctattaggtactratttctttgatctttcartctttgttgattatttaaactcatgaggccttagaagaayaamagaacatytgtgagaagaggatatatctgcakaaakctaakcttaataactgactttttaagtgtttgttatgttgaaaatggaagtaataaacaacttcgaaayaaattyggtaggtctgygtttgttgcttsttttgtttttctgatgaaaaaatcgtttggatgtagttcctagacagaaatactgtgagtccttacacaaaactgttaggaggaaaacgcgaactgtgatggtgggaaatgtggctcttggcagtgagcatcccataagaattcaaacgatgacgacaaatgataccaaggatgttgctggaacagttgaagaggttttataatttctctaatcgaaaaatattacaattattcattgkctcaactgcacaagcataagtttgatactaattagaatttattagtatgacatttagctcacttttgaaggggtagttagttagttcttgttaatccatttttttttttctattttttttttggtggtggtggtgaattcttmtggaatttcgggttagtagaaacatgagtataacgaattttagaagagtttaaccttgtcaatagttcaaaggtgggaagtaatcactttttatatagaggcggtttacattactgtgtctattcaatttataggtgatgagratagctgataagggagctgatattgttcggataacagttcagggaagaaaagaagcagatgcttgtttcgaaataaaaaattcacttgtgcagaagaagtaagttwkgtttattstttkyttattggwtttygaaactttaaggtacatataatttgaaggattgattatgttactatcccatttgtatgtgcagttataatatmcctcttgtggcagatattcattttgctcccccagttgcattaagagttgctgaatgcttygataaaattcgtgtcaatcctggaaatttcggtaaaggctttgtgattcagttgatttatttgtgtagtraatawakgcatttcttctggattyrtctaagcagyttgttacagmtgacagacgggctcagtttgagacrctygagtacacagacgacgactatcagaaagaacttgagcatattgagcaggttaaatccattcatwatcgatttgtgatcattgtttatgtaaataaatcatttgtccataaattyattgaagctttcttttagyatacatgtgattgccccgattgaacttggaaaataaaagacgatatatatttttcattgtttatgtgttgtttctatttctgaaacaaaatcggttctttctcacttcacaccaggttttttctccattggttgagaaatgtaagaaatatggtagagcaatgcgtatcgggrcaaaycatgggagtctttcagatcgtatcatgagctactatggagattctccwaggggaatggtaaatwwtttttttttctywttcttgtttttcttgtasaayaaaayttaatggtsatacttgrcttggcatycaccatcgaacaggttgaatctgcatttgagtttgcaaggatttgccggaagttggatttccataattttgtrttttcgatgaaagcaagcaacccagttgtcatggttcaggcgtatcgtctacttgttgctgaaatgtatgtccarggctgggactatccacttcacttgggagttactgaagcgggagaaggtgaggawggacgaatgaaatctgcaattggcatcgggacccttcttcaggtacctttctatttctgattcacgatcaacggagtcggtacacagatatttctkcrcttcaatatgtcrygaactttaaagccatcrgaattctttgatttyggtcggttgtcttcctaaaagccatcactatgtttcgaacgcwttttacaatttayaattaagcaagatscatgtcatgcatatcaccatagctrgtgtaattgaatkttgaawtgaatttgcaggatggtttgggtgatactatcagggtttcactcactgaaccgcccgaggaggarattgatccctgcagaaggttggccaatttgggtacaaaagcagctgatcttcagcaaggagtggtaagatcttcaaatcccttcatttaattaaagacttytcttcaaatcaatatttagtacactrtctcaactttctcgataatatgatttcgcttcagrctccatttgaagagaarcacagrcaytattttgattttcaacgacgaactggtcaactgcctctacagaaggaggtcrgcctttaatatcgagagattygcatggcatrttgytcaaagtttaaaactttttaacagaaattgatgcttcggttttacagggcgatgaggttgactatagaggtgctctgcaccgtgatggttckgttctcatgtcagtgtctctcaataacttaaaggtagcwcacarwagcacaacatccttccatttcgattttctttcttttgtcctcwtttcgggcacgrgagagacsattctagaaaccygaaaatataacygtttktcccttttccagatgcccgagctmctrtacagrtcwctagcagcaaagcttgtcgtcgggatgccatttaaggtctatatattttccattttataaccgtgtgaaatcgttatttygcaaagctgaatatccatttgataagttttygtttaattgattwtaggatctggcaacrgtagaytccatcttattgagacaacttccacctrttgacgatgacaacrctgtaagaaatmtaccgaactgyattttttcgtattagacaaagtcatwtgatctgtgttctaatkttcttcgcgtcttaatatgtttttgcattgttrttttaatcgatcagmgaytrgctctyaaaagattgatagacataagtatgggggtcataactcctttgtcggagcagctaacaaagccattgcmraatgctatggttttggtaaatcttaaggagttatcatctggtgcacacaagcttttgccagaaggtaaaastttyggattatttwgctagagaagraagatgtatatactaattctgactaatacatatataccgattttgttccaggcacgcgtttggttgtatccttgcgcggtgatgaaccttacgaagaactggagattctcaaaggsgttgatgatgttrttatgattcttcatgatcttccgttcgatgaacataaaattagcagagtccactcagcaagaaggtaagtaatwataagttagtgtggcttgtgaktttgaaaccrctgcttttyaaggarattrgactstgywatcatacaattattwacttattagtacttttagttatgaaaaaaaaactkaggttgcgttcggtcggaagraatgaatacatcaraatagaawtagtaatragaatgagaatgagaataaaartaggaatttgaatctagtycgggtwtgggctatggtagtccgyggttagggtcyacctaatccaatmgtcycaaaaaaatatatattttttaaaaagtataccatattttttattggttttcgaaaatactacatttyattaatacaakgcatagtaatttttcttttgaaaatattttacaaatamacaaaaayaacaaaaaattataaacatgcggttgtatagattgattttatttacagaaaatacrgtcttttgatgtattttcatgttrtactcttgttaatytgttattgartttttgttatttgtatkttattttttrttgttgttttgatgttattttyckgttacttttatatagttttcttattkttttagtgttgtytttataaaayactgtamaaatgtaaaaaaraaatctttraacrtaaaaatgtaawaaatttacaaaaaawaaatgtatattatgtaattatccattttytttttctcaaggcacrtccaatcycagggygacygtagaataaaatttaatatgcataacaaaartywataattmttttttcrtcttgcattagaatgaycttactsgcacragattawtttatttaatttcttatattaacacmcaatatgttactttatattggctgcagattatttgagtatctatcagataattctctyaactttcctgtaatacaccacattcaatttccaaatggaatccacaggtatgaattgaattctcttaatttgactatgaaagattatgagatattctattcagtcatgaawtttgacwtmaaaaccgttttcacgcagggatgacttagtcatcggtgcaggtagcaacgctggtgcccttttagtagatggactcggggacggtatcctcttagaagccccagatcaggatttcgattttctyagaaatacttctttcaacctacttcaaggttgtagaatgcgraatacaaagacggtaagaagcttttatcatttgaaaggattcgaaatatatttsggaagaaaaaargrtccaacttttcwwaaatgtcgtcgcaggagtatgtctcgtgcccatcctgyggtagaactttgtttgaccttcaagaaattagygcagagattcgagagaagacatcacacctgcccggtgtctcagtaagtactcgagtccttcgtttttttkaaatattattcctttgtatttaactcktttygatmttttcgtatgatagattgcaatcatgggttgcattgttaatggacccggagagatggctgatgcagacttcggttatgtcggtggtgctcccggaaagattgacctttaygttggaaaggtawataattatgtyttcggttttatatttcgaaagaayrtatggttraaatcrayatggaaatttrsrttatgttttttcagacggtagtgcagcgtgsaatygcaatggaacaakcraccgatgcattgattcagctaataaaagatcatggccgatgggttgaaccaccctcrgacgaagaatgaagagtgtatcatcgtcgtcrgtagatagaaaataaaaggrcag

SEQ ID NO: 206 - DNA, 2307 bp, Cannabis sativa
ktcatccgatgckccggygagtcatcttcaacggcakcmgattctgatttcgatgcgaaagtkttccgtaagaacttggtcmgaagcaagaactacaatcggaaaggttttggccataaggaagagacccttcaactcatggayagcgagtacaccagtatgwtgttgtgcttmgattcctcgatytgtttatgattaaaggattwwttttttyktttkcktrattgaataattaatttacttgaaacaaaattcaggtgatattataaagactttgaaggataatggraatgagtacaggtsrgggaacgtgacggtaaaattggccgaagcatatgggttttgctggggtgtggagcgagctgtccaaattgcttacgaagcaaggaaacagttccccgaagaaaakatttgsattacaaacgaaattattcataatccgacagtcaacaaggtttgatttttttttttwattagaayttgtgcttcgkcttcgtartggctcaatttatatgtttatttattgttattatttggatagagactagaggaaatgaaagtggaaaatattccaattgatgaagggagsaaacaatttgagattgtaaacaagggtgatgttgtgatattgcctgctttyggtgctggagtggatgagatgttggyyttgagtgataggaatgytcaaattgttgataccacatgcccatgggtttccaaggtaacctttttgtttaataawagagatttttgaattatgggattmtgtttaaattttatgttattaatgggtttggttacaaatcttctgttyktaaggtttggaatacagtcgagaawcataagaaasgtgaatacacttccattattcatggtaaatatgctcaygaggagactatagctactgcatcttttgctggaacttacattattgtaaagaacatgaaagaggtaaacaactctcttatgacaaacaagttttatgctttakatttwaawtgaaacctttatggtcttmaattcaggsaatgtatgtctgtgattatattcttggcggtcaacttgatggatccagctcaacaagagaggagtttatggaggtgtgttttgccwattcactttgattacttagttgataattttcttgacacaawtttttcaatagttttrtaacattcrttcttgrgstttgtctgcagaaattwaagaatgcagtttctaagggatttgatcckgacaaacatcttgtgaaggctggtattgcraatcagactacawtgctcaagggggaaaccgaagagattggttagtttttgcagctrgttatgytatagatcttgamgaattgccrctacttgtttgcaatgtctttttkaacttatattcttwaatttcwgggaaactggttgagaggactatgatgcaaaagtacggagttgaaaacatyaatgaacacttccaaagctttaacacaatttgcgatgcaaccmaagtaatamgttcctttctcttcagttcatgtawtcttgtcyggtttgttacttctgagawttcatyttcctcgagcaaaacattttactgtcaaatgaccattwtgttttctttgtttgtttgwwcttctgattaattgaygrcattcctcttgtgtcatgtagtctgagttaattttttwatgtttttctttaggagcgtcaagatgcaatgtacaagatggtggaggaacgtattgaccttatgttagttgttggaggatggaactctagtaacacktctcatctacawgagattgcagaggaacgaggtattccctcktattggattgacagtgaacagagaataggtcctggaaacaagatagcctacaagctaawtgtaagtttggacagataagactaccattgcaatctaattatttcttgttttctgcttatgtactagtttagcrgaactcytaagcaatggttccttctaatctgttgtcctgattgttgtaaaytccttattgtgaattttctggtagmatggagagttggttgagaaagagaactggttaccagarggtcccatcacggtcggtgtaacatcaggtgcttctactccagataaggtgagttctttataccaatcaaaawtwgcaaaccgcttcaatgaaatcataaaaarycrtctccattcwactcacaakgaattttctttatcaggttgtggaagatgttctcatcaaggtgtttgaccttaagagcgaagaagctttgcaagttgcttagtttaagttagactcagtgtttatacatcaacagacagcgc

SEQ ID NO: 207 - DNA, 2919 bp, Cannabis sativa
ctttattggatagaaagacaattgcgccactaaatgactactaaggctattaagtcaacttgtgaatggttttcatatcagcaacttccttaagtgtgcctttctcaacatggtcccaccacttgaacaagaaattatccacaaccagtctgaaccaaggggatagcttcaagcctccttccccagcatctgctttcctcaacaactctttcaactcgtcccggttttacatacttgatatcagctacttcatctggrtttggattgacactaacatcccggacgatgaagagcaggtaatcaactgccaaacaatggaataataaccaagaagtkagcatgacaagtcaaaaaaacagcgctcccttaatyactctcttaaacttaacaaatttcaacctggtgycaawagctcacaaatatctggatgctatttatttatttatttattttgcatatgatgtataataaagtaagaattattacgttcatgctcgccccatttgccatcagaaggagctttgtacagcatcctgcctagtggggtaaattgatcaactggcacatcttcagcaggaatacccagctcatctaaaagctttctctgtgctgcattccttgctcctggtaattaaaaaatataatgagattttgaagaggtaagctttcattggataatcaaatatatattctgcagaaatatattaccaagggactcctcatcgataagctcagattcacggtagagcgggtggctacaacaggtgtttgtccacacaagagggaatgttacctttgttgcagaacgttgctgtacagatgaacagatagaatatcatctcttgacacttatgttatcgaatagatgtgcataatagaaacaaactgggcttcaatataactagacacacacaaggagttttagtgaaagagaagaaacatagaaaacaagaaaagatgtagacttggtcatgatgtatatgttgattaaaaggacaaacagagactaatgtgcttaagcaaattatggccgataaaaaagaaaaacaaccctgcatacagaaaaatagaactatgacacacttattcaatttatgccctcctcataggatataaaggctgcataggtaatataggccggatcttgactctgttcttcaattgacacttatatttgctgtataagcacctaggagggtagttcgaaaagtcaggtctgtagtttgctttgctcccgcaagatctaacgtttggatacttaaatagaaaagcctayagtttgatgatcctcaaatattgcaatcaatacctgaagaagcaactcatattttgagttgaacaagaacacactgaaagccctgtgtagcaaattatccttttcaatcttttccatcaagtgacctacaagakacaaaagctttagcaaaccacggaggtctagctgatccaacagaatcaacatgatctattaataaaatatatggtgtcagagagagaggggggrraaaatstagaatgcccaaaacatgaataaacagttgatttccataacataaaggtatccaaaatcaaaagagtgtataagatttactgatttaggaaggatagaaagacttggagtatgcaaagtgccayaaaaccgtgaaataatacccaaaaggaagtgttgtgcagatcaaattttacttacagttatattttgtatcatgaccaacaactcggtcattctcatccactagaatgcatctgcagaaagttcacacaaagaaacctaatcaactccagacaagcgaaaaatctagataaacagcccaaacgataaatatgtagatctaaaacccaaatttgcatttggattttcaatgttttccaattatagaaccaaatatttggctcaaaaaatatctcaactcatttgataatagttaataatctatttaagtctcacctcagccgctgaacattcatttcttacaaaaamaacaaacaacaaagaaatgaaatccgaggtttaattttatcaacctttgaactagcaaacagagcaagagaagggaaaacaaagccaataattcactttgttgtgttcaaaaacaaawcaagcataatacatatccgtcgatataagatttgaagtyaatatcttcatgctttccaacaacaccaatagttcagttcatttggttgaaatgaaaaggtattgaaggggcrctgttaatagaaccaaagacaaaattaagcaaacaaaacatgaaataaatcatgctaattacgcaagatttaggaagaaaaaaaarttatgctccttcaacaatacacacacacatataatatatatatayatawatatttcgtttccttaaagaatcaaaaagagggagaacatcaacaaacaaagtataatggttgaaatataacaataaagaacaaaatacaaaccaacccawgaaataagcaacccaaaaaatcaagaaaaacaaaatcaacatttgaatatatatatatttcctattcaaaatctaagcaaccaaacaaagcatatcagaagagtaacaaagaagaaatggaagaagggttatcagataaacagaaacgcactcatcatcaaacataaggcgtctctggacagcgtccattccagcgtcggcagagtctcccatggtggaagaaagagagaccctagcggtgagaggagacaagggcttaagaagggaggggattcggagaaaagggctacgagcaaaggtagaggaagaggtaaagggaagagttctgggggcaatgtagagcttggcagtggtatttagaaggagggtcattgtcgcatgtgatctccgccattgttgaatcgagtaatattataattatatattaatattttaatgtaactattaaatatgtgcctaatattatttaatt

SEQ ID NO: 208 - DNA, 1279 bp, Cannabis sativa
gagcagcagcactaattattrttgyactaataaaatattaaatgatatcaattttgcctataagcaatataattagyyaaagcaattaaaggagcagcytttatgggatcaraaccagaaagatgytgtttrgcttctgtgttcaattgytcagcaaattctcttgatttstcaatrcccattagccttggataagtaaccttatcagccaccaaatctttcccagcagttttamccaaytcttgagaagatttrgtcacatcaagaatgtcatcaacaacctgamaaagcaarccaatacacctagcaaagctcctaagtttttcaacttcttcatcygtaccaccaccaagaatwgcccmcaaaacaacagaagcttctagaagagccccagtcttatggagatgaatgaactcaagttgttcaagcccaacattakccawaccctcwgaatcaatatcaaccacytgaccagccactaatccttctgacccaattgacttagcaagctcaccaatcgccctkacaatctyggctgccggaacaccaacggtrgagaccgccatgtgctcaaaagcaaaggctaaaagtgcatcgccggctaaaacggccacgtcttctccgaakactttrtggtttgtgggctttccmcggcgsagrtcatcrtygtccatrcaaggsagrtcrtcrtggatwagagacatggtrtggatcatttcgacggcgcaggcagccggcatggctacggattcttykccgcckacgagttcacaggctgagagacagagcatgggtctractcgttttcctccggcgaggagtgagtaacgcatggactcgtgtatcataatgggatctctgagcaaaacggctgagtctaaagcttggttgactgagttggctttttggaccatgtaagacttgaaattgawgatggatttttcttcatygccttctttaacggcttcttgctcggttagaacagcwgagattgatgaaaatggtgaagtgggtcgtttwtgttttggggtttggattgaaaagggtryttttytcaaagggtggtagagattgarattgaaagttgataaggatctggatctacctccttggttgaacatggaacaggtttgaacccatgtgagattkacagtrctcatcttcttagtcttgrcaaagattgaagytttttrattttgtkaaaatgaacaarrcaaagacactctgtgctaayrgatttaaaagcttgtaaaggttgaatttttaaatragaatctat

SEQ ID NO: 209 - DNA, 1059 bp, Cannabis sativa
cataattaaatacatttttttaatttcacaacaaaacgtcwtaatttctatagacgraaaagaaaggttgaaaatggctctaagtttctcccatcgaaatattttagttcctggcgagccaaattggttractcctcgatgattctrtctgcttcttycttttcttctsygctgctcattataaatctattggcatatccttgaatcatgcccacataaagaccaaaagttctcaacttctcaacmtcttcttcatgygcctctccaagaatggccccacatgcagcagcgcatgcatgcatcgcaccttccttcttcaaagagggataatggccccacccgacagttttggcttcatgactatcaacgtcaaatctactggctagcttctcatggtattgagcatcaatagttcctcgtgatccaaaggtgcgtgtgaactctacaatgacccgcaaaayccgwtctgatttattatgggtaaggtcatcagaaytggccmacaattcgaacccaaaaggtryaattgcgtccgggagaaggagctgaakatttgggttgtaagaattgtgggtcgctgcctcaggacttgttggattgggcctgcccgttaaagggaggtggtcatgagtgamgatggmtgcgtggatgacgcgcaaggcggaggctgctsscatggcctggycctggyggcccccgacaagctcacacgcmgccacgcacaatgacggtgctaaattcggaggtgtggaaaagatgawattgtacatggyctcgtgaactgagmgtggyggcktgatagtgatggattgcttkagatgggcatctacatcggctgttgtggakgcaaaataagaatgatgatgctgtttgkacatgsttattctcatrggtacgtgacwtaacgacttattggatttacatgtgaaatgtggaacgtaaacatgatgaacaaatcttggactgcaattaattgatagattataataaascgccattgatgaagctctagagctcccctaaaaaaaattwtataaataaagaaagtaaggaaagcaa

SEQ ID NO: 210 - DNA, 1075 bp, Cannabis sativa
catacaaaaatgggaactgaaggtggccaayatagtagagttgtgagagcaccacacaccaaaccmccattcacactaagccaaatcaagaaatccattccgccccattgcttcaaccgctctcttctccgttccttctcttatctccttcgagacctttttttcrcctytttattctactacgtagcaacytcttacttmcaccttctyccasacccacttttgtacatggcttggccactttaytggatctcccaaggctgcatttgyttcggtrtttggatcattgctcaygagtgcggtcaccatgctttwagtgaccaccmatgggtggatgacactcttggctttatcttccactctgctcttctygtcccatacttctcrtggaagtatagtcaccgtcgccaccattccaacacyggctctattgagmgcgatgaagtgattgttccaaagagraaatcacaaatgccatggcattacaaatacctcaacaattcattagggagattcttaaggcttggtcttaccgtgattttsggttggcctttgtatgtgtgtttcaatgcattaggtagaccatatgatcgtttygcttgtcattttgatccttactctccaatctactcaaaaagcgaaaggyttcatatamtartttcagatattggtgttttaattacyatatttktattgtaccaactcagctcrgttaaaggrttgagttgggttgtgatcacgtacgggatgccattactastagtaaatagcatccttgcggtgatcacatacttraatcayactcaccttgcattgccacattatgactcgtcggaatgggattggtttaggggtgctttgtcaacggttgaycgagatttcggagttctsaatggggtttttcataacatcacaaacactcatgtggtgcaccatttattctcaacaatgccacattacaatgcagtggaagcaaccaaagytgtgaagccaatattgggagagtattattgttttgatgacactccggtaattaaagcyatgtggagagaggttaaggagtgtgtctatgttg

SEQ ID NO: 211 - DNA, 1084 bp, Cannabis sativa
agctttaattattggagtrtcatcaaaagagtagtaytctcccartrwggrtttgatarctytggtggcttcatttgcatgrtartgaggtatwgttgsgaataaakggtgtgcyayatgagcrtctgtaaggtgatrgaaaacyctattgagaattgakccatagtttcgatcarytgttgacaatgctcctttcaacmaatcccactcygacgagtcataatgaggcaatgaagrgtgagtatgattcargtatgtgattatsacaatgatrctattccctataaayaatggcatcccatacaygaacataatccaactcaaccctttggccgagccaagctggtataacatcaatgcagtgatgaaaatactaatatctgacatcaatatatgaagcctttcactttttgagtagagtggagagtaaggatcataatgacatgcrgaacgatcrtatgatttgcctgtaagattkaaacctaagtacaaaggaaaaccaaggaacacyataatagaaagctgyakggctcttcctartggattgtccaagtagtgtttgtagagccatgctatttgagatttwgggactggaacaaacarttgatcatgttcaagggagccgatgttkgaatgatggcgacgatgactatacttamaagagaagtatgggaagagaagagcggagtggaggacaaagccaacrgtgtcatccacccattggtggtcactgaargcttggtgrccacaatcatgacyaaggacccaaataccagcaaaaatgctgccttggaagatccaataargtggcsaagctaagtattggagygggtrtgggagaagwtrgaartaagaggttgctatgtagtagaacaamaagrmraaaaaaaggtcttgaaggacatrrgagraggaacgaaraagagaayggttgaagcaatggggtggaatggctttcttgagttggcttaktgtgaatrgtggtrtggtgtgtggtgctcgstcaagtctacyattttygccaccggctcccatttttgtttcttcaaaaagattacaaagaaaaayaataatgcaatattag

SEQ ID NO: 212 - DNA, 1118 bp, Cannabis sativa
ctkrttcaacgtaaagacaytcytkggcmtctygccacatagccttaattattgaagtctcatywaaacratagtactctyctaatattggcttcacarcttktgttgcttctattgcattrtaatgtggtattttggggawtaaatgrtgcaacacatgagtgtttgaaacgttatgaaaaayactattgagaactccaaaatctcgatcaacggttgacaaagcacctctcaaccaatcccattcggacgagtcatagtgtggcartgsagggtgagtgtgytgtaaataagtggtcaaaactagcaagrcacctactaytagcaatggyatcccatrcacgaayamaacccaactcaatcctttrgytaagmcaagtyggtrtarmacmartgtggtgatraaaacaccaatatctgamawcaawayytgaagcctttcryttttkgagtatattggmgagtawggatyrtaatgacaagcgaaacgrtyatatgrtctwcctratawattrmaagctaagtayarrggcsaaccaatggttarwaakrmamcaagysttakgactctccctrgtggattgttcaagtatttagagwaccatggcatttgagatttcgtctttggaacaaaggcttcatcgcggtcaatggagccagtgtttgaatggtgrcgtcsrtgactatacttaaacgaaaagtatgggagaagaaaarcaragtgrataayraaaccaaccatgtcatcaacccatttgtagtcactaaaggcatggtgaccacactcrtgaccaatgagccctaarccgaayaaawtgcagccttgrgagacccaataaagtggccaagcwaagtataagagtggttgagggaaaakgtggtmgtaagaagttgctacgtagtagaayaaagaggcraaaaaaaggtctcgaaggasataagagaaggatyggagaagagaacrgttgaagcaatggggtggaatgrctttcttgagttgrcttagtgtgaatgatggcgtkgtgtgtgktgctcgatcaagtcyactrttttcaccattgactcccattttygtttcttcaawaagattarraaaaaacaaataayactaycaattt

SEQ ID NO: 213 - DNA, 1122 bp, Cannabis sativa
ttaacattgtattatttrttctctttttaatatwttttaagaaatgaaaatgggaggcaatggcgaaaatagtagacttgagcgagcaccacacaccgcrccaccattcaccctaagccaactcaagaaagcsattccaccccattgcttcaaccgctctcttctccactccttctcttatcttcttcaagatcttttcttcgcrtctttgttctactmcgtagcaacctyttacttyyacctccctcacccgctcccatacttagcttggccaytttattggatcttccaagrcatcattttttgyggtgtttgggtccttggtyatgactgtggtcaccactctttcagtgaccaccaatgggtrratgayatggttggttttgtcatycactcygcgatyctcttyycatatttttctttcaagtatagtcaccstcgccatcattcaaatactggctccattgaacatgatcaaatgtttgttccaatccccaaatctcaaaataacatggctctacaaatactacttgggcaatccactagggakagtcctaaaactttctattatattkatcmttggttttcctttgtacttaggttttaatctrrcaggtagaccwtatgatcgtttcgcttrtcattaytatccttactcttcactcwacttaaatagcgaaaggcttcatatattgatatcrgattttggwsttttaatcaycacattggtgttatacmaacttggctccaccaaagggttgagttgggttgtgttcatgtatgggatgccattgttgatarrgwatarcatccttrtagtgatygcatacttgaatcatactcaccttgcattgcctcactatgactcgtccragtgggattrrttgaaaggagtattgtcaackgttgaccgagaytatggatcacttctcaatagggtttttcatcaccttacagatgytcatgtggyrcacyatttattcgcaacaatacckcattaccatgtaaatgaagcyaccaaagctatcaagccyatattgggagagtactactattttgatgacactccaataattaaagctmtgtggagagaggmtaaggagtgtgcttatgttgagccaa

SEQ ID NO: 214 - DNA, 4158 bp, Cannabis sativa
taattttacaataattaatgaattaattataatwaattaacatgcagatccattatctgagtcaaggattagtgatttttatgtaccaagagatgaagaatttgcagaagtgaagcaaagtaattttagtttgaagactgtatactcagtaatacatgcagtgattcccatactcagacaagtcttaattgatgaaaatttcccatacttcactgccattgatgttctctaygatgaaggcattaaaattccttctaatgctgaaaagascttaatycaaaccatcaaaaatgtcaatgcaagaatatacaaaactgtttctgatgctgatgattttttacagtttcagcagcctccaaccatggacagtaagcactaktataaccttcastagtayttaataatttcggttyatrtygtttttwtratgwaaawwtatttttattaaccatyaaattycgttaccaaayaaggtaacaacttmttttgrgtttatagagktatttttagtattttcggttcgtaagttctaactttyataaaattttcataaaataattrtgaattgtcacaaattttcataaaattaattwtgagsaakttaggatattaaaatcaakattaaaataatatgtyaataagctagttraattttaattttagtatattcttaaattatccgaaatttttaagttcttatcattataaatagtccacttacaaaagaaaaaaaatatattattacaatttcttgtgtttttttcctatgaatttgttataatttttattttattttttcccttttggttatgtatatatatatatatatatttagaggacaaattcttctggtttagagatgaagagttttgtagacaaactattgccggtctcaacccttgctgcattgaattggttaaggtatatatatgctttttcatatacatatatttttrtgagsatattrstagtatttttacatgtkttattgcaattaagttaacaatactttttaagagtkattatattaaattgartattgttattaaktayyaatagtgtttastaytttttayaastarcatcytayratttattagcgatatttyytaaaawttattttmttaacttrttatatgaaamtcaatacttamttaatayaccarcgakgaggatacaargyaccaatartattatatgagactcaatamttarttacastaattawaatattaacatgtaacatyaytgattcttttttgaaatttttttaggrgataaatatagaatattttaagttgttcttattggtatatatgttatccaaaatccatgcrgragtggcctttgaaaagtgaacttgaccccacaatctatggcccaccagastcaaaaatcaccacagaattggttgagaaattcatcaaagtatatggctacaataatattaatkaggtacatatatayatacaaattcaatcayattaattaattraaaacaatcatgtagtccaaaaaattaatgcaataatacaaaatgraawttttataytaatgttatgtttattttatttatgcaggctttaaaagaaaaaarrttgttcatgttggattaccatgatgtattattaccatatgttagyaaagtaagggaaytggaaaataraacyttgtatggatcaagaacactkttyttcttgactccttatggtacattgttgyctttggccattgaattgactcggccaccratggatggtaagccgcaatggaaggaagyytmcaccccgatgaattggcattctaccgatctttggctttggagactcgcraaagctcatgtccttgctcatgattccggtgttcatcaactcgttagtcactggtatytaccattacwaataaarttatattttaytttyactmataattaaamrtacttgtaarywaaaartatttagttacaaattatcactaacatataattttaatattkcarcaaamaakamcwwatarttttagtagcaggaycaaccctkagatttattggaccttaagtatgaacaaarttakttgggcccttataaaaaatgyggtattttaaaaactactaaaaaaatwtaggtatttttaaaatatyttyttttggtctttggggctttgaactgggtggaccctaagtgtargtaggcycgacyctaggcataagygggctargcytgtrcytagggcccacctarytcarrggcccaaaaaaattctycttttaaaattacacwttttttttactaattrtaaaaaatgccacatcttttttctaaataatgacccaaaaatatttwctttactataacccactgaawtttagggccagccttaagctaagcctaayctttcttaacccttagaagtcacaatatttgtgkgmtataaattttagttaaamaaactttgacaaataaataaatmtatatwwttwtactacawgtgatatgytatgtattawctataactttttctattttaaygtggaaaattggtgcatgcatgcaggctaagaacacattgtgcagttgagccrtayataattgcaacaaatagacaattgagtgcaatgcatcctatccatagattattgaagccacattttagatacacaatggagattaatgctcttgctcgagaaagtttgatcaatgcaggtggtatcatcgaaacagcatttgcaccyggaaaatattctatggagttaagctccgtyatstaygacaaacaatggmgattcgatctacaagcattgccagctgacctaattcataggtatacatttgyttaattataacatrtaaaacacraagataaytatatttygaacawtgctatttagtatwttawggtatctaataaagatttctaatataaaataagatatstgggacytggatatatatgtcacatcatcatgttatktagttwtaayatagatagactttttgtramtaaatatyatttttagtcrcwayraaaatwagtacttattactaagaaagtagtatttarattgttactmatttaattttaatcacarcaaatrttrkggttaaawacacatttastaryaawaaattrtgatttycstgattaatatttttagttatggaytttttattagtaataatatargaatgatgatttatgtttagttayaattatttyacttttaaktamaaawtwtrttgcaactaaaagtaarattattttgtwgtgratgttgaaacttaattctatactatatacttttcatttatatgatctcatcataattagcttgwaattcatgyagrggaatggctgttgaggacaagratagtgarcatggtgtaagagtaataattgaagaytacccwtacgccaacgacggtcttctcatatggagctccatcaaacaatgggttactgactacrtcaaccactactaccctayctcaagtgaggtagagcgtgaygaagaattacaagcatggtggacagagatcagaactgtaggtcacgctgacaagaaagacgcacctgggtggcctgacttaaaaacgaaacaagatctcatagacattgtcacaaacatggcatggacwgcrtcagctcaccatgcagctgtcaactttggacaatatgcttacgctggctatttccctaaccgaccaacyataacaagaactgttatgccgtcagaagagaaggagtataacctagatgcgtggaaacacttyaaaaatagtcctgaagaygcccttttgaagtgcttacctacgcaattacargcaggcctagttgtggcygtgttagacgtsttgtctactcactcgccagacgaagagtatcttggagacaagatggaaccctcgtggggctcgaatcttrttatagcggaagcttttaatcggttcaataagaggatgaacgagattgaaagtatcattaatgaaaagaatgataatgagaatttaaggaatagacatggagctggaattttgtcttatgaacttctcaagcccttttctgagcctggtgtcactaacaagggtattccatatagtatatctatttgaaataaattttaaacattccatatagcatatctatttgaaataaatttta

SEQ ID NO: 215 - DNA, 7199 bp, Cannabis sativa
catacatgtgagtcagtcacastactcaaacaaaagcatcttaaaaccatgtcttttatgatgagcatraatccttctccctcctcgccacckccaccgttatcgtcgccktcggaatcttcctcaacgccgtcaacactgccagtccgtacgatcccgggaagctacggatggccgttactggggcccatctcggaccggttagactacttctggttccaaggcccagatacgtttttcagaaaaagagtrgagaaatwcaagagcacmgtgttccgtaccaacatacccccgacctttcctttcttcagcrttaatccgaacattgtggcsgtgctggactgtaaatcattttctcatcttttcgacatggarattgtcgagaaaaagaatgttcttgttggagayttcatgcccagtgtcaattacactggkgatattagggttggagcttatctcgayacttctgaaccacaacacgctaaggtaattaatttcctctcaaaarttatatttaacaaattaattaaaatcyttgattgcttattctttattgtgttaattttggatkatttgaactrggtttttatatattattaagatagtctttaataawytcttgcaacatataattttaagaagacagaaagagagagtgagatgkatataataatgtctagaaaaaaaaaaaggactaggattatataataagatatttagatatttaaaaaacaaatctgagattgattyttatttattatttkgaaaggagcaaatctgagattgattgaataattgggatcagagccaatggagtttcttaacactaagttatgattcacattttttttattatattattatttttttgggatagsgatttacatgtttattaaattaattgtggtgtttttttttttttttttygaaatcaattaattgtggtgttagttcagagttctcacttctcttacatattaataaatggcttaatataagtcaaaataaataaatggcttawtaaattttacataagaccgaggttagattttttttttctttgaatataaaggaaaagactaaaaggcccaaatgtgtaacacgattggtgtactattccaatyaaatgaacaagtggacgatcttggtcacacgtamgggcaaatgcaaatgcaatcaratrgctccattcgaaatttcgtatgtatgagattctgaggggagaattacttttgccacattattttgggaccattctcttatactaaacaccaattgtccttttcctatcakatggctctcctaagaaccaatgaarttatgtgacttttttgktttttttttttttttkaaacaatgacttttttggtttawkaaagrgtaaatactatttrgacattatrttttgtaawagttattagatgaaccatctgttttgttaaatgayaaaataraycctatattttcyaamatggtaaaaatargacmttcarctcaattttcaacaattttatttttyaatacaastaactttaagaaaatttctaacacgaacarattcagaaaatataaycagttttgtyataacatctmtagatcakattatkattaagttttattttgrtaaaaaaaatyagtkcawggtcytatttktacmattttrgaaaatatargrtytattttrtcattkaacaaaayaatttgttcaattrrtmaattttgcaaaacacargwtccaaaatagtatytacyytttaawaaatatatakcctagaactaakactagcarcacatawatataggtatctagcattgttttagaaaataatattacgaaaacatagaggcttttaagttgtattttcagttaaaagtaagaatagaaaaaaaaaattgtyattttaaaaaawttaatggtrtttkrctcaagttttttaaaactatttttaattttttttytmwaaaaaaaaaaatcaataccttacaccatactatgayatgaactcatcctrcctcaaaaayaaaattataamtaaagagtcgtttgaaaacctctacataaaaatggggttttgaaatctctygatctctgatatacttctcaggrtattygagtcgttgattykgtcatttgaakacttgtcctctrccgtttcgagttttctctttggcttcgttctctycgttctcgccttgcatmtgtattctcttcrcctwcttcgtgccctrtcasgtcyggtcccctattygacgrtgcccyagtagagagatgccttgcaratktgaacttttkaggctyccttcaratgacrgtggagagtttggtgcccgtcgawgctctgtttgtggccttrgttttgagcgrtcttgaaggtattrgtttctawygggtrktggctggagcattaggacatgcctctcatttcctytggggttgggtttyttgttttagtgagagcttttctwcctcaaagcctawtgcagtyarggtctcttggtatrtgmtttcttgatttctttttgytttttgtttagattatttagtgttgttgctttagggtttctatttttagggtttgaggwtgtgatgtttgagtrggatctctgttcatctrgttgtgggtttyggtttragatttacatttttctcgayaattgagaggtttttgtgattttrgattgggktgytgtggacttytttggmctgagagtktctgtgattttactttttgtcrctcccytggttgytgagytgtttattgcatattctgggagggagtracatttggttagaagtttggttccataacattaatgataattgggtytttgctaccttygtggaggttttggttyaagktggggttgtcgaacatcacccactggtttcatgaaaggggttgcttgcaattataagaactytaattcgaggttctaraagaatgtggttaaggctaaggatgttctaawgaagggtgcttgctmtgtgatagctgatgggaaagatatcaacatttgggaggatctgtgggtgrcccatggtatcgatttatgccccattscraatggggtttcagcccycarctttatgactgtggcaamtttattactagaaaatggggagtaggatctttgtaaactaaataggttgtatgatcatgacacaatgagtgccactttggtaagggaaaggccactaggacatgggagggatcgctggatttgaattaaagaattgaatagaaaattctccaccaaatctgcttaccttttgcaggctcttgagagagaccaacattgtcaggtcgctccttctctgtggaataaattttggagcaacaaaacacttgaaygacataaagtgctttggtggagcattytatccaatgcacttccaattcgatcccttcttatagatcsaatgaacattaaagactcctcttrcccgatttgtgggagagaagaagaaactatggaacatctttttcttcattgtaacatggcttcwcatctctagagagtcgctccttggrgtatcattcmtgttcaggaaccagaaatcaggatatgagacyatgtcaaattyatttggaggttgytagtgagagatgtgaagattgatgatgtatttttatatgygtctatactagttgacactatttgragagcgataaaggtacataawaascatctaaataacataaatttttatagtgactctattgctcattgttatacaaagtattgccaatctcttttggatcaatcttaactttcccatgmtatcactgagtgggctcctctgcttgyggactggatcaaacttaactgtgatgtgggattagggaggggcctatgatgcttgcggtkgtggctcmagatcatgttgggaagatactytaggtcggtactaagcgystgccttttgccaatacttttattgggaaaattatttcttgtctgctggcrttrgaacttgctgcaragaaaggtttagaatttgttattgtgaaaagtaattctaacacagtyattaaagcagkcaatggtttgaacccgaggtaraaaattgatarctatttgactcattgtagaagccttatgagctccttctyaagatgtgcttttttaaaggtttctagatcttttaacttaatttgaagcccacaatttggcktaagtrggcgtttagtmaaaatgtaactggtagttttgaaacatcctcaataccatataatattgtttgtartggccattgggcctaattwgatttcatttatwaattacactttttttgaaaaaaaaatgaacccatcctaccttgggccgaaccttgacatgaacytggacytcgtcccaaatccaaacccggacccagacyccagacctggggctaggtctgaggtcctawgctaggtttgtggtctgggtctggaacctgtccggggtacgrggtcttaggtctggggttcgaggtctgttgtttggggtccaggttcggsgtttgggatctaggtttatgttyggtctagtgtccggtgtctggggtctggggtccaagtttagrtctagrttcgggttcggggctaggatttgggtyggggtccaygtttgggttcrgggtctgggtttgtataayaaccaacaaaactacttaaattgcaactaaaattcacacaacaacccatataaactatcggagcaacatcaattgtaaatttttaatatatatatatatatatatrtatatatrgaataattcyccaaaaaattatcttaaactttaattatccctcttggattgtggattaaaatatcccaagttgttttttttttcttaccattaattagatttaaaatatatatgtgcattacatatattttaataattaaaatttttaaaaataatgaaattttaatctataaaataataaagtagttcaattttaaaagtttattaatatattttcttttttatttgttatgtttattaatatgaattttaaattatagaataataaattatatattataattttgtacatattatttaatttctagagctattaaatttattgaaaattatttattttatttatttttatttttttattttattttattacatatttatttcacattattctttagtgacaaaaaaaattaaaaaaaataataaatttattaaaaaaaaaaaactgtatagtcctgttctaagccaaacataatacaaatatattttcttttttctcaargtaaacatgatataatacttaarttatccaatatccagtccaatcatrcattackttatatascaaaagagccctaaggtattcactacaagcaacatgtttttgaggggccatgatatatgaaagcataataagcatataattttgcaggtacattaagtttagtggttcattkaaatatcaatytaaaytttagaattagatsgcagaktgttytggttgaattgagtcatattataatgtcttamtaatgctattttawatagagatgcttataaatgaagtggttgaaatcaatgaagtacaggttaagaacttcgcaatggatgtaytaaaacaaagctcgaagatatgggtgggagaactgacatcaaatctgtcgacgatgtgggacacaatagaaaaagacgtatctgagaaatcatcctcatcctacttagccccacttcaaaagttcttgttcaacttcctggtcaagtgtctaattggtgctgacccttccaactcccccaagattgcagagtctggctacatcatgctcgaccgatggttagccttccagctccttcccactatcaagattgggatccttcagcctcttgaggagcttttcattcaytcttttgcctatccttttttcttggtyagtggtgactacaataacctctccagttttgtagaggaatatggttagttaattatrctaattacyatctctactctcyacacaaatatttatwtagtgctgyattgcattgttttttttagtgtctaacaccccaccttatgttgaacaatcatggtcatataaagctgcattaaytggtattagrcactattaatttagagatatgtargaagtratatattgttattartacaccattctctccaaaccatacakacaagtgcacgacgagcccatgcaaagkgtttggttaagatatgccagattccaccaaatatacaaatggaacctaatcatacatgtcctctaaytatatcttccaaatcrttcacactaacaatycacctctctccaccaaargaagatttgagtaaataaccaaatatggtggtgcggtgatagtgttgtgttagactcatgaccccattaatgcattggcaaaatgawtttttttgtaaaatttattttgcatagacaccccttgtcattttgcaaataatttccacttgggagtaataatacactaaaaattaatagtttgagaaattatttgtggttaccttttacaggtaaagaaatagtagcgagaggtgaaaccgagttcgggctgagtaaacaagaagcgattcacaaccttctcttcattttgggyttcaacgccttcgggggattctctatatttctaccgagcctactgggcaccgtggcgagtgacacaaccgatctacaacaaagactggtcaaagaagtcagacaaaatggcgggtcaactctgacgtttgaytcgatcaaagaaatgccactcgttcaatcggtcgtgtacgagactctccggctcaatccacctgttccgctccaattcgccagggccaggaaggacttccggctcagctcgcacgacgcggccttcgaggtgaagaaaggcgagctcctatgcgggtttcaaagccttgttatgagggacccaaaaatattctcggaaccggagtcgttcatyggggaccggttcatgaaagataaaggtctsttagattatctttactggtccaatggacctcaaaccggtgtgcccagcgtsrccaataagcaatgcgcgggaaaagatatcgtcacgcttwcggcttgtttgatcttggcttacaccttccgtcgttatgactccatcagcgggagctcaagttcaatcacagccmttaaaaaggcttaaactactgaatcagaggagtatggtgatggtgatggtaatgacraactta

SEQ ID NO: 216 - DNA, 6687 bp, Cannabis sativa
atgaacaccwaaatmattaatrgttgtttttcttttattatgattcagggagaaagtaaagtccacyaatmtgggcgcacttttggagaagcgaggaaaagagtttttgggagtcaagtataaggatcccatttcaagcttttctcatytccaagaattttctgtaagaaaccctgaggtaatamtaataacyattwawkattttwtktgagggtrgtwtttatrttaattawttgtaattaatggytaatattatataatgttaggtgtattggagaacaktactaatggatgagatgaagataagtttttcwaaggatccagaatgtatattgcgtagagatgatgayattaataatccrggggktagtgaatggcttccaggaggttatcttaactcagyraagaattgyttgaatgtaaatagtwacaagaaattgaatgatacaatgattgtatggcgtgatgaaggaaatgatgrtttgcctctaaacaaattgacacttraccaattgcgtaaacgtgtttggtatatatmcattgaatttactttaaatctttctgtttttacatataacaagaattcatawagttaatatkatatgstttayttggataytgaaactatgtatggtaawtaatttgaaggttagttkgttatgcacttgaagaaatgggtttggagaagggttgtgcaattgcaattgatatgccaatgcatgtggatgctgtggttatctatctagctattgttcttgckggatatgtagttgtttctattgctgatagtttttctgctcctgaaatatcaacaagacttcgamtatcaaaagcaaaagccatttttacacaggtatatacatataatatatggagaacgttytaatagttacattaaatatgtaaccatsatggttacakttatttttagtygttgratyattattaracgggtatgattaatttaaaaaattactaaaaataactaataaattacctgcaagatactaacctgatttratttatttcttttaaataaaaaaattataaatattaatttctaaaaakgttttattaaatactttaattaaaaaataatttatttaagtctaaaaataraaatttacctatcagtgaactgttataacaggttataaaaaaaatgtaacyatyaatactcacgcataaatatatatatatatatatttactatawatactcaytgcttatatagacatatatactaacaaaaarytaygtgcgagrcacgtatactaaattttakgytagtttttttytaygttatttaaaagtgatacmattaaaaattaaaggaaaawtattattwgttattaggtgagattattattatgtrtatytaaataatatarttywtaattttgayaatkraaagtaaataycrgatrcaatatmttagtrtttaagaaagctkratwatttaaatrtgttcttmarttawyymaaaaataaaytaaaaattratrttmcaatacttaaggaaacataayttaaatgtgtgtaagataaaaaagmaaattaaaaatcaatytctaaattgtagcaaratytttttatgtttayttttctwtgyaaatttaakgtgtwagctgaatatcactgttaaaatgccaactgaatwattattgtattgacttcgattttactttcacstatacacatgtgtaaacrgtrttcatccarctrrtatttaatkgtgatatttaacygacgcatcaaatttacaaagaaaagtaaatttaaaacataarrrrrttttrcyacaaaaaaatataagrrrttaagtgtaaaaaaattaaaacrtaaarmatttyacygacmattacytcatatgtatatatatrtgkatattttattttagatttattaatttaaaraattatatctatgtctatgttatattaaatgyagatayatgacaaaagtkttattttttttttctaaaaaaaattacratttyaaatttaamctcaatatacatatctctcttatytttkaccttatattaaatgtgtctatataagatattttttttttkatattgcaatataaattaaacacattgcatatatttttttttttttaaaaaaaaaattattattatttttttttaccaaaaaaatatattttattattattatttaaaaaaattaaaaaaaktgtataaaatttcaaaaacaatgaaaagtattataacatyaaaagtattacactaattaaattaaaattawaattaataataaataacttgtaaattaaatatttgccccccacttgcaaattctctcycggccactgacgataaagtatacttctccwaattttatgccaatagctagtaatttagtttwaatttgctttttccctttgcyaagatasctagtaatgttgttgtgwtatcaatttstacaatatataatgtcatttctctttatattatttagtaagaaagtaatatatatacatatatatatcctattttagytwttttttttacttaattaatttccttcgttttcaggatcatattattcgtgggaagaagcgtattcccttatacaggtcacattctctttatccgattttaaatttttaattacatttatsaattagttaawtaaaaaatgacatttaaatatcattaattagwacacattcaaaaatttatyattcctgacaaataataataaaattaytattattattattcattcatccatctcacaaaataagttaaattgygcagtagagttgtggaagccaagtctcccatggccattgttattcctkgtagtggctctaatattgstgcagaattgcgtgatggcgatatttcttgggattactttctagaaagagcaaaagagtttaagtaagttaatttattttasttctttttgttacatcaatttcaattgtttaattcrtagarattaatgtggaatatataaayagtagaggtgaatatayatayatatmtatayatatatgaacatttgratccattggagtttgaactaaaayamatgtgaatgktagtggaaaataagtatcacgtttcttattaatcaaggggtttwaatcaagttgggyattattaatttaggagttattttggggcttaactaaattcatcttatttttttatyttattaatktattttttttaaytaaaagtaacacaggcaaattagttmtatgckgacaaaaagaaytatatgattatttaagtattatatctagaatattattaaaaaaragaaaaaaaaagtattrtctycaaaatartaagcatatatgtgggactaatatcttcytgagtggyacstgcagaaattgtgaatttactgctagagaacaaccmgttgatgccyatacaaacatcctcttctcatctggaacaacaggtaattgaagccwccrccatgttttgaatttatwaaaasarcattkggaartaactgatattacatatttktatgtagayytaatgatcaasaatttaggymttatatatcyyctgtttaagacaataaragagtytatactgayaaaattaragaaaaattacarttttamaaayattkrgtacygctaaaaataaacawtrrrtttatgycgcttacaamyrtaaaactttgastacttatrsygctrttyatycttaaacaaaacatgyctacaactaaacttktaagagcgcaaactaaaaggtaattcagtwaataatgatgsttggtatgaaaaagaacacattctcaaaawyaaagaccgttaaaaaatctcaygagcccccttaatwtccttaattagttttgttcatcaatctaaacacttttgatggaagacaatrttgttttggtataggggagccaaaggcaattccatggacycaagcaactcctttaaaagcagctgcagatgggtggagccatttgsacattaggaaaggtgatgtcattgtttggcccactaatcttggytggatgrtgggtccttggctggtctatgcttcactccttaatggggcttctmttgccttgtataatggatcaccactwgtttctrgcttygccaaatttgtgcaggtaaactaaacttccattaactaataaactttttarttatgttatttatgtgytttgtgsttttctatakataagttatatttgtaaatgtctaattmatyatagaagggtgaaaacagaaagtacaccaataatcacawcatttttattattattattwtttaaaataaaaaacatgataagttyggtatttagctaaaacatwttgactayaattttattttcmctttatyttcyctttattttaatgtttttmtaatatgtgygtaggatgctaaagtaacaatgctaggtgtggtccctagyatwgttcgatcatggaaaagtaccaattktgttagtggctatgattggtccaccatccggtaaagaattataatatattttccaatawttcttatstatgcacataacatcartstagattatattaaacgtattatatggatagttaaaataagttttatatcccttcttgtagttgcttttcctcttctggtgaagcatctaatgtagatgaatacctatggttgatggggagagcaaactacaagcctgttatcgaaatgtgtggtggcacagaaattggtggtgcattttctgctggctctttcttacarsctcaatcattatcttcatttagttcacaatgtatgggttgcactttatacatacttgacaasaatggttatccaatggtaattaataaacttataaaatataatwtamaaatattggataagacttttcaaataagatttgatggatcaatttaaattcacaattcttgtattaatcccatataaaattatgttacatttgactkrwttgttgtattatatatatactttatgacatgcagccwaaaaacaaaccaggaattrgtgaattagcgctyggtccagtcatgtttggagcatcgaagactctgttgaatggtaatcaccatgatgtttattttaagggaatgcctacattgaatggagaggttttaaggaggcatggggacatttttgarcttacatctaatggttattatcatgcacatggtcrtgcagatgatacaatgaatattggaggcatcaaggtatgaaatgaattatgtgtgctattaattagttaggttgccctgccyamcaataatttwwtttttatttattttaaattwactattttgatcatatattttwttttttataattcttaattaggttttagataatsacacatattcttagttcatagacaaagtaggaaattatyctggttwagaacctaagttcgatgtaaacttagtcctacttcagcttrgaatacgatctcagatttckttattaattagcaacaaatcragtacatcacagtygagwaaggattctcaatcttgggttcaaacaagtttttcactttcacactctcatccgagcatacaaaacgtcaatgtagctcattacaaaaaycgaatcsycaaactctttcacagtaytctctcyttatctgatcttcttgcactctctttctctagattcgtgcttctrtsgtgatctaaaaaatattcactaaataatgaataagacttgtcttatataggcaactkagtaagtagctaaattaagaaaggtcagctaaatcgatmttggataataacaaatgtarctaaatctgctatatctcttgttacttttggtaatttgatagrattrtrrctstacatatatatatttccaactatagatttgaattgaataygattttgtttatgtaaacagattagttccatagagattgaacgagtttgtaatgaagttgatgacagagttttygagacaactgctattggrgtgccacctttgggcggtggacctgagcaattagtarttttctttgtattaaaarattcaaatgatacaacwattgacttaaatcaattgaggttatctttcaacttgggtttacagaagaaactaaatcctctgttcaaggtaccaatttcatcattattaacttttttgtttgttaatttttagtctygactctcaakggtgtatatttmgtaaaaaattaractattcttwtcctatacacttccacactatgatgatgatcttataattktwtactarttctartttggtgtttggttaawtattaaatwggtcactcgtgttgtgccyctttcatcacttccgagaacagcaaccaacaagatcatgagaagggttttgcgccarcaattttctcactttgaatgaagtgttataatataatacatacatatatgctttgaataaagtgtagttgattacatagagcagtttgcattatcaaatrcactaataataagttaaatgcwctaaaatatgtgttaaatcttatggcaggattatrgatttgattaakatastcatattraaccataaattttyattcaacacatttttagwgcatttgacattatttttattgaatttaataaatactgactacaytgtatagtccactacatcatatcccttcaatataatatttgtgatttagaataaraatagttactttwaga

SEQ ID NO: 217 - DNA, 1416 bp, Cannabis sativa
atcacacatacacaactcattattatcataataataataataataatatgaatcatcttcgygctgagggtccggccwccgtyctcgccatyggcascgccaatccggagaacattttaatacaagaygagtttcccgactactactttcgggtcaccaaaagtgaacacatgactcaactcaaagaaaagtttcgaaaaatatgtatgtattatgaatatatatatttccaactaataatacttttccatgtaygtatacgcattatctatttatataataaaggaatttttacatcatatattaagcatsaagaatttatgtttaatatcttgtatcattacttwctttatatgtatatgtaggtgacaaaagtatgataaggaaacgtaactgtttcttaaatgaagaacacctaaagcaaaacccaagattggtggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagygcatcaaccactgacatgcccggtgcagactaccattgcgctaarcttctcggactsaryccctcwgtgaagcgtgtgwtgwtgtatcaacwaggytgytatggtggtggmacmgttctacgyrttgccaaggacwtagcrgagaayaacaaaggsgcwcgagttctcgyygtgtgttgtgacatrayggcttgcttrtttcgtgggccytcagattmtrwcctcgaaytactagtkggacaagctatctyyrgtgatggggcwgctgckgtyattgttggwgcygaacccgatgagtcagttggsgaaaggccgatatttgagttrgtgtcaactgggcaracawtcwtaccaaactcggaagsaaykattgggggrcatataagggaagcaggmytgatrtttgaywtacayaargatrtrccwatgttgatykctaayaatattgagaaawkyttgattgaggcattyactcctattgggattagtgattggaactcyatattttggattacwcayccaggtgggaaagcyattttggacaaagtrgaggagaagttgsatytgaagraggagaagtttgtggattcacgtyatgtgytgagtgagyatgggaayatgtytagcycaactgtyttgtttgtwatgratgagwtgagraagaggtcgttgraggaagkgaartcyacmastggrgatggatttgagtggggtgttctttttggrtttggwscaggtttgactrtcgaaasagtggtcstgcgtagtgttccsatcaawtattaataaaagatrataataaatgataaaaasaayaataaagaataatgcccaat

SEQ ID NO: 218 - DNA, 689 bp, Cannabis sativa
tcgacwgtatgtagcataatgtgatttatataaytatcaaaaaaaaaawraaaagaagaagaagaagaagttgagaaagagaatggcagtgaagcatttgattrtattgaagttcaaagaygaaatcacagaagcccaaaaggaagaatttttcaagacgtatgtgaatcttgtgaatatcatcccagccatgaaagatgtatactggtaatttgtgattattttgtttcataattcatatacatttarctaattattataataatagtatagtsatgaataattctaataatgtwgtttgcaytgcaggggtaaagaygtgactcaaaagaataaggaagaagggtacactcacatarttgargtawtgtttgagagtgtggagactattcaggamtacattrttcatcctgcccatgttggatttggrgatgtctatcgttstttytgggaaaaacttctcatttttgactacacaccacgaaagtagactatatatatatatagtascsgaccaagctgccttcatcttcatcttctcaaataatatatctaatatctaattatataataataactacttaataaaagactgtgtttataacattaaataataataataataaagtcttttgtagctcagctcatatctcttataattttttaytttttatttttttttygattga

SEQ ID NO: 219 - DNA, 496 bp, Cannabis sativa
tcgacwgtatgtagcataatgtgatttatataaytatmaaaaaaaaaaawakaakaagaagaagaaagttgagaaagagaatggcagtkaagcatttgattrtattgaagttcaaagaygaaatcacagaagcccaaaaggargaatttttcaagacktatgtgaatcttgtgaatatcatcccagccatgaaagatgtatactggtaatttgtgattattttgtttcataattcatacatttaytaattataatwatagtatagtsatgaatataattctaataatkttgtttgcattgyaggggtaaagatgtgactcaaaagaataaggaagaagggtacactcacatagttgaggtaayrtttgagagtgtggagactattcaggamtacattrttcatcctgcccatgttggatttggrgatgtctatcgttstttytgggaaaarcttctcatttttgactacacaccacgaaagtagactatatatata

SEQ ID NO: 220 - DNA, 7381 bp, Cannabis sativa
atmattaagaaraaraaaawayatacaaatatatagaagtaagagacacacacatamcctaayggaattttttkckattgataatgattctgagcatttgttttgtagatgaaaactcttggkggagcaatgtttagagggaaaattattgtaagagtatttaattggtgttttrgggtgtcgatawmataataatgaggttttgggattattattgtgaggatttaataaagtatggtaattagtttgaaatgaaaaggtmcaaactgatgagagtcccatgaagattrttattgatwgaatattaaraaaaaatgagggaaagaaagggttagaataacatagtgatatcaataatgaaaaawtarcaagatttatagtaaaayaatttttscttggtttcaaaatttaaastmatmcatgttttagaagkcatgcatgktagrcacatctatctttcttgcctaattaaytaattatggctatmacaaaccrtgcatgcatgtagagctctacactacgatgagattaaatttatacaatcatatcaaaattaatattttgatttagtgttgamaatttatgaattattgtaactattaaaacatgttcggaaatccctcatctcgaccaccaaataccatctagaatcttcctayaggacyctcctacccgragcctcacawctagaacttccaatccrgracctcccaactagagscttccraaataaatccttaaccasacacacacacatttttgaaactatcartttggaccccawctaaaaayytttgatccmgaatataccatccaaacctcactcatcacctattcgggatcccttrataaggatactaacaaccargtcacaccwatccaagcaaccaaaactcyaggctcttaactctagattcaccaaccccaaacctaatcattgaaaacactrtctaggactacccttccgaaaccttaccacwcaaatcacayatgacaagtayaactcattcraaaccattttgggcaraaaagagttcaaaactactcaactttcaraccttcaaaattaagtcattccttgaccaaaggctatcaaacttaccaraacaagagggtaatctaaacaaaacatacacttcaaaagcayctmaaaccacctaaaactaatttcaccrtattgttgagtggactttgagttcaaaaactcgaactcctcmaacacgaactacatcagctaaaatcttacaaaccaacacagaattcaaccttaaatcaaaccaacaacaattttaasctcaaacaaactctacaaaacctaacccataaaatccaaactaattacaacaaaagcttttcttttaaaaatttaaagrgacaaaggagataggttaccttratactccttctaattcaacactaactctagttagaatttcttagttaattggtgtgtaaattattataaaaaatctrgttygargttgktatgacctaraaatrgaccaaatggatgtgaaaayaayatttttrcatgggagatcgacttgaagaataawtctamatggaagagcytgatggatacaaaascagcaaaccaaaccaggtatgcctcctacaaaaatcactttatgggcttaaacaagccccaaggcaatggaatctaaggtttcatgaatttwtkatyagtattgggtatagtagaagtrattatgayccytgtrtgtattataacaatgaaccaatttggttactattgtatgttgatgatattctcataattgktaacaaaaggcagkctayagmaaaattaaaacaaagtctgaatrcagaattcraaatkaaagatcttggagaagcswagakratacttrggattgacataagaagagacagatcagagaagctttcactatcatagaraagttacatacraaaattgatacaaattctrcatgagtgwagcaaaacyagttagcactccctggcccacatatttcaagttaacacagagcmaatcacccaaaattgaagrggmraaaatgtacatggatacaaactcatgtttgtgtatgaggtacatgatcaagcattgatcacatcakttmaartaataaattcatgtttgtgaatgatatatatgttgtgaaaggaggatacaaactccttgttgtagacttccttacaaagcatcttgtggtgttaaaaycyggcagattcttctcattttgctytcactaycgcctttttaggcatgakgattcgytgtggaatcttcatgtcaactctcgtccacctataactgtttacaagtatggtttttcttcattttaagtgagagcgaacattaatttaaayataactatgtaaagattygctactaarttatatcaataayaataaattttatttataatrkgttgagagtaagtgtgttttataataacaaggctctaaatgaaaacatatactaaatattcagcataatrragcttccacatgaactcgtaraatyttctgcctgcttcctgcattgtacaaccaatatatatatataaataattaatgtatgattaataattaattagaaaattatattattttttatgtatataataataataataataataataataataataataataatataataataataataataagtgtatataattaccgggtygtaatttgttakmgcaaamtctcgagtctgtayrcaattaaacaattaaaaatatttataaatatgtgaaaaaaattattataataatatatttgctcttaatttcttttgwgagaatttaaatggmaaacctgrarrattaaccaaaatgctaagrttgcatgwgaaagtaacattacgttactsttgaaagcctgtaattaaataagaaatgaygaaaagacaattaacattaataaataaaatacttgaattattttagaaataataataaattaatcaagaattgaatggatkaatcttaygagtatkatttcgggtgtgrctcatatggtaaawataatatgatatgaatttaaattttatctaattaaattagaatatgtattaaaatgtaatwttaaattatacmaataataattaatggcatgtgtgtyagacaatattttagtacctcataatgtataattaataaaatacctgtggccaaataatggcagcaagtatagcagmyacataggttagragaacaattccagaacaaagaaatgttatgtttttagcaccatatctggttgccaaggttgatatgccatrcctatatagaaataagataataagtaatattattaagtgtktgtttaaaaaaaaaaaaaggaaaaaaaatgataataaaatatttataaatgataattttaaataaaataaytaattaaactaaytaaggttggcataagaatttaacatacttggagtcsccytcaatgtctgaaacatctttacataaaaataaagctgatcccattgatttaacaaaggctaggatgaaagtaaaagraggactgaaactcaaattcaaattcaaattcaaacacatgatgagatagttataatayatcaaattttgataagaatcgttcagcaagaaaaaaaaaaaaaacamaaaagtcgaattatcccaatgaagaaataattaaaaatcattttacatgatatacctcattaatttaaattggcaacatttttttaattamctttttatttttytagaaaaatgaaaatrctttttaaaaayatsttttataaaattagttttmactttcatttttaattragaattaaacttttaaaaatttgaaaayaawmcaaaaaaaataatcactttaatccattttttaaayggttttttttcttaatcagtattttggtcgactttgaccccaacttagaccttggtacccaaaatycgrccctagccccagtcgtggactcggaccacaaccycgacctcaracacakayttgaattcgacttaaataaaawtaaaattaaaataaattaaaaaaaataacartggttatagagaagtacatttctattttttaaaaaaaaaaaattaaaaacacaaawtttayttttatttttrtgattaaaawwwwaaaaaaaaaymaaaagkattaccaaassttcccttaatygytaaaawttaaagtgatcattgraataattttagttagtctagartttgattartttctcrttttccatgwtttttatttttttaaacaaaagtayatgattyttcttttattttaaccttttttatttayttcaaaacaaaatctttcatatattctcaatatttaattaatacactatgatgacttttttttttctttcaaaaattatgagaaaagmaataaaaatkagacttaacaaattaattacatcgatagttgtctttgaatatttttggagtacaaatattgtaatcaaaatgatggtagtatttatttttctattttctttagttttyttttttttattgtttaattactaacaaatwtattgatatgtgtaatttttattggtattaatttgtggtagtacccacctcatctcaaatggtagrccaaaagctgctctgctagcataatracatgtgaaatttamgatcacsagaccctgcacagtgttatatattwtaataaataattacctcatattaaattgatttcttttgaaaaaattaattaattaattagtwatagaaaaattatcaaaagttacmgttaaagataagaagtagtaaacgtgcactaaaatactactttyaataacgtagcagtgttgctakaatattttgtgtaacattcattaattastcatastaaagtaaacagtaatgatatatatataaattataaaatatgctcaggccttaatatraaagtgatatttttttctatatawattatttgtgtgcatatatcattwcttataataaaataaattaccataaaataactggaaaatgcggtattaggattttgcttccatctaaarggtggaacagaatagagakctccaaccaaaatactaacacaatacagagaaataaagagtgktccacagttcaattttatagttagtatcaacccagttagggccacaattatgctcataatccaagctgtttcaattgacatttcccctgaagctagtggtagatcaggcttgtttawcctgataatgaaaattatattaattaagtgtaatcaatattagtttaattttacaaaattaaaaatgtagtgataattgtacctgtcgatgtccaaatcggtaatctgattaatgatattagyacacaaattaacgctcaaaataacyatcaaagaagwgaatgccttsaacatcagagaccaactcaataaatttgtgttatgaaacaactctctcccaaacaaaccactgttctccaataacaaaacaaaattaatatacatcaattscaatcaagttggtgcacatatatacacttgatacacatattacaaaaaatyttattttaattactacaaaatttataactaaaataaaaatatkgtgractaaatatgttagcattttattatgaataagagtgatgtttagtttcatwaaatctaaattgkgactaaaagtcatcattaattataaaggatatasgttttttttyttagtgacacatgattcatgaataaaggaaattttygctsaaattataaargagtttttacatttcatattsaaattttwaatttttatacttttttattgtrgggtaawtttttttcaaaaatamtgtttttttttttcgaaaatactgtgtaagttttatactgtgtagtgtgtastgtgttaagtttttactgttgtttcasggttawttttagtttttcactgttrttttaattattctgttttgttgttttgcgattgttcaataaaaagacattatttttataaaagaatttcatgttataataaaattataaatttttacycaaaatktaatatttttctaaaaacttctaatttttgttagaaatgtgtcccttttttcctattgrtaagagygtattcttgaatgtgatcaaattatagatcggttwtttttttttttttctataagatygkmtacattaaagttatagtagagtacatccaataggttttgaaaaaattaattatraataatttacaatattcaaaactaaactcmaaatagtaaatttaamtgcacctacctaaaaataaaaaataaaaaataaaaatgtgtaattatcsgtttgaattctstttttracaatataaattattyaaaaatattcaaaatttgataaaatatttaatataattacaatatataytggyaacttctgttattagtttaggggattatatttagamaaatgyaatataatrtrttagattataattrtwgtatttttkaaaagtgttatastattagaatgtttttttttttttttwwaaaaaaaaagtgttaaaaataatrraaaaaatataaaataaaatrttatatttgagatttttaatatttttaaaagatatgctacctatagtgatgcttatgcccctctccggattcttcctaatcctakctagttacacacatagggtaacccaattagktacaagttacaaatactttttttccctaaaattttcctaacatttggrgtaagtacaaaataataattattaggaaaaaawaacatttttaaaaagaaaatgtttatatatataattatacaaatgaccrtatttggaattaaaattaagagcatattgaaaataatgagtacttaccaagcgcaygagataactcctatgaatgtatacggtcwttgaagtttccaacatgtgtgcccraagctttaaaattttagttactactgaatatttagtattagattcaggaggctcagtttgatttgcagatayatctgtctattaartgttaacacamtattatttttcttaattgtaaaataraaaactcaattctatcttatgtataatttargaagaaagaaagaaagaaattsccatataaatatatayatctacaagcaagaaacccacacataccctaatggaattwtttgcaattaatawtgaktttgagcatctgttttgtagatgaaagttcttgttggagcaatakttagagggaaaattattawaagargatgtaattattggtgttttggggtgttgatragataatartgagctcytgggattcttattatgagggtttaataaagtatggtaattagttkkaaatgaaaagttacaaaytgatgagagctccatgaggattattgttgattaaaaaggaca

SEQ ID NO: 221 - DNA, 521 bp, Cannabis sativa
tgtcyaagaaaattggtttgcaaattcraactcgaagcggtggtcatgattmtgargrcatgtcmtacatatctcaagtccmatwtgttatagtagayttgagaaacatgcattcaatcaamatagrtgtycatasccaaayygcawgggttgaagcyrgagctacccttggagaagtttattattgggttaatgagaaaaatgrgartcttagtttggckgctgggtattgccctactgttwgcgcagstggacactttggtggrggaggmtatggaccattgatkmraarytatggcctygcrgctgataatatcrttgatgcacacttagtcaacrttsatgsaaaagtgctagatcgaaaatmtatgggrgaagatctcttttgggctwtacgtkgtggtggagsagaaagcttcrgaatcattgtagmatggaaaattagaytggttgctgtcccaamaaagtctactatgtttagtgttaaaaagatcatggagatacatgagcttgtcaagtka

SEQ ID NO: 222 - DNA, 891 bp, Cannabis sativa
atgggrsaagatctcttttgggctatacgtkgyggtggargagaaagcttcagaatcattgtagcayggaaaattagaytkgttgctrtcccamcaaagtctactrtgyttagtgttaaaaagatcaaggagatacatgagcttgtgaarttagttaacaagtggcaaaayatttcttacaagtatsacawagatttattactcatgactcacttyataacyarraayaytayagataatcaaggraagaataaracaayartacacrsttacttcyckttagttttccttkgtggagtsgatagtctagtcgamttratgaacaagagttttcctgagytkgkyattaaaraaayrgattgcaracaattgagctggattgatacyatcatcttctatagyggtgttgtaaattacgrcactgataattttaataassaaatttygcttgwtagatcagctgggcagaacggtgctttcaagattaagttagactaygttaagaaaccaattccagaatctgcrtttgtcaaaattttkgaaaaattatatgaagaagatraaggagttgggatgtatgcgttgtacmcttacggttgymtaatggatgagatttcagaatcagcaattccattccctcatcgagttrgaatcatgtatgaattatrgtacayatgtarctggragaagcamgaagataamgaaaagyatctaaactggattmsaaatrttkataacttcatgactccttatgtgtcccaaartccaagattgrcatatctcaattatagacaycttgatataggaataaatgatcccaagagtcaaaataattacacasaagcatgtatttkgggtgagaaatctttggtaaawattttgacaggctag

SEQ ID NO: 223 - DNA, 1320 bp, Cannabis sativa
attcgaactcgaagcggtggtcatgatgctgagggcatgtcctacatatctcaagtcccatttgttatagtaracttgagaaacatgcattcggtcaaaatagatgttcatagcsaaactgcatgggttgaagcmggagctacccttggagaagtttattattggatcaatgagaacaatgagaatcttagttttcytgctgggtactgccctactgttggcgcgggtggacactttagtggaggaggctatgragcattgatgcgaaattatggcctcgcggctrawaayatcattgatgcgcacttagtcaatgttgatggaaaagttttagatcgaaaatccatgggggaagatttgttttgggctatacgtggtggtggaggagaaaactttggaatcattgtagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtstagtcgacttgatgaacaagagctttcctgaattgggtattaaaaaaatagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaattttgagagacctaataattacacacaagcacgtatttggggtgaaaagtattttggtaaaaattttaataggttagtaaaagtaaaaaccaaggttgatcccgataatttctttagaaacgaacaaagcatcccacctcttcccctgcgtcatcattaa

SEQ ID NO: 224 - DNA, 1349 bp, Cannabis sativa
atgtctatcttaaattcgaccgtacaaaatcttagatttacctctgacacaaccccaaaaccacttgttatcaccactcctttaaatgtctcccatatccaaggcactattctatgttccaagaaagttgkcttgcagattyraactcgaagcggtggtcatgatgctgagggcatgtcctacatatctcaagtcccatttgttatagtagacttgagaaacatgcattyggtcaaaatagatgttcatagccaaactgcatgggttgaatccggagctacccttggagaagtttatwattggatcaatgagaacaatgagaatcttagttttcctgctgggtactgccctactgttggcrcgggtggrcactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaaaagrttgcaaacagttgagctggattgatactatcatcttctacagtggtsttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgakctggaatcacgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagta

SEQ ID NO: 225 - DNA, 1284 bp, Cannabis sativa; identical to SEQ ID NO: 190

SEQ ID NO: 226 - DNA, 1283 bp, Cannabis sativa
atgtcctacatatctgaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctacgtcatcatta

SEQ ID NO: 227 - DNA, 595 bp, Cannabis sativa
tttacctytgacacaaccccaaaaccacttgttatcatcrctcctttaaatgtctcccatatccaaggcactattctatgctccaagaaagttggcttgcagattcgagctcgaagcagtggtcatgatgctkagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgtcctactgttggygcgggtggacactttagtggaggaggctatggagcattgatgcgaaattatggcctcgcggctgataatatcattgatgcgcacttagtcaatgttgatggaaaagttttagatcgaaartccatggrggaagatttgttttgggctatacgtggtggtggagsagaaaactctggaatcattgcagcgtggaaaattagacttgttgctgtcccatcaatgtctactatattcagtgttaaaaagaacatggagatacatgagcttgtca

SEQ ID NO: 228 - DNA, 1458 bp, Cannabis sativa
atgtctatcytraattcgaccatacaaaatcttagatttacctytracacaaccccaaaaccacttgttatcatcactcctttaaatgtctcycatatccaaggcactattcwatgctccaagaaakttggcttgyagattcgaactcgaagcggtggtcatgatgctgagggcatgtcctacatatctcaagtcccrtttgttatagtaracttgagaaacatgcattcggtcaaaatagatgttcatagccaaamtgcatgggttgaagccggagctacccttggagaagtttattattggatcaatgagaayaatgagaatcttagttttcctgctgggtactgccctactgttggcgcwkgtggwcactttagtggaggaggctatggagcattratrcgaaattatggcctcgcrgctrataatatcattgatgcacacwtagtcaatgttgatggaaaagttytagatcgaaaatccatgggggaagatytrttttgggctatacgtggtgrtggaggwgaaaactttggaatcattgcagcgtggaaaattagactkgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattraaaaaagagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttytttagaaacgaacaaagcatyccacctcttcccctgcgtcatcattaa

SEQ ID NO: 229 - DNA, 450 bp, Cannabis sativa
atggtcacaattttggaaaaattatatgaagaagatgtaggagttgggatgtttgtgttttacccttatggtggwataatggatgagatttyagaatcagcaattccattccctcatcgagctggaatcatgtatgaaatttggtacatagcttcatgggagaagcaagaagataatgaaaagcatataaactggattcrgaatgtttacartttcacgactccttatgtgtcycaaaatccaagaatggcgtatctyaattatagggaccttgatttaggaaaaactaatttcgagagtcctaataattacacacaagcacgtatttggggtgaaaagtattttggtaaaaawtttaataggttagtaaaagtaaaaaccaaggttgatcccgrtaatttctttagaaacgaacaaagcatcccacctcttcccctacrtcatcattaa

SEQ ID NO: 230 - DNA, 1457 bp, Cannabis sativa
atgtctmtcctgaattcgacaatacaaaatcttagattcatctctgatacaaccccaaaaccactcgttattgtcactccttcaaataactcccatatccaagcaactattttatgctctaagaaagttggcttgcagwttcgaactcgaagcggtggccatgatgctgagggtwtgtcctacaywtctcaastcccattygttrtagtagacttgagraacatrcattygrtsaaaatagwtrttcrtagccaaaytgcrtgggttgaagcyggagctacccttggagaagtttaytwyyrgatyaatgagaagaatgagaatcttarttttcctggtgggtwttgccctactgttggcgymggtggacactttagtggaggaggctatrgakcattratrcgaaattatggcctygcrgctrataatatyattgatgcacacwtagtcaatgttgatggaaarkttctagatcgaaaatccrtgrgrgaagatmtrttttgggctatacgtggtggtggaggwgaaaactttggaatmattrcasyrtggaaaatyarmctggttsmtgtcmyrtcamrgkctactatattcagtgttraaargaayatggagatacatgggcttgtcaagttatttrayaaatggcaaaatattgcttacaagtatgacaaagatttaktactcatgactcacttcataacmmrraataytayagataatcawggraagaataaracwacagtacaygrttacttcycwtsmattttycatggyggagyrgatagtytrgtyracttgatraacaagagctttcstgagttgggtattaaaaaaactgattgcaaagaattkagctgrattgatacmaccatcttctacagtggwrttgtaaattwtaacactrctaattttmaaaaggaaattttgcttgatmgatcagctgggmmgaarryrrctttctcarttaagttagactaygttaagmamccaattccrraaayygcaatkgtcaaaattttggaraaattrtatgaagaagatgtagragytggrrtgtatstrttgwwcccktrcggyggtayaatggakragatytcagaatcarcaattccwttccstcatcgagctggaatmatgtaygwastttgrtacrcwgctwcctgggagaagcaagaagataatgaaaagcatataaactgggttcgaagtgtwtataatttyaygackccttatgtgtcccaaaatccaagawtggcgtatctcaattatagggaccttgatttaggaaaaactaatcmyrsgagtcctaataattacacacaagcaygtatttggggtgaaaagtattttrgtaaaaattttaacaggttwgttaargtgaaaacyaargttgatcccaataatttttttagaaacgaacaaagyatcmcamctcttccacygcrtcrtcmtta

SEQ ID NO: 231 - DNA, 1457 bp, Cannabis sativa
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaattcatggkagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcatta

SEQ ID NO: 232 - DNA, 1458 bp, Cannabis sativa; identical to SEQ ID NO: 197

SEQ ID NO: 233 - DNA, 980 bp, Cannabis sativa
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatccatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagatta

(19) United States
(12) Patent Application Publication
(10) Pub. No.: US 2023/0125534 A1
(43) Pub. Date: Apr. 27, 2023

(54) A Method to Produce Targeted Gene Editing Constructs

(71) Applicant: Agriculture Victoria Services Pty Ltd, Bundoora (AU)

(72) Inventors: Ehab Ibrahim Ahmed Mohamaden, Williamstown (AU); Lennon James Matchett-Oates, Preston (AU); Shivraj Kaur Braich, Glenroy (AU); Noel Cogan, Macleod (AU); German Carlos Spangenberg, Bundoora (AU)

(21) Appl. No.: 17/767,300

(22) PCT Filed: Oct. 14, 2020
(86) PCT No.: PCT/AU2020/051102

§ 371 Date: Apr. 7, 2022

(30) Foreign Application Priority Data
Nov. 4, 2019 (AU) 2019904146

(51) Int. Cl. C12N 15/82; C12N 9/22

(52) U.S. Cl. CPC C12N 15/8243; C12N 15/8216; C12N 9/22

(57) ABSTRACT

The present disclosure relates generally to targeted gene editing constructs, including methods of designing a DNA-recognition moiety for modulation of gene expression in plants, DNA-recognition moieties, gene editing constructs, methods for the modulation of gene expression in plants using gene editing constructs, and plants or regenerable plant cells produced therefrom.

### FIELD

The present disclosure relates generally to targeted gene editing constructs, including methods of designing a DNA-recognition moiety for modulation of gene expression in plants, DNA-recognition moieties, gene editing constructs, methods for the modulation of gene expression in plants using gene editing constructs, and plants or regenerable plant cells produced therefrom.

### RELATED APPLICATIONS

This application claims priority from Australian Provisional Patent Application No. 2019904146 filed on 4 Nov. 2019, the entire content of which is hereby incorporated by reference.

### SEQUENCE LISTING

This application contains a Sequence Listing, which has been submitted electronically and is hereby incorporated by reference in its entirety. Nucleotide bases are defined in accordance with the International Union of Pure and Applied Chemistry (IUPAC) nucleic acid notation, which is consistent with the World Intellectual Property Organization (WIPO) Handbook on Industrial Property Information and Documentation, Standard ST.25.

### BACKGROUND

Cannabis sativa is an herbaceous flowering plant of the Cannabis genus (Rosale) that has been used for its fibre and medicinal properties for thousands of years. The medicinal qualities of cannabis have been recognised since at least 2800 BC, with use of cannabis featuring in ancient Chinese and Indian medical texts. Although use of cannabis for medicinal purposes has been known for centuries, research into the pharmacological properties of the plant has been limited due to its illegal status in most jurisdictions.

The chemistry of cannabis is varied. It is estimated that cannabis plants produce more than 400 different molecules, including phytocannabinoids, terpenes and phenolics. Cannabinoids, such as Δ-9-tetrahydrocannabinol (THC) and cannabidiol (CBD) are the most well-known and researched cannabinoids. CBD and THC are naturally present in their acidic forms, Δ-9-tetrahydrocannabinolic acid (THCA) and cannabidiolic acid (CBDA) in planta, which are alternative products of a shared precursor, cannabigerolic acid (CBGA). Since different cannabinoids are likely to have different therapeutic potential, it is important to be able to identify and extract different cannabinoids that are suitable for medicinal use.

Despite advances in plant breeding technologies and the increasing commercial importance of cannabis plant varieties, there remains a need for improved methods of producing cannabis plants with one or more desirable phenotypic and/or chemotypic traits, including for large-scale production and breeding programs.

### SUMMARY

In an aspect disclosed herein, there is provided a method of producing a nucleic acid sequence encoding a DNA-recognition moiety for a targeted gene editing construct, the method comprising:
a. providing a nucleic acid sequence of a genome from a plant of a reference species;
b. providing a corresponding nucleic acid sequence of a genome from one or more additional plants of the reference species;
c. generating a consensus sequence of the nucleic acid sequences of (a) and (b);
d. identifying regions of genetic variation within the consensus sequence of (c); and
e. producing a nucleic acid sequence encoding a DNA-recognition moiety that is complementary to a target DNA sequence within the consensus sequence of (c), wherein the DNA-recognition moiety is not complementary to a region of genetic variation identified in (d).

In another aspect disclosed herein, there is provided a nucleic acid sequence encoding a DNA-recognition moiety produced by the methods disclosed herein.

In another aspect disclosed herein, there is provided a gene editing construct comprising the nucleic acid sequence encoding the DNA-recognition moiety disclosed herein.

In another aspect disclosed herein, there is provided a method of modulating gene expression in a plant cell, the method comprising:
a. providing a plant cell;
b. transfecting the plant cell with the gene editing construct disclosed herein;
c. culturing the transfected plant cell of (b) for a time and under conditions suitable to drive the functional expression of the gene editing construct in the plant cell.

In another aspect disclosed herein, there is provided a transformed plant cell comprising the gene editing construct disclosed herein.

In another aspect disclosed herein, there is provided a method for producing a regenerable plant cell with modified gene expression, the method comprising:
a. providing germinated plant tissue comprising regenerable cells;
b. transforming the regenerable cells with a gene editing construct disclosed herein;
c. culturing the transformed regenerable cells of (b) for a time and under conditions suitable to drive the functional expression of the gene editing construct in the regenerable cells;
d. culturing the transformed regenerable cells of (c) for a time and under conditions suitable for callus formation to occur; and
e. culturing the callus formed in (d) for a time and under conditions suitable to produce a rooted plantlet, wherein the rooted plantlet is capable of growing into a plant with modified gene expression.

In another aspect disclosed herein, there is provided a plant comprising the transformed plant cell described herein.

In another aspect disclosed herein, there is provided a regenerable plant cell produced according to the methods disclosed herein.

### BRIEF DESCRIPTION OF THE FIGURES

FIG. 1 is a schematic representation of the design of single-guide (sgRNA).

FIG. 2 is a photographic representation of the surface sterilisation of Cannabis apical buds.

FIG. 3 is a photographic representation of the removal of sterilisation agent from Cannabis apical buds.

FIG. 4 is a photographic representation of sterilised Cannabis apical buds in regeneration medium.

FIG. 5 is a photographic representation of sterilised Cannabis apical buds displaying various tags of regrowth in regeneration medium.

FIG. 6 is a photographic representation of regenerated Cannabis plant removed from regeneration medium and ready to transfer to solid plant growth medium or to transfer to fresh tissue culture medium.

FIG. 7 is a photographic representation of the source of protoplasts, from leaf mesophylls of healthy, rooted plants from in vitro culture.

FIG. 8 is a photographic representation of Cannabis leaf material before (left panel), during (middle panel) and after (right panel) treatment with a solution of 2% Cellulase+0.5% Macerozyme R-10+0.2% Pectolase.

FIG. 9 is a photographic representation of mechanically filtrated, digested leaf protoplasts and associated successive filtration steps to remove remaining plant debris.

FIG. 10 is a photographic representation of purified Cannabis leaf mesophyll protoplasts.

FIG. 11 is a photographic representation of purified, isolated Cannabis protoplasts under microscopic magnification showing cell size and intactness and lack of debris and contaminating cellular waste.

FIG. 12 is a photographic representation of Cannabis mesophyll protoplasts transiently expressing GFP (upper panel) and Ds-RED (bottom panel) under fluorescent microscopy.

FIG. 13 is a graphical representation and report from the FACS analysis of protoplasts transfected with Ds-RED reporter gene.

FIG. 14 is a photographic representation of Cannabis seeds at surface sterilisation stage with the active agent removed with subsequent washes.

FIG. 15 is a photographic representation of Cannabis seeds at initial germination stage and then after 3 days imbibing with sterile water to initiate germination.

FIG. 16 is a photographic representation of Cannabis seeds at initial germination stage, showing radicle emergence.

FIG. 17 is a photographic representation of embryogenic cotyledons and initial callus induced from undifferentiated cotyledons.

FIG. 18 is a photographic representation of transformed embryogenic cotyledon of Cannabis inoculated with Agrobacterium strain EHA105 containing a Ti plasmid with Ds-RED as a reporter gene construct driven by the 35S promoter.

FIG. 19 is a photographic representation of embryogenic callus.

FIG. 20 is a photographic representation of regenerating callus displaying shoot formation.

FIG. 21 is a photographic representation of a regenerating plantlet derived from regenerating callus.

FIG. 22 is a photographic representation of mature plant derived from tissue culture containing edited genome.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “an endonuclease” includes a single endonuclease, as well as two or more endonuclease, and so forth.

As used herein, “and/or” refers to and encompasses any and all possible combinations of one or more of the associated listed items, as well as the lack of combinations when interpreted in the alternative (or).

The present disclosure is predicated, at least in part, on the unexpected finding that multiple reference genomes may be used to construct a pan-genome useful in the production of high-efficiency gene editing constructs, while minimising the possibility of off-target effects. Such gene editing constructs may be used in advantageous plant production methods, including the screening of gene editing constructs for the efficient and effective modulation of plant gene expression in transient in vitro plant cell models, and the stable transformation of plant cells capable of regenerating a whole plant with modified gene expression as a result of the expression of the gene editing constructs.

Accordingly, in an aspect disclosed herein, there is provided a method of producing a nucleic acid sequence encoding a DNA-recognition moiety for a targeted gene editing construct, the method comprising:
a. providing a nucleic acid sequence of a genome from a plant of a reference species;
b. providing a corresponding nucleic acid sequence of a genome from one or more additional plants of the reference species;
c. generating a consensus sequence of the nucleic acid sequences of (a) and (b);
d. identifying regions of genetic variation within the consensus sequence of (c); and
e. producing a nucleic acid sequence encoding a DNA-recognition moiety that is complementary to a target DNA sequence within the consensus sequence of (c), wherein the DNA-recognition moiety is not complementary to a region of genetic variation identified in (d).

The term “DNA-recognition moiety” as used herein refers to a molecule that is capable of hybridising to a target DNA sequence, or its complement, for use in gene editing.

Preferred DNA-recognition moieties will hybridise under stringent conditions to a target DNA sequence, or its complement. The term “hybridise under stringent conditions”, and grammatical equivalents thereof, refers to the ability of a nucleic acid molecule to hybridise to a target nucleic acid molecule under defined conditions of temperature and salt concentration. With respect to nucleic acid molecules greater than about 100 bases in length, typical stringent hybridisation conditions are no more than 25° C. to 30° C. (for example, 10° C.) below the melting temperature (Tm) of the native duplex (see generally, Sambrook et al., supra). Tm for nucleic acid molecules greater than about 100 bases can be calculated by the formula Tm=81.5+0.41% (G+C-log (Nat)). With respect to nucleic acid molecules having a length less than 100 bases, exemplary stringent hybridisation conditions are 5° C. to 10° C. below Tm.

Persons skilled in the art would understand that the DNA-recognition moiety may be DNA, RNA or a polypeptide.

Illustrative examples of suitable DNA molecules include antisense, as well as sense (e.g., coding and/or regulatory) DNA molecules. Antisense DNA molecules include short oligonucleotides. Other examples of inhibitory DNA molecules include those encoding interfering RNAs, such as shRNA and siRNA. Yet another illustrative example of an inhibitor of gene expression is catalytic DNA, also referred to as DNAzymes.

Illustrative examples of suitable RNA molecules include siRNA, dsRNA, stRNA, shRNA and miRNA (e.g. short temporal RNAs and small modulatory RNAs), ribozymes, and guide (i.e., gRNA or single-guide RNA (sgRNA)) or clustered regularly interspaced short palindromic repeats (CRISPR) RNAs used in combination with the Cas or other endonucleases (van der Oost et al. 2014, Nature Reviews Microbiology,12(7):479-92).

In an embodiment, the DNA-recognition moiety is a CRISPR RNA. Suitable CRISPR RNA will be known to persons skilled in the art, illustrative examples of which include guide RNA (gRNA) and single-guide RNA (sgRNA).

In an embodiment, the DNA-recognition moiety is a polypeptide. Illustrative examples of a suitable polypeptide molecules are zinc finger nucleases or “ZFN”, and transcription activator-like (TAL) targeting domains, as described elsewhere herein.

The terms “guide RNA” or “gRNA” refer to a RNA sequence that is complementary to a target DNA and directs a CRISPR endonuclease to the target DNA. gRNA comprises crispr RNA (crRNA) and a tracr RNA (tracrRNA). crRNA is a 17-20 nucleotide sequence that is complementary to the target DNA, while the tracrRNA provides a binding scaffold for the endonuclease. crRNA and tracrRNA exist in nature a two separate RNA molecules, which has been adapted for molecular biology techniques using, for example, 2-piece gRNAs such as CRISPR tracer RNAs (cr:tracrRNAs).

The terms “single-guide RNA” or “sgRNA” refers to a single RNA sequence that comprises the crRNA fused to the tracrRNA.

Accordingly, the skilled person would understand that the term “gRNA” describes all CRISPR guide formats, including two separate RNA molecules or a single RNA molecule. By contrast, the term “sgRNA” will be understood to refer to single RNA molecules combining the crRNA and tracrRNA elements into a single nucleotide sequence.

In a preferred embodiment, the DNA-recognition moiety is a single-guide RNA (sgRNA).

Methods to optimise the design and efficiency of sgRNAs will be known to persons skilled in the art, illustrative examples of which include the paired nicking strategy described by Cho et al. (2014, Genome Research, 24: 132-41) and Ran et al. (2013, Cell, 154: 1380-9), dimeric-Cas9 based systems as described by Wyvekens et al. (2015, Human Gene Therapy, 26: 425-31), truncation of the 3′ end of the sgRNA scaffold as described by Hsu et al. (2013, Nature Biotechnology, 31: 827-32), or addition of two guanine nucleotides to the 5′ end of the sgRNA as described by Cho et al. (2014, supra). The length of the sgRNA has also been demonstrated to result in different effects of CRISPR-mediated modification of gene expression (Zhang et al., 2016, Scientific Reports, 6: 28566).

In an embodiment, the sgRNA is complementary to a target DNA sequence of between 10 and 30 nucleotides in length.

In an embodiment, the sgRNA consists of a sequence provided in Table 5, or complementary sequences thereof.

In an aspect disclosed herein, there is provided a DNA-recognition moiety produced according to the methods disclosed herein.

The term “targeted gene editing construct” as used herein refers to a recombinant nucleic acid molecule formed in vitro by the manipulation of nucleic acid into a form not normally found in nature.

In an embodiment, the targeted gene editing construct is an expression vector.

The term “vector” as used herein refers to a nucleic acid molecule, preferably a DNA molecule derived from a plasmid or plant virus, into which a nucleic acid sequence may be inserted. The vector may also include a selection marker such as an antibiotic resistance gene that can be used for selection of suitable bacterial or plant transformants, or sequences that enhance transformation of prokaryotic or eukaryotic (especially cannabis) cells such as T-DNA or P-DNA sequences. Examples of such resistance genes and sequences are well known to those of skill in the art.

In an embodiment, the targeting gene editing construct is a plasmid. In another embodiment, the plasmid is a Ti plasmid.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The term “endogenous” refers to a gene or nucleic acid sequence or segment that is normally found in a host organism.

The terms “expressible,” “expressed,” and variations thereof refer to the ability of a cell to transcribe a nucleotide sequence to RNA and optionally translate the mRNA to synthesise a peptide or polypeptide that provides a biological or biochemical function.

As used herein, the term “gene” includes a nucleic acid molecule capable of being used to produce mRNA optionally with the addition of elements to assist in this process. Genes may or may not be capable of being used to produce a functional protein. Genes can include both coding and non-coding regions (e.g., introns, regulatory elements, promoters, enhancers, termination sequences and 5′ and 3′ untranslated regions).

The terms “heterologous nucleic acid sequence,” “heterologous nucleotide sequence,” “heterologous polynucleotide,” “foreign polynucleotide,” “exogenous polynucleotide” and the like are used interchangeably to refer to any nucleic acid (e.g., a nucleotide sequence encoding at least one targeting RNA), which is introduced into the genome of an organism by experimental manipulations.

The terms “heterologous polypeptide,” “foreign polypeptide” and “exogenous polypeptide” are used interchangeably to refer to any peptide or polypeptide, which is encoded by a “heterologous nucleic acid sequence,” “heterologous nucleotide sequence,” “heterologous polynucleotide,” “foreign polynucleotide” and “exogenous polynucleotide,” as defined above.

The term “operably connected” or “operably linked” as used herein refers to a juxtaposition wherein the components so described are in a relationship permitting them to function in their intended manner. For example, a regulatory element or regulatory sequence “operably linked” to a coding sequence refers to positioning and/or orientation of the regulatory sequence relative to the coding sequence to permit expression of the coding sequence under conditions compatible with the regulatory sequence.

By “regulatory element” or “regulatory sequence” it is meant a nucleic acid sequence (e.g., DNA) necessary for expression of an operably linked coding sequence in a particular host cell. The regulatory sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

In an embodiment, the regulatory element is a promoter. In another embodiment, the promoter is a 35S promoter.

The terms “polynucleotide,” “polynucleotide sequence,” “nucleotide sequence,” “nucleic acid” or “nucleic acid sequence” as used herein designate mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of RNA or DNA.

“Polypeptide,” “peptide,” “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers.

As used herein the term “recombinant” as applied to “nucleic acid molecules,” “polynucleotides” and the like is understood to mean artificial nucleic acid structures (i.e., non-replicating cDNA or RNA; or replicons, self-replicating cDNA or RNA) which can be transcribed and/or translated in host cells or cell-free systems described herein. Recombinant nucleic acid molecules or polynucleotides may be inserted into a vector. Non-viral vectors such as plasmid expression vectors or viral vectors may be used. The kind of vectors and the technique of insertion of the nucleic acid construct would be known to persons skilled in the art. A nucleic acid molecule or polynucleotide according to this disclosure does not occur in nature in the arrangement described by the present invention. In other words, a heterologous nucleotide sequence is not naturally combined with elements of a parent virus genome (e.g., promoter, ORF, polyadenylation signal, DNA-recognition moiety, endonuclease).

In an embodiment, the targeting gene editing construct further comprises a nucleic acid encoding an endonuclease.

Suitable endonucleases will be known to persons skilled in the art, illustrative examples of which include an RNA-guided DNA endonuclease, zinc finger nuclease (ZFN), transcription activator-like effector nucleases (TALEN), CRISPR-associated (Cas) nucleases.

In an embodiment, the nuclease is selected from the group consisting of an RNA-guided DNA endonuclease, ZFN, and a TALEN.

“Transcription activator-like effector nucleases” or “TALEN” are restriction enzymes that can be engineered to cut specific sequences of DNA. They are made by fusing a TAL effector DNA-binding domain to a DNA cleavage domain (a nuclease that cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. The mechanism of TALEN-mediated cleavage of target DNA sequences would be known to persons skilled in the art and has been described, for example by Boch (2011, Nature Biotechnology, 29: 135-136), Juong et al. (2013, Nature Reviews Molecular Cell Biology, 14: 49-55) and Sune et al. (2013, Biotechnology and Bioengineering, 110: 1811-1821).

“Zinc finger nucleases” or “ZFN” are proteins comprising nucleic acid binding domains that are stabilised by zinc. The individual DNA binding domains are typically referred to as “fingers”, such that a ZFN has at least one finger, preferably two fingers, preferably three fingers, preferably four fingers, preferably five fingers, or more preferably six fingers. Each finger binds from two to four base pairs of a target DNA sequence, and typically comprises an about 30 amino acid zinc-chelating, DNA binding region. ZFN facilitate site-specific cleavage within a target DNA sequence, allowing endogenous or other end-joining repair mechanisms to introduce insertions or deletions to repair the gap. The mechanism of ZFN-mediated cleavage of target DNA sequences would be known to persons skilled in the art and has been described, for example, by Liu et al. (2010, Biotechnology and Bioengineering, 106: 97-105).

In an embodiment, the RNA-guided DNA endonuclease is a CRISPR-associated (Cas) endonuclease.

The CRISPR-Cas system evolved in bacteria and archaea as an adaptive immune system to defend against viral attack. Upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence.

The mechanisms of CRISPR-mediated gene editing would be known to persons skilled in the art and have been described, for example, by Doudna et al., (2014, Methods in Enzymology, 546) and Belhaj et al., (2013, Plant Methods, 9:39) and in WO 2013/188638 and WO 2014/093622.

Suitable Cas endonucleases will be known to persons skilled in the art, illustrative examples of which include Cas9, Cas12a (also referred to as Cpf1), Cas12b (also referred to as C2c1), Cas13a (also referred to as C2c2), Cas13b, CasX, Cas3 and Cas10. The term “Cas endonucleases” as used herein also contemplates the use of natural and engineered Cas endonucleases, described, for example, by Wu et al. (2018, Nature Chemical Biology, 14: 642-651).

In a preferred embodiment, the Cas endonuclease is Cas9.

In an aspect, the present disclosure provides a gene editing construct comprising a nucleic acid sequence encoding the DNA-recognition moiety disclosed herein.

In an embodiment, the gene editing construct further comprises a nucleic acid encoding an endonuclease.

The term “genome” as used herein refers to the total inherited genetic complement of the cell, plant or plant part, and includes chromosomal DNA, plastid DNA, mitochondrial DNA and extrachromosomal DNA molecules.

In an embodiment, the genome is a de novo assembled genome sequence. In another embodiment, the genome is a published assembled genome sequence.

A skilled person would understand that genomes for use in accordance with the methods disclosed herein may be derived from both male and female plants of a reference species.

The terms “consensus sequence” or “canonical sequence” may be used interchangeably herein to refer to a nucleic acid sequence that represents the most frequent residues of a nucleic acid sequence found at each position in a sequence alignment. Accordingly, the skilled person would understand that the consensus sequence described herein represents the result of the comparison between the nucleic acid sequence of a genome from a plant of a reference species, with the corresponding nucleic acid sequence of a genome from one or more additional plants of the reference species. Methods for comparison of nucleic acid sequences would be known to persons skilled in the art, illustrative examples of which include multiple sequence alignment.

Multiple sequence alignment may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as described by, for example, Altschul et al. (1997, Nucleic Acids Research, 25:3389). A detailed discussion of sequence analysis can be found in Unit 19.3 of Ausubel et al., supra.

In an embodiment, sequences of similar length with an alignment similarity of between 80 and 100% are incorporated into the consensus sequence. The term “between 80 and 100%” as used herein means preferably about 80%, preferably about 81%, preferably about 82%, preferably about 83%, preferably about 84%, preferably about 85%, preferably about 86%, preferably about 87%, preferably about 88%, preferably about 89%, preferably about 90%, preferably about 91%, preferably about 92%, preferably about 93%, preferably about 94%, preferably about 95%, preferably about 96%, preferably about 97%, preferably about 98%, preferably about 99%, or more preferably about 100% alignment similarity.

The term “pan-genome” as used herein refers to the entire gene set of all strains in a species. It includes genes present in all strains (i.e., the core genome) and genes present only in some strains of a species (i.e., variable or accessory genome). The core genome represents the genes present in all strains of a species. It typically includes housekeeping genes for cell envelope or regulatory functions. The variable or accessory genome refers to genes not present in all strains or species. These include genes present in two or more strains or even genes unique to a single strain only, for example, genes for a strain specific adaptation, such as increased expression of a particular cannabinoid (e.g., THC and/or CBD).

In an embodiment, the consensus sequence is a Cannabis sativa pan-genome.

The term “genomic variation” as used herein refers to differences in the genomes of a plant from a reference species, as compared to the genomes from one or more additional plants of the reference species.

In an embodiment, the genome variation is selected from the group consisting of a single nucleotide polymorphism (SNP) location, SNP frequency, copy number variation (CNV) and presence absence variations (PAV).

In an embodiment, the genomic variation is a genomic variation shown in any one of the sequences selected from the group consisting of SEQ ID NO: 199-233.

The term “polymorphism” refers to any change in the nucleotide sequence of the gene including such silent nucleotide substitutions.

A “single nucleotide polymorphism” or “SNP” is a substitutional variant that occurs are a specific position in the genome. Substitutional nucleotide variants are those in which at least one nucleotide in the sequence has been removed and a different nucleotide inserted in its place. In some embodiments, the number of nucleotides affected by substitutions in a mutant gene relative to the wild-type gene is a maximum of ten nucleotides, more preferably a maximum of 9, 8, 7, 6, 5, 4, 3, or 2, or most preferably only one nucleotide. Substitutions may be “silent” in that the nucleotide substitution does not change the amino acid defined by the codon. Alternatively, the nucleotide substitution(s) may change the encoded amino acid sequence and thereby alter the activity of the encoded enzyme, particularly if conserved amino acids are substituted for another amino acid which is quite different i.e., a non-conservative substitution.

The term “copy number variation” or “CNV” is a duplication or deletion event that affects a number of base pairs. These structural variants result in a change in the number of copies of a particular gene between one reference genome and the next.

An allele is a variant of a gene at a single genetic locus. Each chromosome of a pair of chromosomes has one copy (i.e., one allele) of each gene. If both alleles of a gene are the same, the organism is homozygous with respect to that allele or gene. If the two alleles are different, the organism is heterozygous with respect to that gene. The two alleles of a gene in the plant may have the same mutation as each other, so are said to be homozygous for that mutation, or the two alleles may comprise different mutations to each other and are said to be heterozygous for those mutations.

### Cannabis

Cannabis is an erect annual herb with a dioecious breeding system, although monoecious plants exist. Wild and cultivated forms of Cannabis are morphologically variable, which has resulted in difficulty defining the taxonomic organisation of the genus.

In an embodiment, the reference species is of the genus Cannabis. Plants of the genus Cannabis will be known to persons skilled in the art, illustrative examples of which include Cannabis sativa, Cannabis indica and Cannabis ruderalis.

In an embodiment, the reference species is Cannabis sativa, also referred to as C. sativa.

In an embodiment, the reference species is Cannabis sativa, and wherein the genome of the reference species comprises one or more nucleic acid sequences selected from the group consisting of SEQ ID NOs: 164-198.

The terms “plant”, “cultivar”, “variety”, “strain” or “race” are used interchangeably herein to refer to a plant or a group of similar plants according to their structural features and performance (i.e., morphological and physiological characteristics).

The published reference genome for C. sativa is the assembled draft genome and transcriptome of “Purple Kush” or “PK” (van Bakal et al., 2011, Genome Biology, 12(10): R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

### Cannabinoids

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated forms thereof.

TABLE 1
Cannabinoids and their properties.

| | | Chemical |
|---|---|---|
| | | properties/ |
| | | [M + H]⁺ ESI |
| Name | Structure | MS |
| Δ9- tetrahydrocannabinol (THC) | 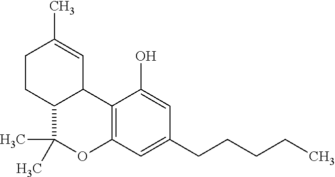 | Psychoactive, decarboxylation product of THCA m/z 315.2319 |
| Δ9- tetrahydrocannabinolic acid (THCA) | 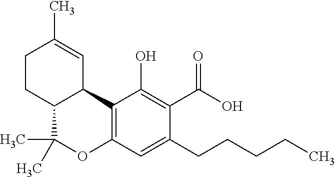 [SMILES: CCCCCc1cc2c(c(O)c1C(=O)O)[C@@H]1C=C(C)CC[C@H]1C(C)(C)O2] | m/z 359.2217 |
| cannabidiol (CBD) | 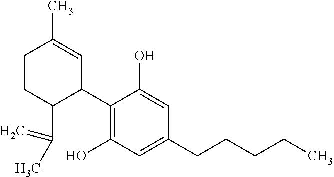 [SMILES: C=C(C)C1CCC(C)=CC1c1c(O)cc(CCCCC)cc1O] | decarboxylation product of CBDA m/z 315.2319 |
| cannabidiolic acid (CBDA) | 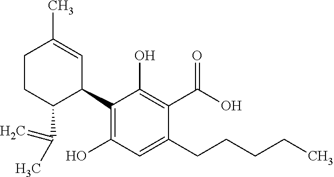 [SMILES: C=C(C)[C@@H]1CCC(C)=C[C@H]1c1c(O)cc(CCCCC)c(C(=O)O)c1O] | m/z 359.2217 |
| cannabigerol (CBG) | 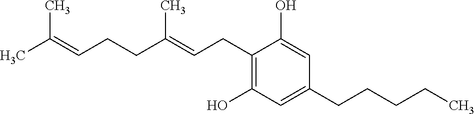 [SMILES: CCCCCc1cc(O)c(C/C=C(\C)CCC=C(C)C)c(O)c1] | Non- intoxicating, decarboxylation product of CBGA m/z 317.2475 |
| cannabigerolic acid (CBGA) | 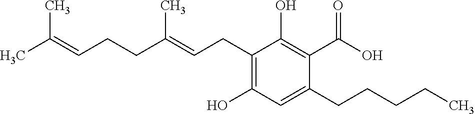 [SMILES: CCCCCc1cc(O)c(C/C=C(\C)CCC=C(C)C)c(O)c1C(=O)O] | m/z 361.2373 |
| cannabichromene (CBC) | 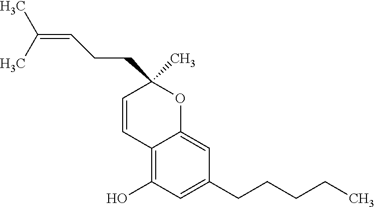 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 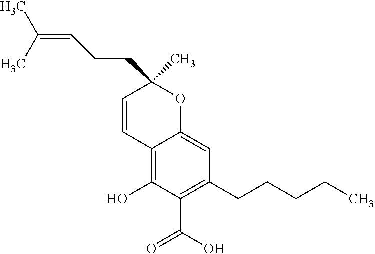 [SMILES: CCCCCc1cc2c(c(O)c1C(=O)O)C=C[C@@](C)(CCC=C(C)C)O2] | m/z 359.2217 |
| cannabicyclol (CBL) | 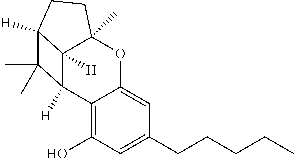 | Non- psychoactive, 16 isomers known. Derived from non- enzymatic conversion of CBC m/z 315.2319 |
| cannabinol (CBN) | 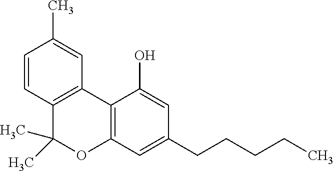 [SMILES: CCCCCc1cc(O)c2c(c1)OC(C)(C)c1ccc(C)cc1-2] | Likely degradation product of THC m/z 311.2006 |
| cannabinolic acid (CBNA) | 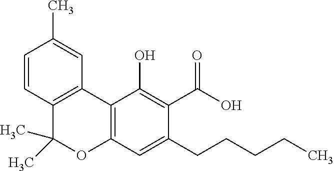 [SMILES: CCCCCc1cc2c(c(O)c1C(=O)O)-c1cc(C)ccc1C(C)(C)O2] | m/z 355.1904 |
| tetrahydrocannabivarin (THCV) | 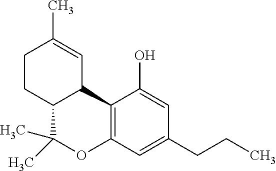 | decarboxylation product of THCVA m/z 287.2006 |
| tetrahydrocannabivarinic acid (THCVA) | 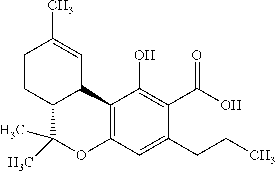 | m/z 331.1904 |
| cannabidivarin (CBDV) | 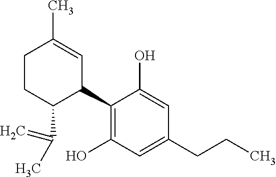 | m/z 287.2006 |
| cannabidivarinic acid (CBDVA) | 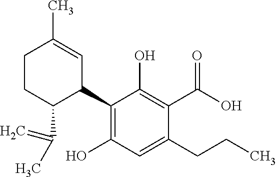 | m/z 331.1904 |
| Δ8-tetrahydrocannabinol (d8-THC) | 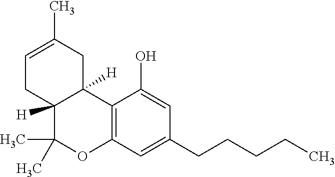 [SMILES: CCCCCc1cc(O)c2c(c1)OC(C)(C)[C@@H]1CC=C(C)C[C@@H]21] | m/z 315.2319 |

Cannabinoid biosynthesis in plants typically involves the production of fatty acid and isoprenoid precursors via the hexonate, methylerythritol 4-phosphate (MEP) and gernyl diphosphate (GPP) pathways, as described by, for example, Marks et al. (2009, Journal of Experimental Botany, 60: 3715).

The hexonate pathway involves desaturase, lipoxygenase (LOX), hydroperioxide lyase (HPL) and an acyl-activating enzyme (AEE) step that produces hexanoyl-CoA. Hexanoyl-CoA produced via the hexonate pathway acts as the substrate for polyketide synthase enzyme (OLS) that yields olivetolic acid.

The MEP pathway results in the synthesis of a prenyl side-chain, which is utilised as the substrate for GPP synthesis (Phillips et al., 2008, Trends in Plant Science, 13(12): 619-23). GPP is added by an aromatic prenyltransferase (PT) that yields CBGA (WO 2011/017798). The final steps involve catalysis by the oxidocyclases THCAS and CBDAS resulting in the production of THCA and CBDA, respectively (van Bakel et al., supra).

Cannabinoids are synthesised in cannabis plants as carboxylic acids. While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of cannabinoids can be promoted, illustrative examples of which include air-drying, combustion, vaporisation, curing, heating and baking.

### Terpenes

The term “terpene” as used herein, refers to a class of organic hydrocarbon compounds, which are produced by a variety of plants. Cannabis plants produce and accumulate different terpenes, such as monoterpenes and sesquiterpenes, in the glandular trichomes of the female inflorescence. The term “terpene” includes “terpenoids” or “isoprenoids”, which are modified terpenes that contain additional functional groups.

Terpenes are responsible for much of the scent of cannabis flowers and contribute to the unique flavour qualities of cannabis products. Terpenes will be known to persons skilled in the art, illustrative examples of which are provided in Table 2.

TABLE 2
Terpenes and their properties

| | | Mass/Charge number |
|---|---|---|
| Name | Structure | (m/z)* |
| α-Phellandrene | 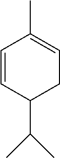 | m/z 93.0 |
| α-Pinene (+/−) | 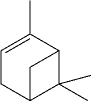 | m/z 93.0 |
| Camphene | 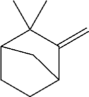 | m/z 93.0 |
| β-Pinene (+/−) | 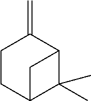 | m/z 93.0 |
| Myrcene | 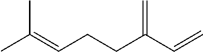 | m/z 93.0 |
| Limonene | 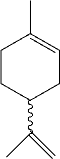 | m/z 68.1 |
| 3-Carene | 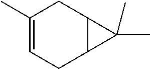 | |
| Eucalyptol | 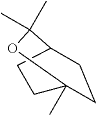 | m/z 81.0 |
| γ-Terpinene | 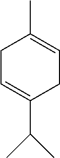 | m/z 93.1 |
| Linalool | 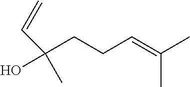 | m/z 93.0 |
| γ-Elemene | 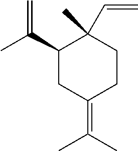 | m/z 121.0 |
| Humulene | 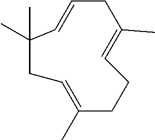 | m/z 93.0 |
| Nerolidol | 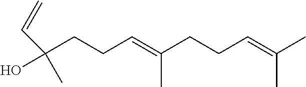 [SMILES: C=CC(C)(O)CC/C=C(\C)CCC=C(C)C] | m/z 222.4 |
| Guaia-3,9-diene | 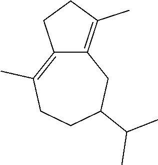 | m/z 161.1 |
| Caryophyllene | 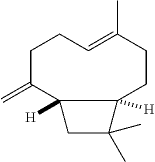 | m/z 69.2 |

*The molecular ion is not necessarily seen for all compounds

Terpene biosynthesis in plants typically involves two pathways to produce the general 5-carbon isoprenoid diphosphate precursors of all terpenes: the MEP pathway as described elsewhere herein, and the cytosolic mevalonate (MEV) pathway. These pathways control the different substrate pools available for terpene synthases (TPS).

### Cannabinoid Biosynthesis Genes

In an embodiment, the target DNA sequence comprises one or more cannabinoid biosynthesis genes.

Reference to “gene” includes DNA corresponding to the exons or the open reading frame of a gene. Reference herein to a “gene” is also taken to include a classical genomic gene consisting of transcriptional and/or translational regulatory sequences and/or a coding region and/or non-translated sequences (i.e., introns, 5′- and 3′—untranslated sequences), or mRNA or cDNA corresponding to the coding regions (i.e., exons) and 5′- and 3′—untranslated sequences of the gene.

The term “cannabinoid biosynthesis gene” as used herein refers to any gene encoding a protein involved in the biosynthesis of a cannabinoid.

In an embodiment, the cannabinoid biosynthesis gene is selected from the group consisting of DXS1, DXS2, DXR, MCT, CMK, MDS, HDS, HDR, IPP/IPI. GPP_LSU, GPP_SSU, FAD2#1, FAD2#2, FAD2#3, FAD2#4, LOX, HPL, AAE], OLS, OAC, OAC#2, GOT, CBCAS, CBCAS-like#a, CBCAS-like#b, CBCAS-like#c, CBCAS-like#d, CBCAS-like#e, CBCAS-like#f, CBCAS-like#g, CBCAS#a, CBCAS#b, and THCAS.

In an embodiment, the DNA-recognition moiety is complementary to a target sequence in at least one cannabinoid biosynthesis gene within the consensus sequence.

As described elsewhere herein, some aspects of terpene biosynthesis are also regulated by the MEP pathway, encoded by genes including DXS1, DXS2, MCT, CMK, HDS, HDR and GPPS. Accordingly, persons skilled in the art would understand that modulation of cannabinoid biosynthesis genes may also be useful in modulating the expression of some terpenes. Terpenes have been associated with therapeutic benefits independent from cannabinoids (Brahmkshatriya and Brahmkshatriya, 2013, in Ramawat and Merillon (eds), Natural Products, Springer, Berlin, Heidelberg). Therefore, modulation of terpene biosynthesis may also be advantageous for cannabis plant production.

Methods for Modulating Gene Expression

In an aspect disclosed herein, there is provided a method of modulating gene expression in a plant cell, the method comprising:
a. providing a plant cell;
b. transfecting the plant cell with the gene editing construct disclosed herein; and
c. culturing the transfected plant cell of (b) for a time and under conditions suitable to drive the function expression of the gene editing construct in the plant cell.

Modulation of gene expression by gene editing may be performed by introducing a targeted gene editing construct comprising a DNA-recognition moiety and an endonuclease that is capable of being functionally expressed in a cell to modifying gene expression. Accordingly, modulation of gene expression includes activating or inhibiting the expression of endogenous genes, inducing or enhancing the expression of endogenous genes and introducing and expressing one or more exogenous genes in a cell.

Modulation of gene expression in accordance with the methods disclosed herein may comprise the inhibition of gene expression or inducing or enhancing gene expression.

The term “inhibition of gene expression” and the like typically refer to a decrease in the level of mRNA in a plant cell as derived from a target DNA sequence (e.g., a cannabinoid biosynthesis gene). Such reduction may be the result of reduction of transcription, including by methylation of promoter regions via chromatin re-modelling, or post-transcriptional modification of the RNA molecules, including via RNA degradation, or both. Inhibition of gene expression should not necessarily be interpreted as an abolishing of the expression of the target nucleic acid or gene. In some embodiments, the introduction of a gene editing construct in a plant cell will decrease the level of mRNA by at least about 5%, preferably by at least about 10%, preferably by at least about 20%, preferably by at least about 30%, preferably by at least about 40%, preferably by at least about 50%, preferably by at least about 60%, preferably by at least about 70%, preferably by at least about 80%, preferably by at least about 90%, preferably by at least about 95%, preferably by at least about 99%, or preferably by about 100% of the mRNA level found in the plant cell in the absence of the gene editing construct.

Conversely, the term “inducing or enhancing gene expression” and the like refer to an increase in the level of mRNA in a plant cell for an endogenous (i.e., homologous or native) target gene (e.g., a cannabinoid biosynthesis gene). In some embodiments, the introduction of the gene editing construct in a cell will increase the level of endogenous mRNA by at least about 5%, preferably by at least about 10%, preferably by at least about 20%, preferably by at least about 30%, preferably by at least about 40%, preferably by at least about 50%, preferably by at least about 60%, preferably by at least about 70%, preferably by at least about 80%, preferably by at least about 90%, preferably by at least about 95%, preferably by at least about 99%, or preferably by about 100% of the mRNA level found in the cell in the absence of the gene editing construct.

Methods for the measurement of gene expression in plant cells would be known to persons skilled in the art, illustrative examples of which include RT-PCR, RNA-Seq, Northern blot analysis, and the like.

The modulation of gene expression in accordance with the methods disclosed herein may be stable, transient or conditional gene expression modulation.

In an embodiment, the gene editing construct transiently modulates the expression of one or more target genes in the plant cell.

In an embodiment, the gene editing construct stably modulates the expression of one or more target genes in the plant cell.

In an embodiment, the plant cell is a protoplast. In another embodiment, the protoplast is a mesophyll-derived protoplast.

In an aspect disclosed herein, there is provided a transformed plant cell comprising the gene editing construct as disclosed elsewhere herein.

### Methods for Screening Gene Editing Constructs

The inventors have surprisingly shown that in vitro propagated plant strains provide a source of mesophyll-derived protoplasts that are highly effective for the transient and rapid evaluation of gene editing constructs to identify effective gene editing constructs for use in the stable transduction of regenerable plant cells for the production of plants with modified gene expression.

Accordingly, in another aspect disclosed herein, there is provided a method for screening gene editing constructs in plant cells, comprising:
a. providing a plant cell;
b. transfecting the plant cell with a first gene editing construct comprising a nucleic acid encoding a DNA-recognition complementary to a target DNA sequence;
c. culturing the transfected plant cell for a time and under conditions suitable to drive the functional expression of the first gene editing construct in the plant cell;
d. determining the level of expression of the target DNA sequence in the transfected plant cell; and
e. repeating steps (a)-(d) with a second gene editing construct comprising a nucleic acid encoding a DNA-recognition complementary to the same target DNA sequence of (b);
f. comparing the level of expression of the target DNA sequence in plant cells transfected with the first gene editing construct and the second gene editing construct;
g. based on the comparison in step (f), identifying the most effective gene editing construct.

In an embodiment, the plant cell is a protoplast. In another embodiment, the protoplast is a mesophyll-derived protoplast.

Methods for producing a regenerable plant cell with modified gene expression

In an aspect disclosed herein, there is provided a method for producing a regenerable plant cell with modified gene expression, the method comprising:
a. providing germinated plant tissue comprising regenerable cells;
b. transforming the regenerable cells with a gene editing construct disclosed herein;
c. culturing the transformed regenerable cells of (b) for a time and under conditions suitable to drive the functional expression of the gene editing construct in the regenerable cells;
d. culturing the transformed regenerable cells of (c) for a time and under conditions suitable for callus formation to occur; and
e. culturing the callus formed in step (d) for a time and under conditions suitable to produce a rooted plantlet, wherein the rooted plantlet is capable of growing into a plant with modified gene expression.

A number of techniques are available for the introduction of nucleic acid molecules into regenerable cells derived from germinated plant tissue, well known to persons skilled in the art.

The term “transformation” as used herein means alteration of the genotype of a cell, for example, a bacterium or a plant, particularly a cannabis plant, by the introduction of a foreign or exogenous nucleic acid. By “transformant” is meant an organism so altered. Introduction of DNA into a plant by crossing parental plants or by mutagenesis per se is not included in transformation. The nucleic acid molecule may be replicated as an extrachromosomal element or is preferably stably integrated into the genome of the plant.

The most commonly used methods to produce fertile, transgenic plants comprise two steps: the delivery of DNA into regenerable cells and plant regeneration through in vitro tissue culture. Two methods are commonly used to deliver the DNA: T-DNA transfer using Agrobacterium tumefaciens or related bacteria and direct introduction of DNA via particle bombardment. It will be apparent to the skilled person that the particular choice of a transformation system to introduce a nucleic acid construct into plant cells is not essential to or a limitation of the present disclosure, provided it achieves an acceptable level of nucleic acid transfer.

Agrobacterium-mediated transformation of cannabis may be performed by methods known in the art. Any Agrobacterium strain with sufficient virulence may be used. Bacteria related to Agrobacterium may also be used. The DNA that is transferred (T-DNA) from the Agrobacterium to the recipient plant cells is comprised in a gene editing construct (i.e., chimeric plasmid) that contains one or two border regions of a T-DNA region of a wild-type Ti plasmid flanking the nucleic acid to be transferred. The genetic construct may contain two or more T-DNAs, for example, where one T-DNA contains the gene of interest and a second T-DNA contains a selectable marker gene, providing for independent insertion of the two T-DNAs and possible segregation of the selectable marker gene away from the transgene of interest.

In an embodiment, the regenerable plant cell is transformed with the gene editing construct using Agrobacterium tumefaciens, or a related bacteria.

In another embodiment, the regenerable plant cell is transformed with the gene editing construct using Agrobacterium tumefaciens strain EHA105. In another embodiment, the regenerable plant cell is transformed with the gene editing construct using the Agrobacterium tumefaciens strain LBA4404. In yet another embodiment, the regenerable plant cell is transformed with the gene editing construct using the Agrobacterium tumefaciens strain GV3101.

Transformed plants can be produced by introducing a gene editing construct described elsewhere herein into a recipient cell and growing a new plant that comprises and expresses the polynucleotide encoded by the gene editing construct, thereby modulating gene expression in the new plant. The process of growing a new plant from a transformed cell, which is in cell culture, is referred to herein as “regeneration”.

In an embodiment, the germinated plant tissue is selected from the group consisting of embryogenic cotyledons, primordial root and radicle of mature embryos.

The term “transgenic plant” as used herein refer to a plant that contains a genetic construct (“transgene”) not found in a wild-type plant of the same species, variety or cultivar. That is, transgenic plants (transformed plants) contain genetic material that they did not contain prior to the transformation. A “transgene” as referred to herein has the normal meaning in the art of biotechnology and refers to a genetic sequence, which has been produced or altered by recombinant DNA or RNA technology. If present in a plant cell, the transgene had been introduced into the plant cell or a progenitor cell by a human. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognises. The genetic material is typically stably integrated into the genome of the plant. The introduced genetic material may comprise sequences that naturally occur in the same species but in a rearranged order or in a different arrangement of elements, for example an antisense sequence or a sequence expressing an inhibitory double-stranded RNA. Plants containing such sequences are included herein in “transgenic plants”. Transgenic plants as defined herein include all progeny of an initial transformed and regenerated plant (TO plant) which has been genetically modified using recombinant techniques, where the progeny comprise the transgene. Such progeny may be obtained by self-fertilisation of the primary transgenic plant or by crossing such plants with another plant of the same species. In an embodiment, the transgenic plants are homozygous for each and every gene that has been introduced (transgene) so that their progeny do not segregate for the desired phenotype. Transgenic plant parts include all parts and cells of said plants, which comprise the transgene such as, for example, seeds, cultured tissues, callus and protoplasts.

A “non-transgenic plant”, preferably a non-transgenic cannabis plant, is one that has not been genetically modified by the introduction of genetic material by recombinant DNA techniques. The presence in a plant or seed of deletions of part of a gene as generated by site-specific endonucleases such as ZFN, TAL effectors of CRISPR type nucleases, followed by non-homologous end-joining repair in the plant cell, and progeny thereof are included herein as “non-transgenic”.

In an aspect disclosed herein, there is provided a plant comprising the transformed plant cell disclosed herein.

In an embodiment, the plants comprising the transformed plant cell disclosed herein are plants of the genus Cannabis. In another embodiment, the plants comprising the transformed plant cell disclosed herein are Cannabis sativa plants.

In a preferred embodiment, the plants comprising the transformed plant cell disclosed herein are Cannabis sativa plants with modified expression of one or more cannabinoid biosynthesis genes. The person skilled in the art would understand that modifying the expression of one or more cannabinoid biosynthesis genes may result in a Cannabis sativa plant that can produce cannabinoids at optimised levels for medicinal applications.

In another aspect disclosed herein, there is provided a regenerable plant cell produced according to the methods disclosed herein.

### Kits

The DNA-recognition moiety and gene editing constructs of the present disclosure may also be provided in a kit. The kit may comprise additional components to assist in performing the methods as described herein, such as administration devices(s), excipient(s), and/or diluent(s). The kits may also include containers for housing the various components and instructions for using the kit components in such methods.

Those skilled in the art will appreciate that the invention described herein is susceptible to variations and modifications other than those specifically described. It is to be understood that the invention includes all such variations and modifications that fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### Example 1— Design for Genome Editing for Altered Cannabinoid Profile

Genome assembly of a female Cannabis plant (“C1”) was performed by preparing Single Molecule, Real Time (SMRT) bell libraries from extracted DNA as per the manufacturer's recommendations (Pacific Biosciences of California, Inc., Menlo Park, Calif., US). Generated SMRT bell templates were sequenced using PacBio (Pacific Biosciences of California, Inc., Menlo Park, Calif., US) Sequel as per the manufacturer's recommendations. Raw reads were error corrected and assembled using SMRT Link's Hierarchical Genome Assembly Process (HGAP4).

Sequences (i.e., cannabinoid biosynthesis genes) from the C1 genome are shown in SEQ ID NOs: 164-198.

The CBDrx genome was obtained from The European Nucleotide Archive (PRJEB29284) (haps://www.ebi.ac.uk/ena/data/view/PRJEB29284). PK and Finola genome assemblies were obtained from the NCBI BioProject database (PRJNA73819) (https://www.ncbi.nlm.nih.gov/bioproject/?term=PRJNA73819).

Cannabinoid biosynthesis genes were accessed from a variety of sources and public databases (Table 3). Sequences were downloaded and used as a query for BLAST analysis against the genome assembly with an e-value threshold set at <10-10. Identified scaffold regions of interest from the reference genome were annotated and visualised using FGENESH (Solovyev et al., 2006, Genome Biology, 7(1): S10) and MEGANTE (Numa and Itoh, 2013, Plant and Cell Physiology, 55(1): e2-e2).

SNP discovery was performed, and five hundred and thirty-four whole genomes were re-sequenced on a HiSeq3000 instrument at varying depths. The resulting sequence data was reference aligned to the genome assembly of C1, using the BWA MEM algorithm (Li, 2013, ArXiv Preprint ArXiv: 1303.3997). Variants were identified using samtools (Li et al., 2009, Bioinformatics, 25(16): 2078-79) and a bed file with scaffold regions of interest matching to gene sequences of cannabinoid biosynthesis genes was created (see, e.g., variants comprised in any one of the sequences of SEQ ID NOs: 199-233). Alignments were sorted and used for variant calling with an adjusted mapping quality (−C 50) and minimum read depth of 5 to generate a consensus sequence.

Presence of an allele, or extra copies of a gene, were determined based on genomic nucleotide multiple sequence alignments using MUSCLE (Edgar, 2004, Nucleic Acids Research, 32(5): 1792-97). Sequences of similar length with alignment similarity between 80-98%, which produced identical translated proteins, were determined as alleles. Where large variation existed between genomic nucleotide sequence length and content, or where nucleotide sequences were <1000 bp, predicted mRNA sequences were used from FGENESH for alignment. Alleles were determined if similarity equaled>98%. Extra copies of genes were determined if similarity were <98%.

CHOPCHOP (Labun et al., 2016, Nucleic Acids Research, 44(W1): W272-76), CRISPR MultiTargeter (Prykhozhij et al., 2015, PLoS One, 10(3): e0119372), Crispor (Haeussler et al., 2016, Genome Biology, 17(1): 148) and ZiFit (Hwang et al., 2013, Nature Biotechnology, 31(3): 227) were used for the selection of sgRNAs. For visual confirmation of SNP avoidance, sgRNAs were manually aligned to C1 and consensus sequences using Sequencher (Gene Codes Corporation).

To locate all the genes involved in cannabinoid biosynthesis, query references were downloaded from publicly available databases (Table 3) and BLAST analyses was performed against the C1 genome assembly. All genes in the MEP, GPP, Hexanoate and Cannabinoid pathway were identified (Table 3). Two versions of 1-deoxy-D-xylulose 6-phosphate synthase (DXS) were identified in the MEP pathway, with single copies of 1-deoxy-D-xylulose 5-phosphate reductoisomerase (DXR), 4-diphosphocytidyl-2C-methyl-D-erythritol synthase (MCT), 4-diphosphocytidyl-2-C-methyl-D-erythritol kinase (CMK), 2C-methyl-D-erythritol 2,4-cyclodiphosphate synthase (MDS), 4-hydroxy-3-methylbut-2-en-1-yl diphosphate synthase (HDS) and 1-hydroxy-2-methyl-2-(E)-butenyl 4-diphosphate reductase (HDR). Single genes of isopentenyl diphosphate isomerase (IPP/IPI), geranyl pyrophosphate synthase (GPP), small and large subunits, were identified in the GPP pathway. In the hexanoate pathway, four copies of fatty-acid desaturase (FAD2) were identified using the Purple Kush (PK) desaturase gene sequence as the query and all are believed to be involved in cannabinoid biosynthesis. Lipoxygenase (LOX) and hydroperoxide lyase (HPL) were identified using the associated PK gene sequences as the queries. Acyl-activating enzyme (AAE1) was found using previously published sequences (Table 3) amongst the AAE superfamily, containing 15 AAE homologs. In the cannabinoid pathway a single copy of olivetol synthase (OLS) and three copies of olivetolic acid cyclase (OAC) were found. Two complete CBDAS genes were identified with seven closely related homologs. A single, complete copy of cannabichromenic acid synthase (CBCAS) was identified with two closely related homologs, and a single copy of THCAS was identified.

### Pan-Genome Comparison

Within the publicly available cannabis genomes, the assembled gene set was then used to query gene copy number and identify potential homologs. Differences exist between the datasets in terms of gene copy number due to the resolution of the sequence data, genetic mapping, scaffolding technologies and natural variation in different genomes. Variations in gene presence and copy number, using the assembled reference gene list, exist for DXS1, DXS2, DXR, IPP/IPI, GPP_SSU, FAD2, AAE], OLS, OAC, CBDAS, THCAS and CBCAS (Table 3). Within the Finola genome, DXS1, DXS2, GPP_SSU and AAE] were not identified, with copy number variation observed for FAD2, OLS and OAC when compared to C1. Within the CBDrx genome, no copy of IPP/IDI was identified, while copy number variations were identified for FAD2 and synthase genes compared to C1. The updated PK genome had at least one copy of each gene, with variations in copy numbering existing for DXR, FAD2, OLS and OAC compared to C1.

### Analysis of Single Nucleotide Polymorphisms (SNPs) and Informed Guide Design

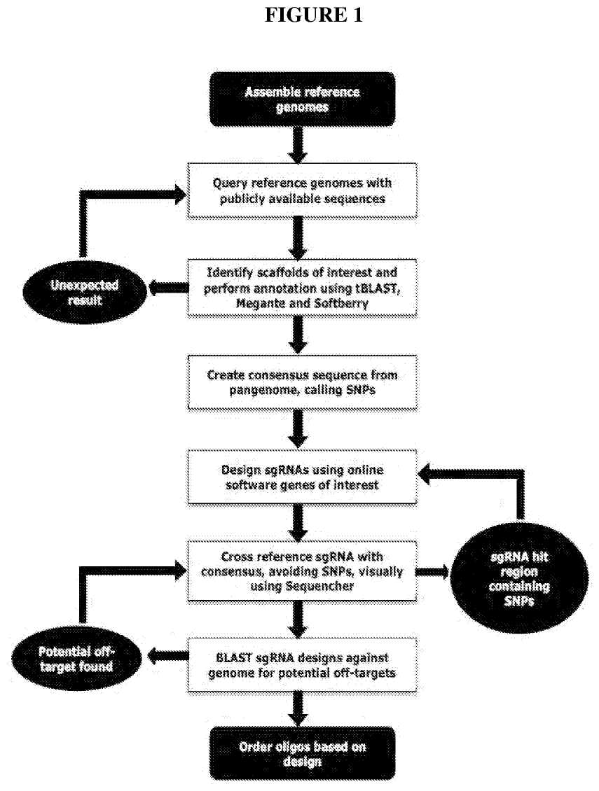

To assess gene variation, an established resource of SNP locations were overlaid onto the identified genes integral to the cannabinoid biosynthesis (Table 4). With the exception of FAD2, which belongs to a large, diverse family of desaturases and CBDAS#a, a homolog of CBDAS, the cannabinoid biosynthesis genes contain relatively conserved total variations in their sequences (Table 5). Each consensus sequence, containing SNP locations was then used for intelligent guide designs to avoid all known nucleotide variations, creating universal guides, which can be broadly used on any plant genotype within the species, and in the instance of highly similar gene sequences, unique guides designed to target only a specific gene of interest (FIG. 1). Sequences from the reference genome were entered into the online design tools CHOPCHOP, CRISPR MultiTargeter, Crispor and ZiFit to generate guides based on their preferred scoring matrixes. These guides were then manually compared and visually assembled using Sequencher. Taking the highest-ranking scores from each online tool, which predict off-targets and greatest binding affinity, a total of 183 sgRNAs were designed targeting every gene in the combined pathways (Table 5). Within these guides, MultiTargeter was used with multiple gene copies or when alleles had highly homologous sequences to design 32 universal guides targeting both alleles, which varied in the exons, for CMK, HDS, LOX, OAC, THCAS, CBCAS and CBDAS. All guides were re-BLAST against reference genome for detection of off-site targeting, with results confirming no complete 20-nt sgRNA had potential off-targets.

Phytocannabinoids are of particular interest for their pharmacological applications in a growing number of medical conditions. Knowledge and understanding of the gene interactions and their relationship to final cannabinoid concentration can facilitate improved cannabis strains with desired novel cannabinoid levels. Creating a pangenome consensus of each gene in the contributing pathways allows for genomically informed decisions, based on known SNP location and frequency as well as presence absence variations (PAV), for crop improvement by means of genome editing. Using publicly available sequence information (Table 3), at least one full-length transcript for all genes involved in cannabinoid biosynthesis were found. Two DXS genes were also identified. Single copies of DXR, HDR and IPI/IPP were identified in the C1 genome. Fatty acid desaturase enzymes belong to two large multifunctional classes, either membrane bound, or soluble. The desaturase of interest in cannabinoid production is involved in the hexanoate pathway, leading to the production of hexanoyl-CoA, the first precursor in the cannabinoid pathway. Despite the complexity of the number of FAD2 gene sequences, four copies of this gene were identified. THC-rich PK cultivar was shown to have two copies of OLS and OAC, whereas CBD-rich cultivar, CBDrx, had just one copy of each. The C1 cultivar, with relatively equal cannabinoid levels, was shown to contain a single copy of OLS and 2 copies of OAC. Using the synthase genes from the C1 sequence as the query against CBDrx, Finola and PK genomes, the total number of synthase genes varied considerably between the cultivars. In the CBDrx genome (Grassa et al., 2018, BioRxiv, 458083, doi: https://doi.org/10.1101/458083), 13 synthase genes were reported. 11 were identified using our sequences as queries. Identification of which synthase genes were not identified is difficult due to the nested repeating nature of synthase genes around the centromere. However, variation in synthase genes is most likely due to PAV across different cultivars, which in the case of maize is common (Springer et al., 2009, PLoS Genetics, 5(11): e1000734). Total synthase gene number is not given for Finola or PK (Laverty et al., 2019, Genome Research, 29(1): 146-56), however 9 and 14 genes were found, respectively.

Within the Finola genome, 4 genes could not be identified. Both forms of DXS were not present. GPP SSU and AAE] were also not identified. AAE] was found to be the gene that synthesises hexanoyl-CoA from hexanoate supplying the cannabinoid pathway (Stout et al., 2012, The Plant Journal, 71(3): 353-65) and since Finola still produces cannabinoids, this result was considered an assembly error. GPP is a heterodimer requiring both subunits, large and small, for optimum activity. GPP activity has been previously shown to be active but at lower levels when the small subunit was inactive (Wang and Dixon, 2009, Proceedings of the National Academy of Sciences U.S.A., 106(24): 9914-19), however both subunits were still present, suggesting the absence of GPP SSU in the Finola genome is also due to assembly error. The absence of IPP/IPI in the CBDrx genome is also strongly suggested to be due to assembly error, since previous studies on Arabidopsis double mutant knockdown of IPP/IPI produced dwarfism and male sterility (Okada et al., 2008, Plant and Cell Physiology, 49(4): 604-16).

The SNP location resource revealed some genes are more highly conserved than others (Table 5). Comparative analysis of SNPs present in genes of variable copy number in C1, CBDrx, Finola and PK genomes was performed (excluding results of no gene presence). Through multiple sequence alignments of coding sequences, it was observed that the presence of SNP's occurred in the extra gene copy where the presence of homozygous alleles exist, suggesting that either sequencing error has occurred, or in fact there is an extra copy of the gene and a set of alleles. Within the C1 genome, OAC produced three hits with two sequences determined as alleles with an extra copy of the gene existing. When sequences were aligned, SNPs occurred in all sequences and when sequences were translated, nearly identical protein sequences (>99%) were produced confirming that an extra copy of the gene was present, potentially in a hemizygous condition. Within the PK genome, copy number variation was shown for OLS and OAC. Like OAC in the C1 genome, OLS produced three hits, two of which were determined to be alleles and one to be an extra copy. SNPs were identified in all three sequences when coding regions were aligned with similar results obtained from protein sequence alignment. Initial alignment of both OAC hits, in PK, found a 98.5% similarity in genomic sequences, however no gene prediction was possible on one of the sequences, possibly due to a premature stop codon from a SNP rendering this gene inactive potentially indicating that it exists as a pseudogene.

Using multiple tools for the design of sgRNA ensured that all possible guide designs could be assessed for in silico off-targeting. Each tool implemented different scoring rules based on off-targets, mismatches, efficiency score, existence of self-complimentary regions, GC content, location of guide and multiple sequence alignments (Prykhozhij et al., supra; Labun et al., supra). Due to the absence of a fully developed pan-genome for analysis by these tools, the use of multiple tools was necessary. The presence of a PAM site is necessary for sgRNA binding and while these tools scanned the gene sequence for the PAM sites, the results obtained varied between the online tools. Visualisation of guides was clear using CHOPCHOP compared to the other tools and regularly provided the best guide designs. However, when highly homologous sequences were used MultiTargeter was able to perform sequence alignments and produce unique guides for each sequence, a feature not possible within the other tools. Designing the guides for the unique synthases were first run using MultiTargeter and further verified using CHOPCHOP for visualisation. Guides designed were targeted to the earliest possible exon for maximum likelihood of a frame shift mutation. The error prone nature of NHEJ often occurs with small deletions, or insertions, occurring at the DSB leading to protein misfolding and thus production of a knock out gene. Each identified gene, with accompanying allele where applicable, were analysed and sgRNAs were designed to be either universal, inactivating both alleles, or if sequence heterozygosity exists, specific guides were designed (Table 5). Using genome editing, sequence homogeneity between synthase genes could potentially lead to off-target editing, with targets suggested to have at least several nucleotides different for discrimination (Soyars et al., 2018, Plant and Cell Physiology, 59(8): 1608-20). Where possible, each synthase gene, and accompanying homologs, had universal and specific guides designed that could be used regardless of cultivar chosen as the target.

The reported sequence similarity between THCAS, CBDAS and CBCAS, of up to 95% (Laverty et al., supra) requires precise, intelligent design, using multiple online tools and a large consensus population to improve the likelihood of correct gene knockout. Off-targeting predictions, given by sgRNA online tools, currently use the previously fragmented genome of PK (van Bakel et al., supra). To circumvent this, each sgRNA was used as a query to BLAST against the C1 genome for potential off-targets. From the BLAST results no sgRNA had an unexpected sequence match elsewhere in the genome.

TABLE 3
Source of gene query/NCBI accession number and gene
copy and homolog number for available genomes

| | NCBI Accession | SEQ | | | | |
|---|---|---|---|---|---|---|
| | Number/Source | ID | Copy number/homologs | | | |
| Gene | of Query | NO: | C1 | CBDrx | Finola | PK V2 |
| DXS1 | KY014576.1 | 1 | 1 | 1 | — | 1 |
| DXS2 | KY014577.1 | 2 | 1 | 1 | — | 1 |
| DXR | KY014568 | 3 | 1 | 1 | 1 | 2 |
| MCT | KY014578 | 4 | 1 | 1 | 1 | 1 |
| CMK | KY014575 | 5 | 1 | 1 | 1 | 1 |
| MDS | HQ734721.1 | 6 | 1 | 1 | 1 | 1 |
| HDS | KY014570.1 | 7 | 1 | 1 | 1 | 1 |
| HDR | KY014579.1 | 8 | 1 | 1 | 1 | 1 |
| IPP/IPI | KY014569.1 | 9 | 1 | — | 1 | 1 |
| GPP | KY014573.1 | 10 | 1 | 1 | 1 | 1 |
| LSU | | | | | | |
| GPP | KY014567.1 | 11 | 1 | 1 | — | 1 |
| SSU | | | | | | |
| FAD2 | PK genome, | 12 | 4 | 5 | 5 | 3 |
| | scaffold71447:2,827- | | | | | |
| | 3,852 | | | | | |
| LOX | PK genome, | 13 | 1 | 1 | 1 | 1 |
| | scaffold53609:3,286- | | | | | |
| | 7,284 | | | | | |
| HPL | PK genome, | 14 | 1 | 1 | 1 | 1 |
| | scaffold14797:30,184- | | | | | |
| | 30,623 | | | | | |
| AAE1 | JN717233 | 15 | 1 | 1 | — | 1 |
| OLS | EU551162.1 | 16 | 1 | 1 | 1 | 2 |
| OAC | JN679224.1 | 17 | 2 | 1 | 1 | 2 |
| GOT | Publication number: | 18 | 1 | 1 | 1 | 1 |
| | US20120144523A1 | | | | | |
| CBDAS | AB292682 | 19 | 9¹ | 11 total | 9 total | 14 total |
| THCAS | AB057805 | 20 | 1 | | | |
| CBCAS | Publication number: | 21 | 3² | | | |
| | WO/2015/196275 | | | | | |

¹2 genes and 7 homologs;
²1 gene and 2 homologs.

TABLE 4
Variance amongst genes involved
in cannabinoid biosynthesis in C1

| Gene | Length | #SNPs | % Total |
|---|---|---|---|
| DXS1 | 373* | 6 | 1.6 |
| DXS2 | 2892 | 71 | 2.5 |
| DXR | 3689 | 68 | 1.8 |
| MCT | 4242 | 155 | 3.7 |
| CMK | 4031 | 103 | 2.6 |
| MDS | 1946 | 70 | 3.6 |
| HDS | 5383 | 211 | 3.9 |
| HDR | 2309 | 76 | 3.3 |
| IPP/IPI | 2921 | 50 | 1.7 |
| GPP_LSU | 1281 | 31 | 2.4 |
| GPP_SSU | 1061 | 19 | 1.8 |
| FAD2#1 | 1123 | 57 | 5.1 |
| FAD2#2 | 1085 | 52 | 4.8 |
| FAD2#3 | 1091 | 53 | 4.9 |
| FAD2#4 | 1084 | 25 | 2.3 |
| LOX | 4162 | 133 | 3.2 |
| HPL | 7201 | 200 | 2.8 |
| AAE1 | 6688 | 220 | 3.3 |
| OLS | 1418 | 35 | 2.5 |
| OAC | 692 | 17 | 2.5 |
| OAC#2 | 548 | 15 | 2.7 |
| GOT | 7350 | 264 | 3.6 |
| CBCAS | 1506 | 2 | 0.1 |
| CBCAS-like#a | 1506 | 2 | 0.1 |
| CBCAS-like#b | 1013 | 5 | 0.5 |
| CBDAS#a | 538 | 40 | 7.4 |
| CBDAS#b | 919 | 46 | 5.0 |
| CBDAS-like#a | 1362 | 14 | 1.0 |
| CBDAS-like#b | 1394 | 13 | 1.0 |
| CBDAS-like#c | 1326 | 3 | 0.2 |
| CBDAS-like#d | 1326 | 0 | 0 |
| CBDAS-like#e | 1152 | 14 | 0.2 |
| CBDAS-like#f | 1506 | 24 | 1.6 |
| CBDAS-like#g | 463 | 9 | 2.0 |
| THCAS | 1506 | 37 | 2.5 |

*cds only

TABLE 5
Targeted sgRNA sequence design for the CBDAS/THCAS pathway in
Cannabis sativa

| | | | | Common sgRNA |
|---|---|---|---|---|
| | | | SEQ ID | for genes/ alleles/ |
| sgRNA | Gene | Sequence | NO: | homologs |
| 1 | DXS1 | AAGGTACCCGGCATTATTCA | 22 | |
| 2 | DXS1 | GCTGTTGGAAGGGATCTTAA | 23 | |
| 3 | DXS1 | CATGTCGGAATCAAGGTACC | 24 | |
| 4 | DXS1 | ATAAGCTTGACCTGCTGTCA | 25 | |
| 5 | DXS2 | CATACAGTATCAAAGACAGG | 26 | |
| 6 | DXS2 | TGGGGCCATGACTGCAGGAA | 27 | |
| 7 | DXS2 | AGAAAGACTTCTGGTCTAGC | 28 | |
| 8 | DXS2 | TACCCGCATAAGATTCTTAC | 29 | |
| 9 | DXS2 | CCGGTGGATGGACATAATGT | 30 | |
| 10 | DXS2 | GAGGAATGATAAGTGCTTCT | 31 | |
| 11 | DXS2 | TCAGGTGCTACTTCAGCTGG | 32 | |
| 12 | DXS2 | ATACACGCAGCAATGGGTGG | 33 | |
| 13 | DXR | ACACAGCTTAGGGAAGCGGG | 34 | |
| 14 | DXR | CTGGACACAGCTTAGGGAAG | 35 | |
| 15 | DXR | GTGGAGCCTAGAATTGAAAC | 36 | |
| 16 | DXR | AGAGTAGTGGGACTTGCAGC | 37 | |
| 17 | DXR | TTCGGCAAGAAGGGTTACAT | 38 | |
| 18 | MCT | GTGAACTGATTTTGCGGGGT | 39 | |
| 19 | MCT | TGGGTGTTTAGTCCATGAGG | 40 | |
| 20 | MCT | TCATGTGAACTGATTTTGCG | 41 | |
| 21 | CMK | ACTGGAGCAGGGCTAGGTGG | 42 | Multi-target |
| 22 | CMK | AGAAAGTGCCCACTGGAGCA | 43 | Multi-target |
| 23 | CMK | ACCTCTGTTCCTAAAAGGAT | 44 | Multi-target |
| 24 | CMK | GCCTTCATCTCTCAAAATTG | 45 | |
| 25 | CMK | CCTCTGTTCCTAAAAGGATT | 46 | |
| 26 | MDS | CGCTGAGCAAGCTCCGTCTG | 47 | |
| 27 | MDS | GACGGCCATGGCGGCGGCGA | 48 | |
| 28 | MDS | GTCGCGGCGGCCGATACAGA | 49 | |
| 29 | MDS | GGAGTAGCAGATACCTCAGA | 50 | |
| 30 | HDS | CCTTACACAAAACTGTTAGG | 51 | |
| 31 | HDS | GAGGCTTTCTGATTTAAAGA | 52 | |
| 32 | HDS | CTAAAAGTTCTGATTTTGTG | 53 | |
| 33 | HDS | GATGACGACAAATGATACCA | 54 | |
| 34 | HDS | GAAGTGGATAGTCCCAGCCC | 55 | |
| 35 | HDS | ACTCACTGAACCGCCCGAGG | 56 | |
| 36 | HDS | CTGGGACTATCCACTTCACT | 57 | Multi-target |
| 37 | HDS | CACTTGGGAGTTACTGAAGC | 58 | Multi-target |
| 38 | IPP/IPI | GGCAATGTAGAGCTTGGCAG | 59 | Multi-target |
| 39 | IPP/IPI | ATGGGAGACTCTGCCGACGC | 60 | Multi-target |
| 40 | IPP/IPI | CTTGTCTCCTCTCACCGCTA | 61 | |
| 41 | IPP/IPI | CCTTGTCTCCTCTCACCGCT | 62 | |
| 42 | IPP/IPI | TTTCTGCAGATGCATTCTAG | 63 | |
| 43 | GPP_LSU | TGTTCCATGTTCAACCAAGG | 64 | |
| 44 | GPP_LSU | GGCGAGGAGTGAGTAACGCA | 65 | |
| 45 | GPP_LSU | CAGGCTGAGAGACAGAGCAT | 66 | |
| 46 | GPP_LSU | TCTACCTCCTTGGTTGAACA | 67 | |
| 47 | GPP_SSU | GGCATCTACATCGGCTGTTG | 68 | |
| 48 | GPP_SSU | TTTAGCACCGTCATTGTGCG | 69 | |
| 49 | GPP_SSU | AGCACCGTCATTGTGCGTGG | 70 | |
| 50 | LOX | CTAATCCTTGACTCAGATAA | 71 | Multi-target |
| 51 | LOX | ATTAAGACTTGTCTGAGTAT | 72 | Multi-target |
| 52 | LOX | TAACCAATTCAATGCAGCAA | 73 | Multi-target |
| 53 | LOX | ACAAGAAAGACGCACCTGGG | 74 | Multi-target |
| 54 | LOX | TATGAGAAGACCGTCGTTGG | 75 | Multi-target |
| 55 | LOX | TGTTATGCCGTCAGAAGAGA | 76 | Multi-target |
| 56 | LOX | TTGCTGCATTGAATTGGTTA | 77 | Multi-target |
| 57 | HPL | TTCCTGGTCAAGTGTCTAAT | 78 | |
| 58 | HPL | ACAAAGCTCGAAGATATGGG | 79 | |
| 59 | HPL | GCCACAAATAGAGCATCGAC | 80 | |
| 60 | HPL | AAGGATCCCAATCTTGATAG | 81 | |
| 61 | HPL | GCGAATTGGAGCGGAACAGG | 82 | |
| 62 | HPL | CGATCCCGGGAAGCTACGGA | 83 | |
| 63 | HPL | GTAGTCTAACCGGTCCGAGA | 84 | |
| 64 | HPL | GCCCAGTGTCAATTACACTG | 85 | |
| 65 | HPL | TACTGGGGCCCATCTCGGAC | 86 | |
| 66 | HPL | GGACCCCAGACCCCAGACAC | 87 | |
| 67 | HPL | CGGACCCAGACTCCAGACCT | 88 | |
| 68 | OLS | GTTTCCCGACTACTACTTTC | 89 | |
| 69 | OLS | TCATCTTCGTGCTGAGGGTC | 90 | |
| 70 | OLS | GTGCAAAGGCCATCAAAGAA | 91 | |
| 71 | OLS | GTCTGCACCGGGCATGTCAG | 92 | |
| 72 | OAC | TTACCAGTATACATCTTTCA | 93 | |
| 73 | OAC | AGTATACATCTTTCATGGCT | 94 | |
| 74 | OAC | CAGTATACATCTTTCATGGC | 95 | Multi-target |
| 75 | OAC | TGAAATCACAGAAGCCCAAA | 96 | Multi-target |
| 76 | OAC | ATTATTCATCCTGCCCATGT | 97 | |
| 77 | OAC | ATTGTTCATCCGGCCCATGT | 98 | |
| 78 | GOT | AGCATAATTGTGGCCCTAAC | 99 | |
| 79 | GOT | GCATAATTGTGGCCCTAACT | 100 | |
| 80 | FAD2#1 | AGAAACAAAATGGGAGCCGG | 101 | |
| 81 | FAD2#1 | TTTCAGTGACCACCAATGGG | 102 | |
| 82 | FAD2#1 | CCACTTTATTGGATCTTCCA | 103 | |
| 83 | FAD2#2 | GAAACAAAAATGGGAGCCGG | 104 | |
| 84 | FAD2#2 | ATGGCTCTACAAACACTACT | 105 | |
| 85 | FAD2#1 | ATTGGTGGTCACTGAAAGAG | 106 | |
| 86 | FAD2#2 | CTAGGGAGAGTCCTAACACT | 107 | |
| 87 | FAD2#2 | AGTGTTAGGACTCTCCCTAG | 108 | |
| 88 | FAD2#2 | GGAGTCAATGGTGAAAACAG | 109 | |
| 89 | FAD2#3 | AGAGAGCGTTGGAAGCAATG | 110 | |
| 90 | FAD2#3 | CTACAAATGGATTGATGACA | ill | |
| 91 | FAD2#4 | CCTTGGGAGATCCAATAAAG | 112 | |
| 92 | FAD2#4 | ATTTCGCTTAGCGTGAATGG | 113 | |
| 93 | FAD2#4 | AGCCTTGGGAGATCCAATAA | 114 | |
| 94 | FAD2#4 | GCTTAGCGTGAATGGTGGTT | 115 | |
| 95 | AAE1 | GGCGCACTTTTGGAGAAGCG | 116 | |
| 96 | AAEI | GCCAATGCATGTGGATGCTG | 117 | |
| 97 | AAEI | ATTTTCTGTAAGAAACCCTG | 118 | |
| 98 | AAEI | GGTAGTGAATGGCTTCCAGG | 119 | |
| 99 | THCAS | GAAGGAGTGACAATAACGAG | 120 | Multi-target |
| 100 | THCAS | AGGACATACCCTCAGCATCA | 121 | |
| 101 | THCAS | TCAAGTCTACTACAACAAAT | 122 | |
| 102 | THCAS | GACAATAACGAGTGGTTTTG | 123 | |
| 103 | CBCAS | TATTGCCCTACTGTTGGCGT | 124 | Multi-target |
| 104 | CBCAS | TCAAGTCTACTATAGCAAAT | 125 | |
| 105 | CBCAS | TATTCATAGCCAAACTGCGT | 126 | Multi-target |
| 106 | CBCAS | AGACTTGAGAAACATGCATA | 127 | |
| 107 | CBCAS-like#a | ATATTCATAGCCAAACTGCG | 128 | |
| 108 | CBCAS-like#b | GTCCACCTACGCCAACAGTA | 129 | |
| 109 | CBDAS#a | GTCCAGCTGCGCTAACAGTA | 130 | |
| 110 | CBDAS#a | TGTCCAGCTGCGCTAACAGT | 131 | |
| ill | CBDAS#a | GCAGCTGGACACTTTGGTGG | 132 | |
| 112 | CBDAS#a | TGTTCATAGCCAAATCGCAA | 133 | |
| 113 | CBDAS#b | TATAGCGGTGTTGTAAATTA | 134 | |
| 114 | CBDAS#b | GTTAGATCAGCTGGGCAGAA | 135 | |
| 115 | CBDAS#b | GGAATATTACAGATAATCAA | 136 | |
| 116 | CBDAS#b | GATACTATCATCTTCTATAG | 137 | |
| 117 | CBDAS-like#a | GCGGGTGGACACTTTAGTGG | 138 | Multi-target |
| 118 | CBDAS-like#a | TACTGCCCTACTGTTGGCGC | 139 | Multi-target |
| 119 | CBDAS-like#a | TGTCCACCCGCGCCAACAGT | 140 | |
| 120 | CBDAS-like#a | GTACTGCCCTACTGTTGGCG | 141 | |
| 121 | CBDAS-like#b | ACAGTAGGGCAGTACCCAGC | 142 | Multi-target |
| 122 | CBDAS-like#b | GATGCGAAATTATGGCCTCG | 143 | Multi-target |
| 123 | CBDAS-like#b | GCTGGGTACTGCCCTACTGT | 144 | Multi-target |
| 124 | CBDAS-like#b | GGTAAATCTAAGATTTTGTA | 145 | |
| 125 | CBDAS-like#b | TGGAACATAGAATAGTGCCT | 146 | |
| 126 | CBDAS-like#c | TTTTAGATCGAAAATCCATG | 147 | Multi-target |
| 127 | CBDAS-like#e | ACAGTAGGACAGTACCCAGC | 148 | |
| 128 | CBDAS-like#e | TGGAGCATAGAATAGTGCCT | 149 | |
| 129 | CBDAS-like#e | TCAAGTCTACTATAACAAAT | 150 | |
| 130 | CBDAS-like#f | GAGAATCTTAGTTTTCCTGC | 151 | |
| 131 | CBDAS-like#f | ACTTTGGAATCATTGCAGCG | 152 | |
| 132 | CBDAS-like#g | ACATGATTCCAGCTCGATGA | 153 | |
| 133 | CBDAS-like#g | TACATGATTCCAGCTCGATG | 154 | |
| 134 | CBDAS-like#g | GTTAGTAAAAGTAAAAACCA | 155 | |
| 135 | CBDAS-like#g | ATTTGGGGTGAAAAGTATTT | 156 | |
| 136 | CBDAS#a + b | GTGCTAGATCGAAAATCTAT | 157 | Multi-target |
| 137 | CBDAS#a + b | GATCTCTTTTGGGCTATACG | 158 | Multi-target |
| 138 | CBDAS#a + b | AGTGCTAGATCGAAAATCTA | 159 | Multi-target |
| 139 | CBDAS#a + b | GTTAGATCAGCTGGGCAGAA | 160 | Multi-target |
| 140 | CBDAS#a + b | CTAAACATAGTAGACTTTGT | 161 | Multi-target |
| 141 | CBDAS#a + b | TAAACATAGTAGACTTTGTT | 162 | Multi-target |
| 142 | CBDAS#a + b | ACAAATGCAGATTCTGGAAT | 163 | Multi-target |

### Example 2— Transient Rapid Evaluation of Genome Edit Constructs for Identification of the Optimal Efficacious Molecules

### Preparation and Sterilisation of Cannabis Explants

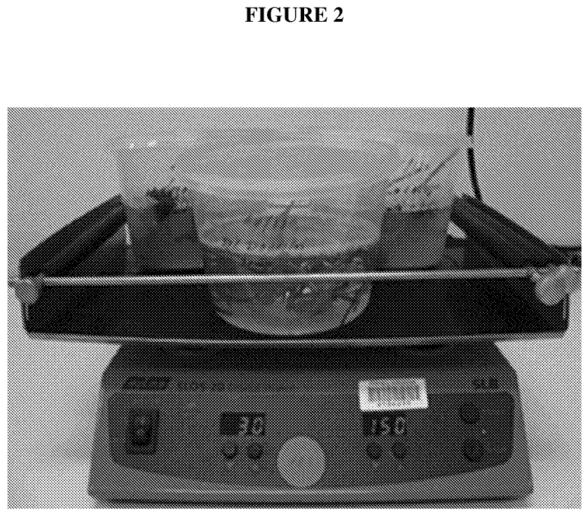

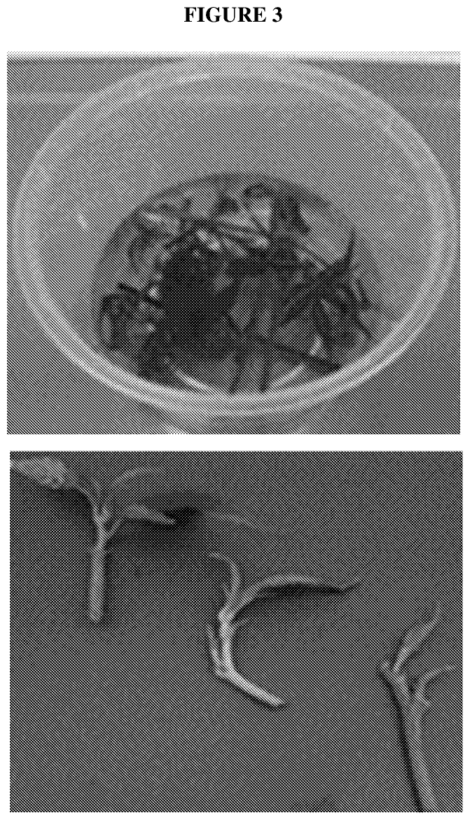

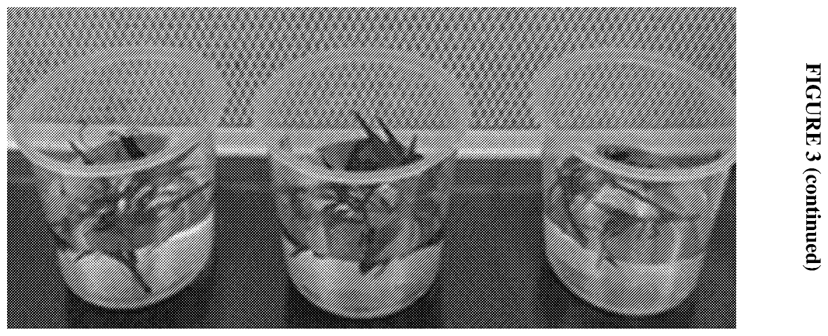

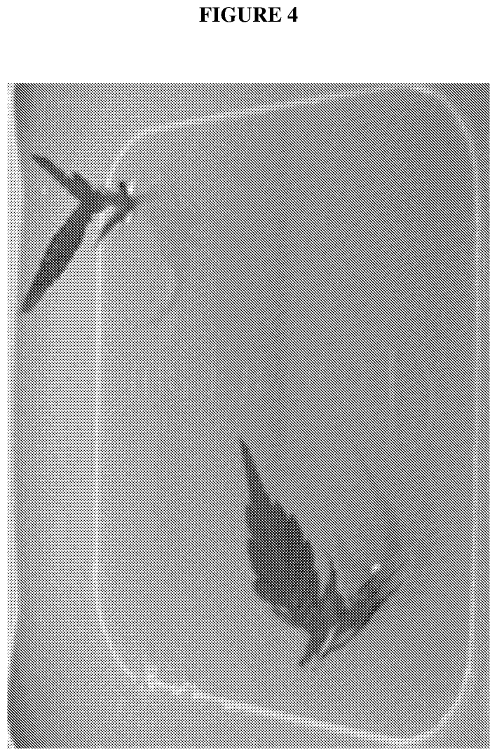

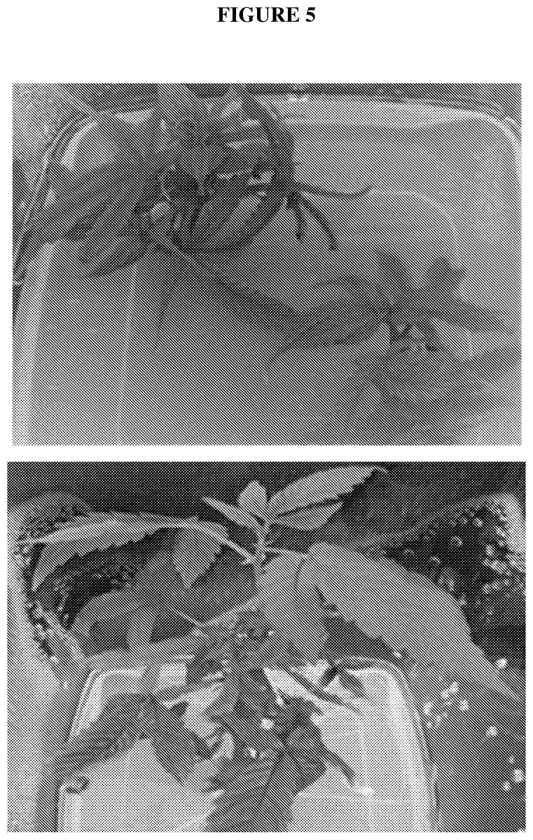

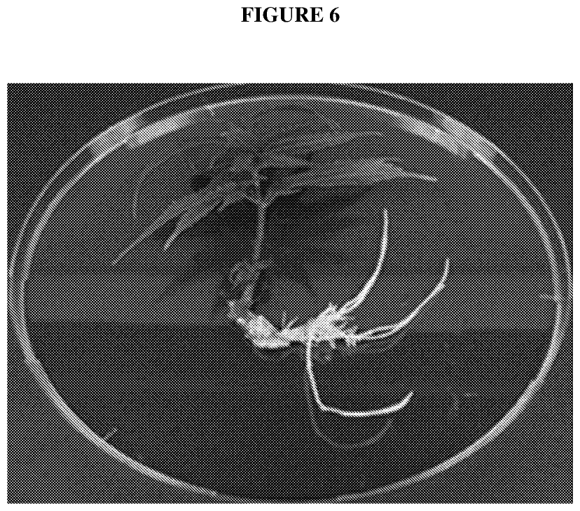

Buds were excised from the initial mother plant and then cleaned by rinsing several times under tap water. The buds were then surface sterilised by stirring in 80% Ethanol (v/v) for 1 minute. The ethanol was then decanted off from the plant tissue, and the cannabis buds rinsed with tap water for a minimum of three times, changing the water in between each rinse. The buds were then immersed in 15% Domestos® [4.75% available Chlorine m/v] for 15 min with shaking at 150 rpm/min (FIG. 2). Prior to adding the cannabis buds, several drops of Tween 20 were added to the Domestos®. The material at this point was transferred to an aseptic laminar flow cabinet, where the Domestos® was decanted away from the cannabis buds and the plant material was then rinsed repeatedly with sterile Milli-Q water, until all of the sterilising agent has been removed (FIG. 3). The removal of the agent was indicated by the lack of white foam around the buds. The cannabis buds were then retained in a final sterile Milli-Q water wash for 2-5 minutes. The apical buds of the cleaned tissue were then excised avoiding any unhealthy or necrotic material, excision midway between the shoot tip and the stem. The excised apical buds were then inspected under a stereo microscope to check for insects remaining on the explant or any other issues of poor plant health. The selected apical buds were transferred to shooting/rooting media (Table 6) and incubated for 3-4 weeks at 25±1° C./16 hr light (FIG. 4). Following every 3-4 weeks period the plants were sub-culturing onto fresh shooting/rooting media (FIGS. 5 and 6).

TABLE 6
Cannabis shooting/rooting media composition and preparation
Shooting/rooting media composition

| Composition | g/L |
|---|---|
| Murashige and Skoog Basal Medium | 2.2 gm |
| with MS Vitamins (half strength) | |
| Sucrose grade II (1%) | 10 gm |

pH conditions

| pH | Buffer/s |
|---|---|
| 5.7 | 1M NaOH/HCl |

Gelling agents

| Gelling agent | Final concentration | g/L |
|---|---|---|
| Plant Agar | 1.0% | 10 gm |

Method of sterilisation
Autoclave 121 PSI/16 min
Hormones

| Component | Final conc. | Stock | V/L |
|---|---|---|---|
| IBA | 1.0 mg/l | 1.0 mg/ml | 1000 μl |

Container details for dispensing
946 ml SteriCon ™ sterilised tub
Media Pour Details
100-120 ml/Tub
Storage requirements
Room temperature or 4° C. cold room storage

### Protoplast Isolation, Direct DNA Delivery and Screening

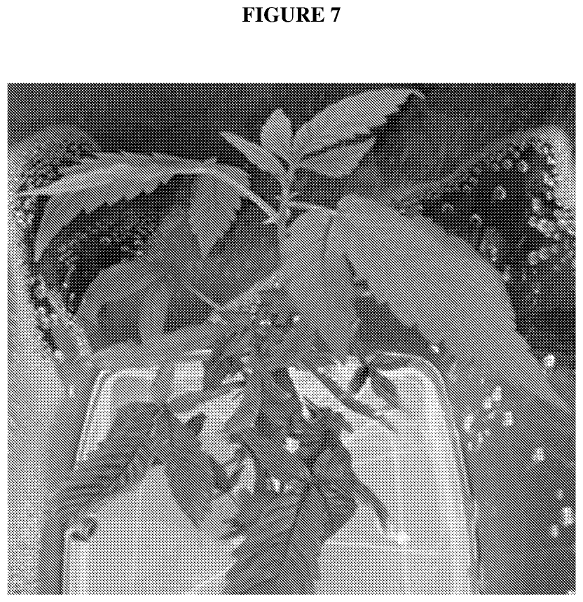

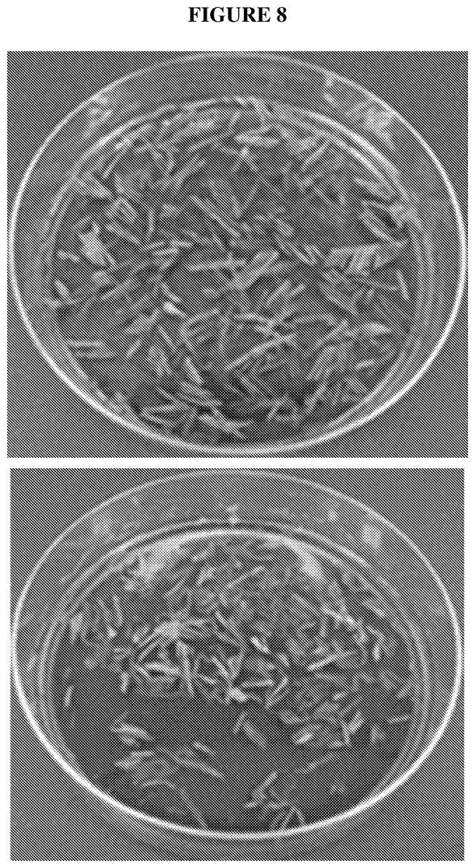

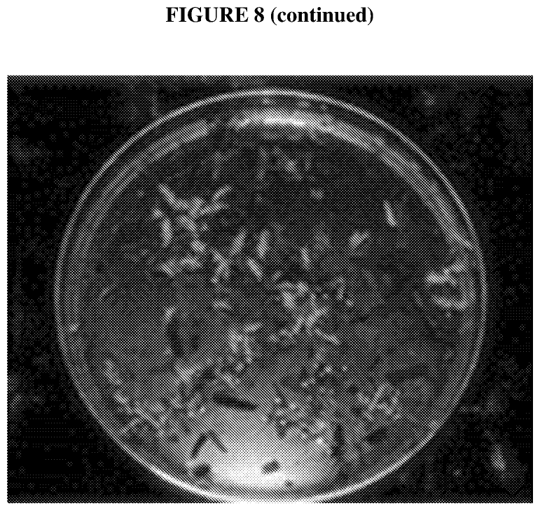

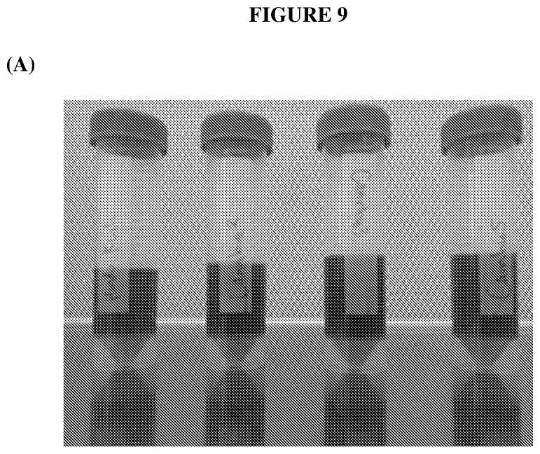

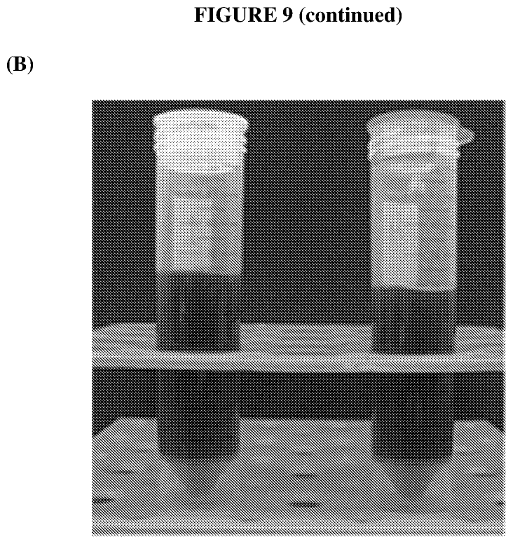

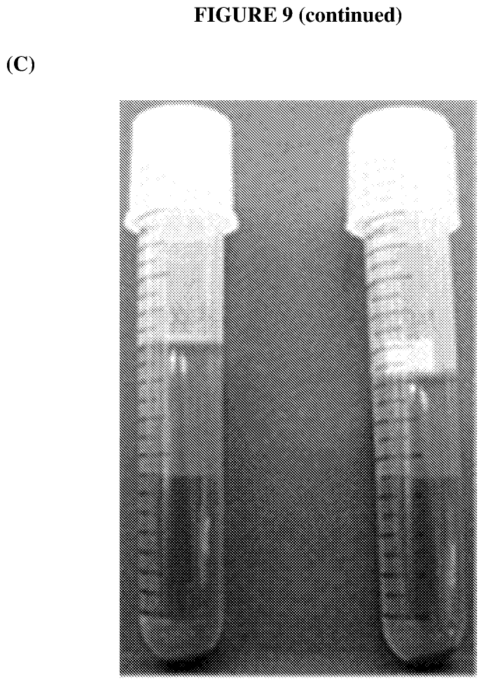

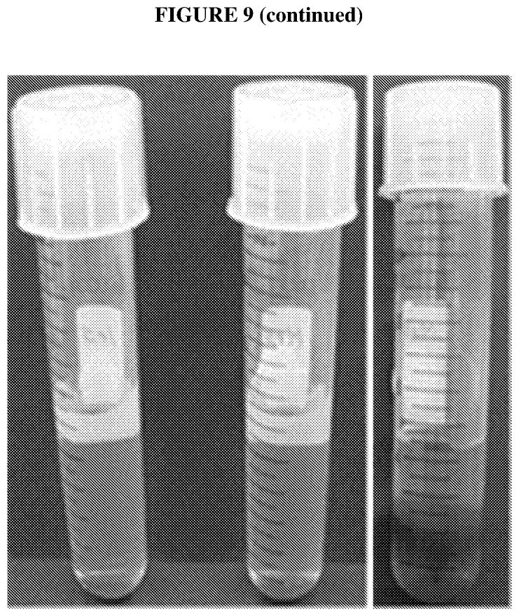

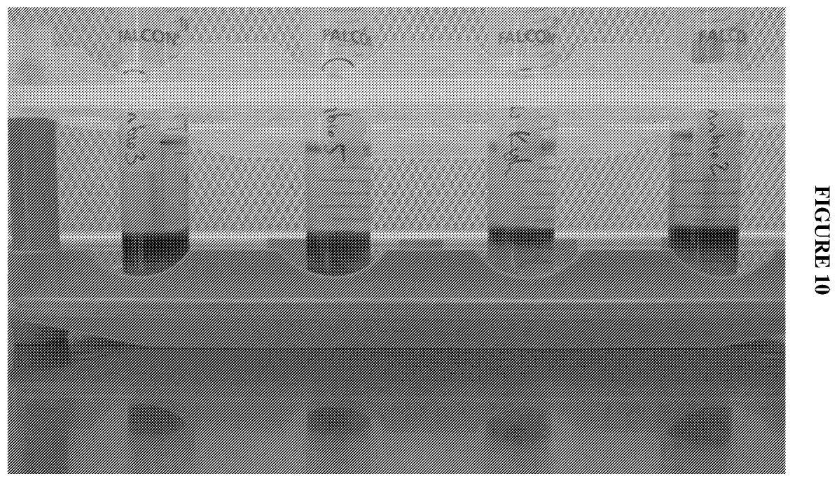

Once the in vitro plants were established and generated sufficient leaf material for protoplast isolation, leaf strips were taken. Protoplasts were isolated from well rooted, 1 month old, young leaves from a plantlet (FIG. 7) cut into 0.5-1.0 mm thin strips and incubated in digestion media (Table 7) in a petri dish containing 1-2.5% (w/v) cellulase R-10,0.2-0.5% (w/v) macerozyme 0.2% (w/v), pectolyase Y-23 (FIG. 8), pH adjusted to 5.8 and filter sterilised using a 0.22 μm filter. Leaf strips were incubated in the dark for 8-16 h at 28° C. without shaking. After digestion, the digested leaf mesophyll material was manually filtrated through 70 μm mesh filter into a 50 mL Falcon tube (FIG. 9) and centrifuged at 700 rpm for 10 min

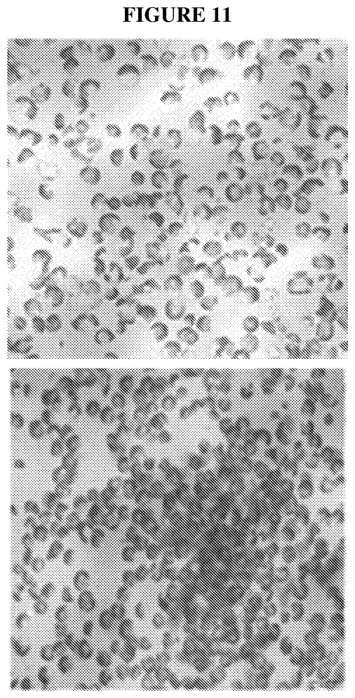

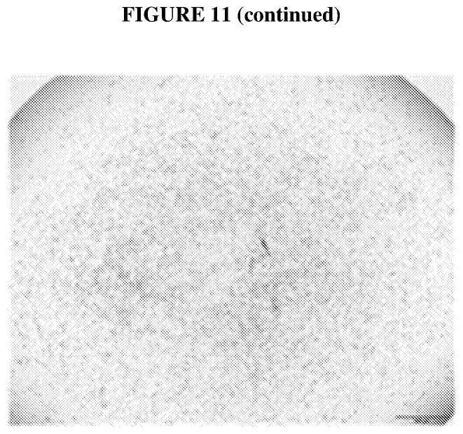

After incubation, the enzyme mixture was filtered through a sterile 70 μm cell strainer and centrifuged at 700 rpm for 10 minutes (Eppendorf Model 5910R) before decanting off the supernatant. The pellet was then resuspended in 3 ml W5 buffer (Table 8) transferred to a 14 mL round bottomed tube and 3 ml of 20% sucrose was added and the centrifugation is repeated (FIG. 9). The protoplasts were collected from the interphase of the separated liquid layers and transferred to a fresh 14 mL round bottom tube and a fresh aliquot of buffer W5 was added and the centrifugation repeated. Finally, after discarding the supernatant again the protoplasts were resuspended in 1 ml W5 buffer (Table 8), and the cells counted. Collected protoplasts were treated with 0.5 M Evans Blue (1:10, v/v), incubated for 10 min with yield and viability calculated using a haemocytometer under a light microscope (FIG. 11.). The viability of the protoplasts was calculated by (viable protoplasts/total number of protoplasts)×100%.

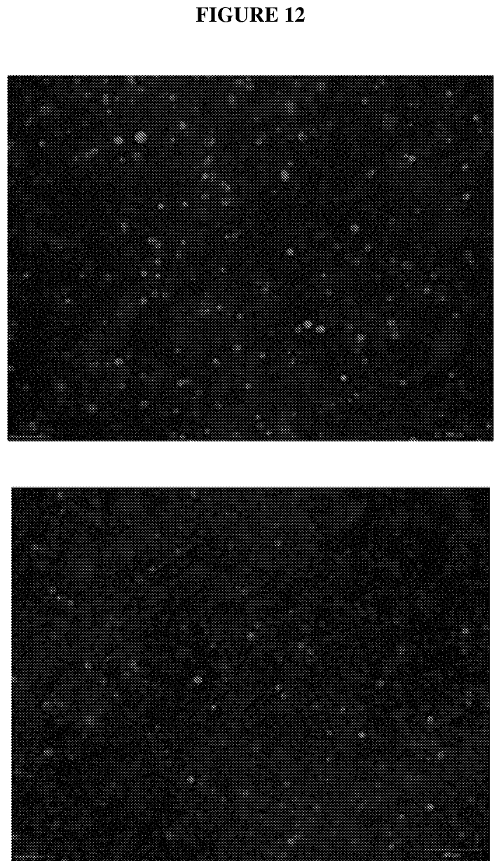

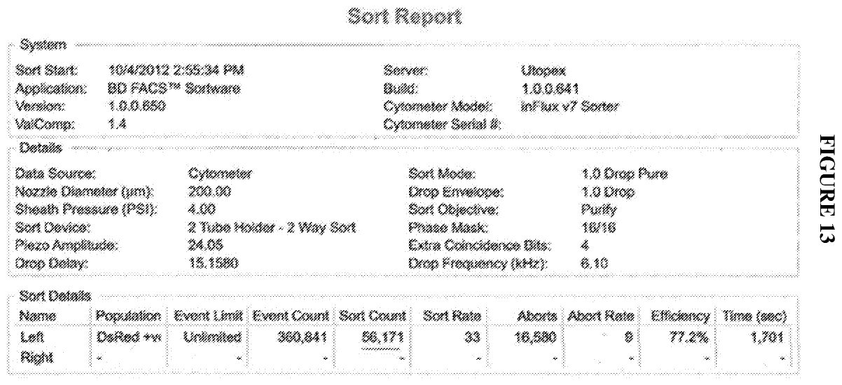

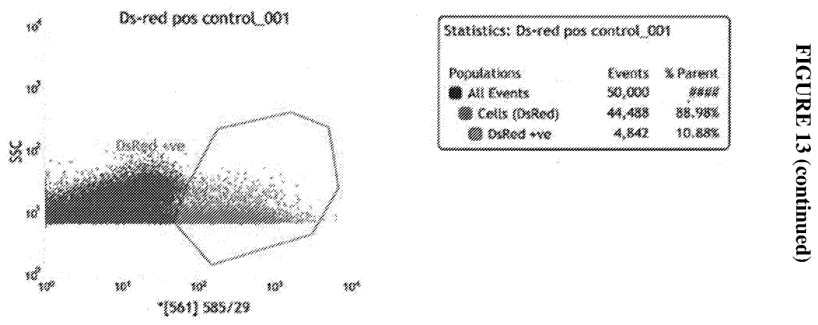

Isolated protoplasts were divided into aliquots of 1×10⁶ and centrifuged at 700 rpm for 10 min with the supernatant removed. The pelleted protoplasts are re-suspended by adding 100 μl of transformation buffer (Table 9) to the protoplast pellet, followed by 50 μl of 20-30 μg plasmid DNA and immediately 150 μl of pre-warmed 40% PEG solution (warmed to 42° C. for 1 hr prior to transformation; Table 10). Mixing gently after adding each of the contents and incubate at ambient room temperature (22° C.) for 15 minutes in the dark. Following the incubation, 5 ml of W5 buffer was added dropwise to the sides of the tube to gently mix the protoplasts. A further 5 ml of W5 was added as gently as possible to the sides of the tube to gently mix the protoplast. The protoplasts were centrifuged again at 700 RPM for 10 minutes; with the supernatant carefully discarded without disturbing the pellet and re-suspending in 150 μl of W5 buffer and incubated in the dark at room temperature (22° C.) for 48 hrs. The expression of the GFP and dsRED proteins was observed under a fluorescence microscope (OLYMPUS CKX53, Tokyo, Japan) (excitation emission wavelengths 470-490 and 550-570 nm) (FIG. 12). Transformation efficiency was calculated using a BD Influx FACS-based analysis with laser and filter sources (488 nm coherent sapphire sold-state laser, filter settings 517/518 nm for GFP) (FIG. 13).

The transfected protoplasts were collected into individual 1.5 or 2.0 ml microfuge tubes of c. 1.0×10⁶ protoplasts/ml per tube. The cells were then pelleted by centrifugation and a lysis buffer added, followed by snap freezing in liquid nitrogen. The cells were then subjected to DNA extraction following the Qiagen (Hilden Germany) DNeasy plant kit following manufacturer's instructions. The target genome edit sites were targeted by multiple pairs of PCR primers that generate amplicons that surround the site, which can be sequenced by 100-200 bp Illumina sequencing technology. Due to the requirement to cover all possible deletions the primers are required to be a minimum of 10-20 bp away from the target site. The amplicons comprising specific DNA bar codes were added to each tube and then pooled for DNA sequencing on an Illumina sequencing by synthesis platform. Sequence data of c. 10 million reads per sample were generated and subsequently aligned to the reference sequence, with variant sequences detected at the target site. A count of specific deletions at the target nuclease site was made in comparison to the number of unedited reference sequences. The size of the deletion could then be determined for each of the edited reads and the construct with the highest number of edits at the site identified for further stable editing.

### Media Composition

TABLE 7
Digestion media

| | | |
|---|---|---|
| Component | Concentration | pH |

| | | | |
|---|---|---|---|
| MES | 20 | mM | 5.8 |

| | |
|---|---|
| Mannitol | 0.5M |

| | | |
|---|---|---|
| KCl | 20 | mM |
| CaCl₂ | 10 | mM |
| Cellulase R-10 | 1-2.5% | (w/v) |
| Macerozyme R-10 | 0.2-0.5% | (w/v) |
| Pectolyase Y-23 | 0.2% | (w/v) |

To prepare the digestion media, the components (excluding enzymes) were mixed in MilliQ water, pH balance and filtered through 0.22 μm filter. The enzymes were then added to desired concentrations, and dissolved. The mixture was allowed to sit in a 55° C. water bath for 10 mins to enhance solubility, followed by 0.22 μm filtration.

TABLE 8
W5 wash buffer

| | | |
|---|---|---|
| Component | Concentration | pH |

| | | | |
|---|---|---|---|
| Glucose | 5 | mM | 5.8 |
| KCl | 5 | mM | |
| MES | 10 | mM | |
| CaCl₂ | 125 | mM | |
| NaCl₂ | 154 | mM | |

To prepare the W5 wash buffer, all components were mixed in MilliQ, pH balanced and filter sterilised through 0.22 μm filter.

TABLE 9
Transformation buffer

| | | |
|---|---|---|
| Component | Concentration | pH |

| | | | |
|---|---|---|---|
| MgCl₂ | 15 | mM | 5.8 |

| | |
|---|---|
| Mannitol | 0.5M |

| | | |
|---|---|---|
| MES | 0.1% | (w/v) |

The transformation buffer was prepared fresh for every transformation. 30 mL aliquots of the transformation buffer were prepared, pH balanced and filter sterilised through 0.22 μm filter syringe.

TABLE 10
PEG 4000

| Component | Concentration | pH |
|---|---|---|
| Ca(NO₃)₂4H₂O | 0.1M | — |
| Mannitol | 0.4M | |
| PEG 4000 | 40% (w/v) | |

The PEG 400 was prepared fresh for every transformation. 30 mL aliquots were prepared in MilliQ water and placed in a falcon tube. The falcon tube was then placed in water bath at 42° C. for 1 hour before transformation.

### Alternative Media Compositions

Cannabis strains have proven to be highly variable, making the effect of protocols, more importantly media compositions, variable across different strains. Here are potential alternative media compositions that can substitute the compositions previously mentioned, should those media not be suitable for the specific plants used (Table 11, 12 and 13).

TABLE 11
Digestion media

| | | |
|---|---|---|
| Component | Concentration | pH |

| | | | |
|---|---|---|---|
| MES | 20 | mM | 5.8 |

| | |
|---|---|
| Mannitol | 0.5M |

| | | |
|---|---|---|
| KCl | 20 | mM |
| CaCl₂ | 10 | mM |
| Cellulase R-10 | 1.5-2.5% | (w/v) |
| Macerozyme R-10 | 0.2-0.5% | (w/v) |
| Pectolyase | 0.2% | (w/v) |

### Protoplast Washing Buffers

TABLE 12
W1

| Component | Concentration | pH |
|---|---|---|
| KCl | 20 mM | 5.8 |
| MES | 4 mM | |
| Mannitol | 0.5M | |

TABLE 13
W11

| Component | Concentration | pH |
|---|---|---|
| KCl | 5 mM | 5.8 |
| MES | 2 mM | |
| CaCl₂ | 125 mM | |
| NaCl | 154 mM | |

### Example 3— Stable Transformation of Embryogenic Plant Tissues and Regeneration of Whole Plants Enabling Genome Editing

### Agrobacterium-mediated Transformation of Embryogenic Tissues and Callus

Seed germination and callus induction from embryogenic cotyledons

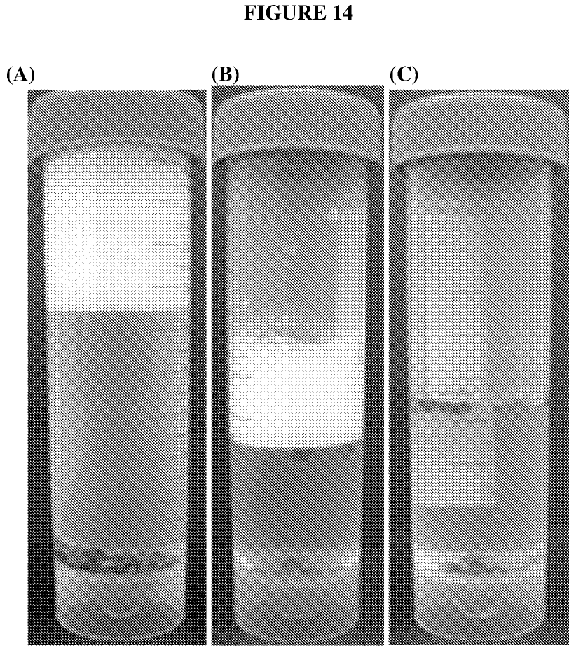

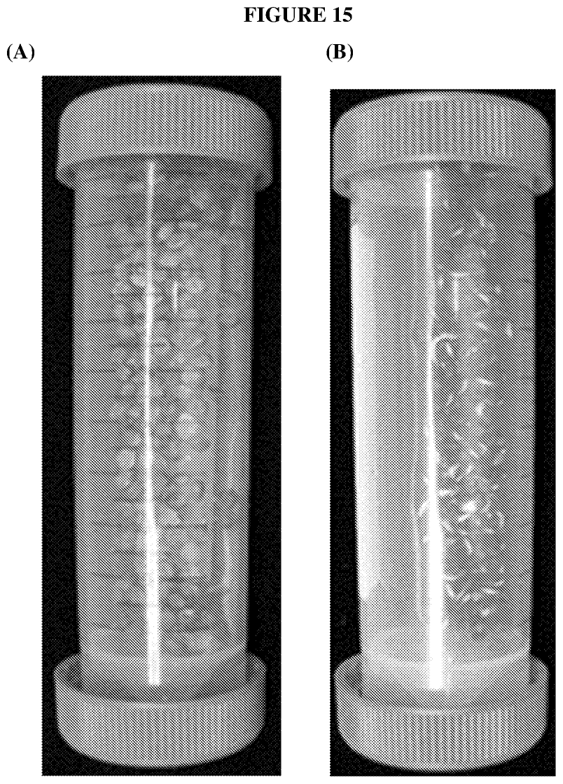

Seeds were initially cleaned by rinsing several times under tap water, then surface sterilised by stirring in 80% Ethanol (v/v) for 1 minute. The ethanol was then decanted off from the seeds, which were rinsed with tap water for a minimum of three times, changing the water in between each rinse. The seeds were then immersed in 15% Domestos® (4.75% available Chlorine m/v) for 15 min with shaking at 180 rpm/min (FIG. 14). The material at this point was transferred to an aseptic laminar flow cabinet, where the Domestos® was decanted away from the cannabis seeds that were rinsed repeatedly with sterile Milli-Q water, until the sterilising agent was completely removed, as indicated by a lack of white foam on the top of the water. The cannabis seeds were then transferred to a sterile 50 ml tube with 15 ml of sterile Milli-Q water and incubated at 25° C. for 3 to 7 days in the dark with the tubes placed horizontally (FIG. 15).

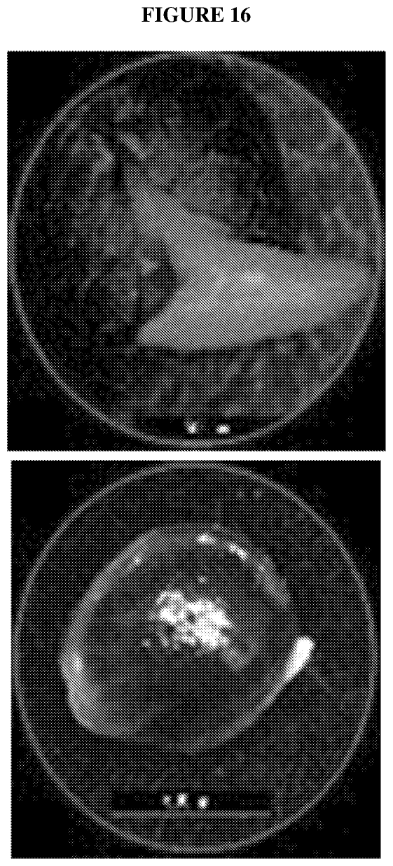

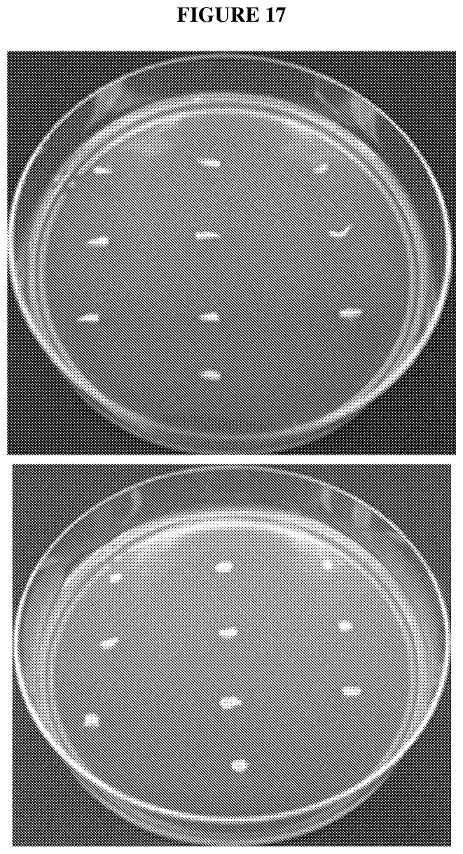

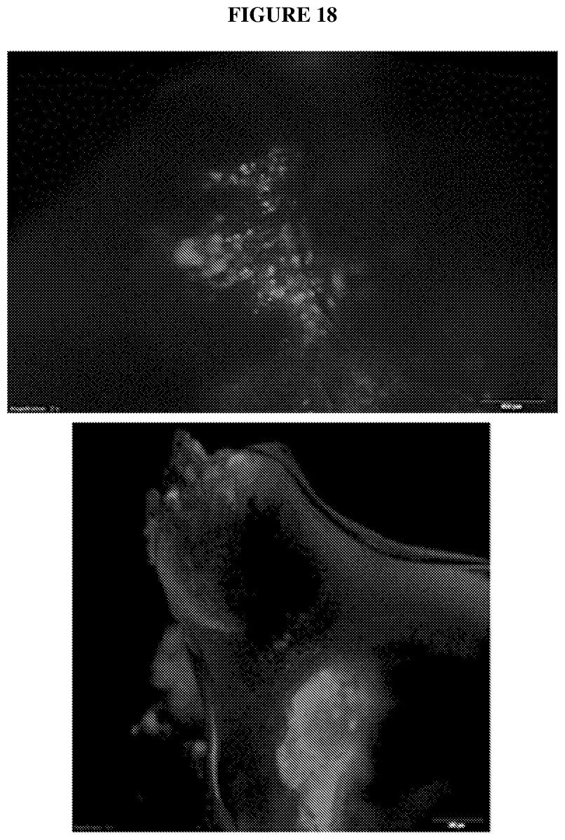

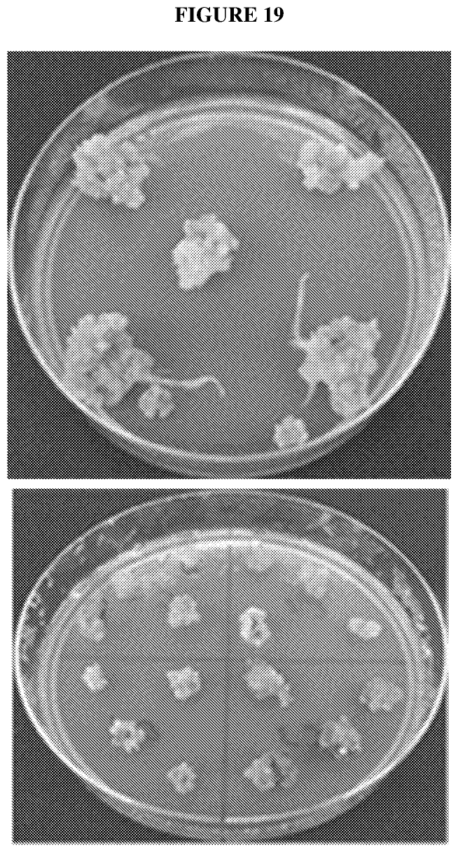

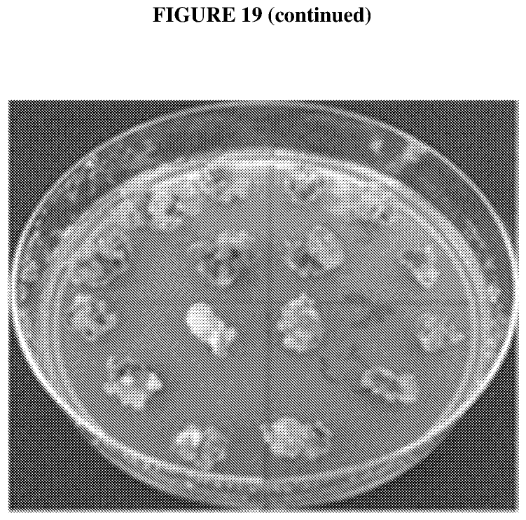

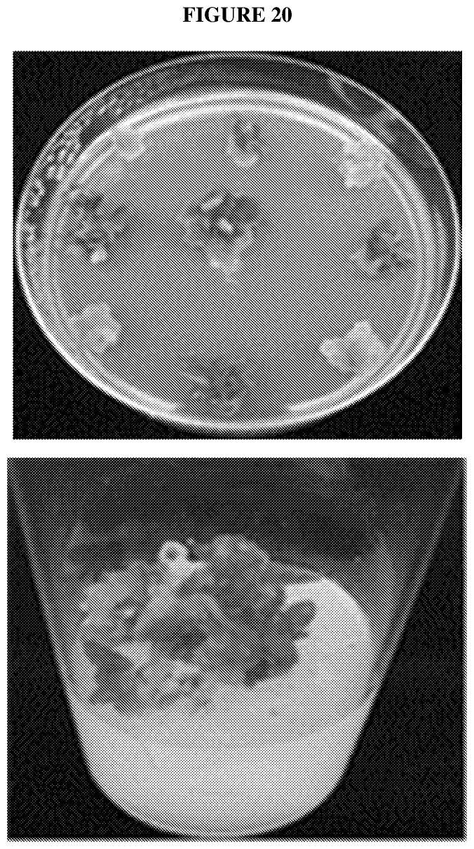

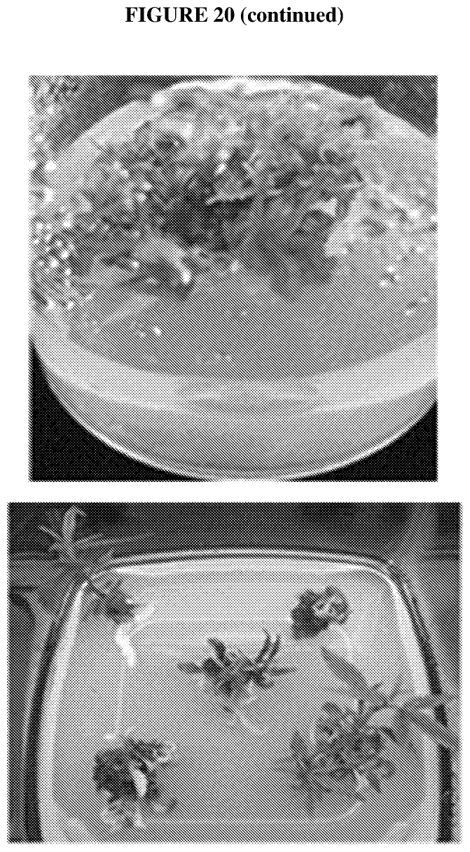

Once the seed coat has been split by the germinating seed (FIGS. 15 and 16) the cotyledons were excised from the seeds, under stereomicroscopy. The cannabis seeds were placed on a sterile Petri dish with the split seed coat facing upwards. A sterile scalpel blade was used to gently break and remove the cracked seed coat. The exposed tissue was then dissected into the embryogenic cotyledons and radicle of mature seeds before transfer to callus induction media (Table 14) with the addition of Timentin at 120 mg/L. The cut surface of the cotyledon was placed in an Agrobacterium tumefaciens liquid culture within a micro-centrifuge tube. Agrobacterium tumefaciens was cultured in LB medium with the addition of 50 μg/mL Spectinomycin and 25 μg/mL Rifampicin, at 28° C. with shaking at 180 rpm/min for 2 days prior to inoculation. The culture was normalised as an inoculation solution with the inclusion of 2 mg/L 2,4-D and adjusting the cell density by measuring and adjusting the optical density of the inoculation solution to an OD₆₀₀=0.5. Plates were incubated in the dark at 26°±1° C. for 2-4 weeks (FIG. 17), while stable transformation was observed 3-4 days post transformation (FIG. 18). Healthy callus (FIG. 19) was transferred onto regeneration media (Table 15) and maintained with 1 round of subculture after 2 weeks. Following callus induction and multiplication, further dissection to increase the transformed cells selected was performed. Callus was maintained on regeneration media for 4 weeks, to allow point root and shoot initiation to take place (FIG. 19). Callus with developing regenerating shoots (FIG. 20) were transferred onto rooting media (Table 16) in culture vessel for further root development (approx. 4-6 weeks).

Planting out and hardening of regenerated plantlets

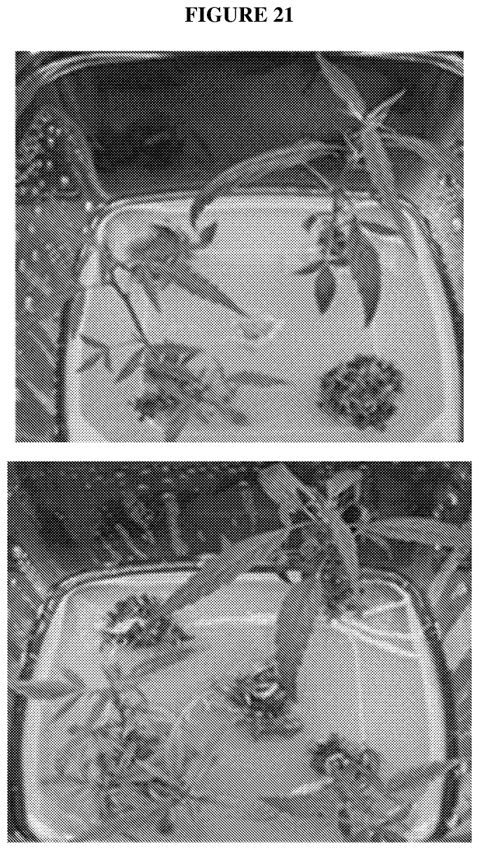

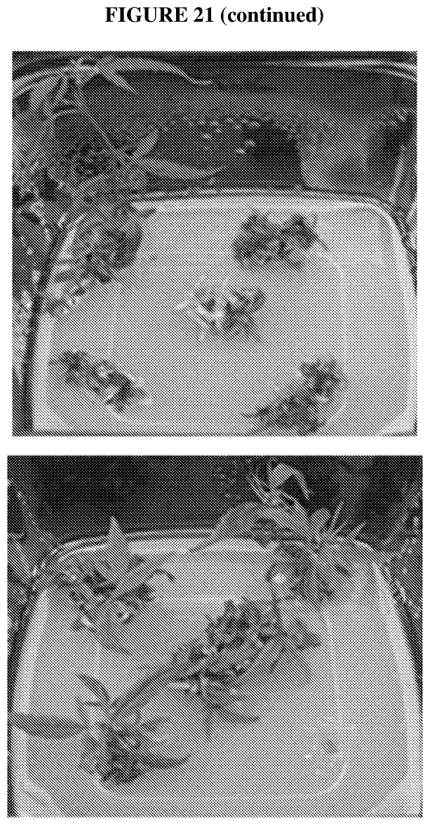

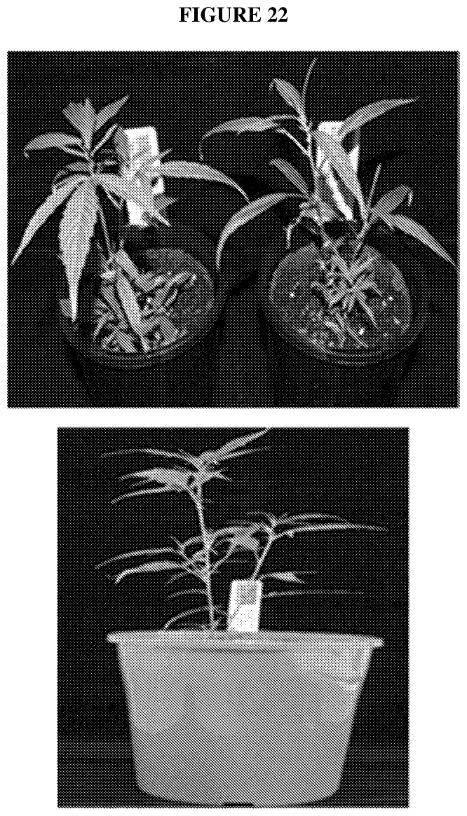

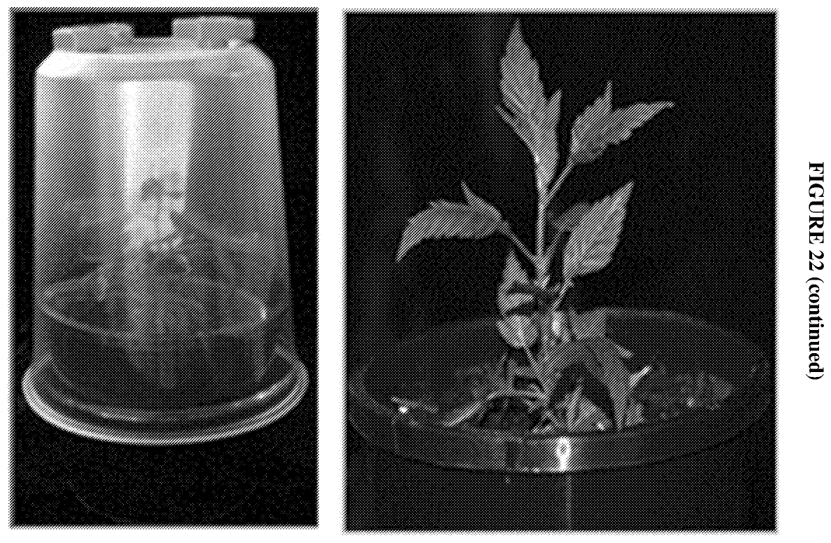

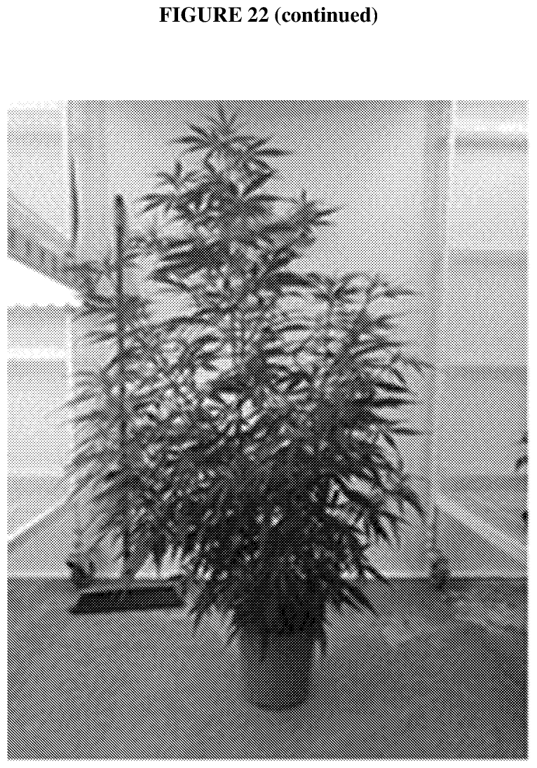

Healthy, rooted plantlets (FIG. 21) were transferred into small plastic potting containers containing potting mix and a humidity dome (FIG. 22) and watered sparingly for 1 week with the ventilation closed under an 18 hr light period at 26°±1° C. The next week, the ventilation was slowly opened and eventually removed allowing full acclimatisation to the environment. After 5-6 months, harvesting of seeds/flowers was achieved.

TABLE 14
Callus induction media

| | |
|---|---|
| Concentration | pH |

| | | | |
|---|---|---|---|
| Murashige and Skoog | 4.4 | g/L | 5.8 |
| Sucrose | 3% | (w/v) | |
| Agar | 0.7% | (w/v) | |
| Kinetin | 1 | mg/L | |
| NAA | 0.2 | mg/L | |

To prepare the callus induction media, all components were mixed together (excluding Kinetin and NAA) in MilliQ, pH balanced and autoclaved at 121° C. for 15 min. Once cooled to 55° C., the Kinetin and NAA were added, before pouring the media into sterile petri dishes.

TABLE 15
Regeneration media

| | | |
|---|---|---|
| Component | Concentration | pH |

| | | | |
|---|---|---|---|
| Murashige and Skoog | 4.4 | g/L | 5.8 |
| Maltose | 3% | (w/v) | |
| Agar | 0.7% | (w/v) | |
| TDZ | 1 | mg/L | |
| MES | 10 | mM | |
| My-inositol | 0.1 | g | |

To prepare the regeneration media, the components were mixed together (excluding TDZ) in MilliQ, pH balanced and autoclaved at 121° C. for 15 min Once cooled to 55° C., the TDZ was added, before pouring the media into sterile petri dishes/culture vessels.

TABLE 16
Rooting media

| | | |
|---|---|---|
| Component | Concentration | pH |

| | | | |
|---|---|---|---|
| Murashige and Skoog | 2.2 | g/L | 5.8 |
| (½ strength) | | | |
| Sucrose | 1% | (w/v) | |
| Agar | 1% | (w/v) | |
| IBA | 1 | mg/L | |

To prepare the rooting media, the components were mixed together (excluding IBA) in MilliQ, pH balanced and autoclaved at 121° C. for 15 min Once cooled to 55° C., IBA was added, before pouring the median into sterile culture vessels.

### Alternative Media Compositions

Again, each cannabis strain requires different hormones and carbohydrate sources to initiate undifferentiated callus formation and regeneration. All media for tissue culture requires a carbohydrate source (e.g., maltose replaces sucrose), agar concentration, and potentially agar source, hormone choice and concentration empirically adjusted on a plant genotype-by-genotype basis.

1. A method of producing a nucleic acid sequence encoding a DNA-recognition moiety for a targeted gene editing construct, the method comprising:
a. providing a nucleic acid sequence of a genome from a plant of a reference species;
b. providing a corresponding nucleic acid sequence of a genome from one or more additional plants of the reference species;
c. generating a consensus sequence of the nucleic acid sequences of (a) and (b);
d. identifying regions of genomic variation within the consensus sequence of (c); and
e. producing a nucleic acid sequence encoding a DNA-recognition moiety that is complementary to a target DNA sequence within the consensus sequence of (c), wherein the DNA-recognition moiety is not complementary to a region of genomic variation identified in (d).

2. The method of claim 1, the reference species is of the genus Cannabis.

3. The method of claim 2, wherein the reference species is Cannabis sativa.

4. The method of claim 3, wherein the genome of the reference species comprises one or more nucleic acid sequences selected from the group consisting of SEQ ID NOs: 164-198.

5. The method of claim 2, wherein the target DNA sequence comprises one or more cannabinoid biosynthesis genes.

6. The method of claim 5, wherein the one or more cannabinoid biosynthesis genes is selected from the group consisting of DXS1, DXS2, DXR, MCT, CMK, MDS, HDS, HDR, IPP/IPI, GPP_LSU, GPP_SSU, FAD2#1, FAD2#2, FAD2#3, FAD2#4, LOX, HPL, AAE], OLS, OAC, OAC#2, GOT, CBCAS, CBCAS-like#a, CBCAS-like#b, CBCAS-like#c, CBCAS-like#d, CBCAS-like#e, CBCAS-like#f, CBCAS-like#g, CBCAS#a, CBCAS#b, and THCAS.

7. The method of claim 1, wherein the genomic variation is selected from the group consisting of a single nucleotide polymorphism (SNP) location, SNP frequency, copy number and presence absence variations (PAV).

8. The method of claim 7, wherein the genomic variation is a genomic variation shown in any one of the sequences selected from the group consisting of SEQ ID NO: 199-233.

9. A nucleic acid sequence encoding a DNA-recognition moiety produced by the method of claim 1.

10. A gene editing construct comprising the nucleic acid sequence encoding the DNA-recognition moiety of claim 9.

11. The gene editing construct of claim 10, further comprising a nucleic acid sequence encoding an endonuclease.

12. The gene editing construct of claim 11, wherein the endonuclease is selected from the group consisting of a zinc finger nuclease and a transcription activator-like effector nuclease. (TALEN).

13. A method for modulating gene expression in a plant cell, the method comprising:
a. providing a plant cell;
b. transfecting the plant cell with the gene editing construct of claim 10; and
c. culturing the transfected plant cell of (b) for a time and under conditions suitable to drive the functional expression of the gene editing construct in the plant cell.

14. The method of claim 13, wherein the plant cell is a protoplast.

15. The method of claim 13, wherein the gene editing construct transiently modulates the expression of one of more target genes in the plant cell.

16. A transformed plant cell comprising the gene editing construct of claim 10.

17. A method for producing a regenerable plant cell with modified gene expression, the method comprising:
a. providing germinated plant tissue comprising regenerable cells;
b. transforming the regenerable cells with the gene editing construct of claim 10;
c. culturing the transformed regenerable cells of (b) for a time and under conditions suitable to express the gene editing construct;
d. culturing the transformed regenerable cells of (c) for a time and under conditions suitable for callus formation to occur; and
e. culturing the callus formed in step (d) for a time and under conditions suitable to produce a rooted plantlet, wherein the rooted plantlet is capable of growing into a plant with modified gene expression.

18. The method of claim 17, wherein the germinated plant tissue is selected from the group consisting of embryogenic cotyledons, primordial root and radicle of mature embryos.

19. A plant comprising the transformed plant cell of claim 16.

20. A regenerable plant cell produced by the method of claim 17.
